(19) United States
(12) Patent Application Publication
(10) Pub. No.: US 2024/0131080 A1
(43) Pub. Date: Apr. 25, 2024

(54) METHOD FOR REPROGRAMMING HUMAN CELLS

(71) Applicant: Yissum Research Development Company of the Hebrew University of Jerusalem Ltd., Jerusalem (IL)

(72) Inventors: Yossi BUGANIM, Kiryat Ono (IL); Moriyah Naama SHACHAM, Beit Shemesh (IL)

(73) Assignee: Yissum Research Development Company of the Hebrew University of Jerusalem Ltd., Jerusalem (IL)

(21) Appl. No.: 18/537,944

(22) Filed: Dec. 13, 2023

Related U.S. Application Data
(63) Continuation of application No. PCT/IL2022/050631, filed on Jun. 13, 2022.
(60) Provisional application No. 63/210,030, filed on Jun. 13, 2021.

(51) Int. Cl. A61K 35/545; A61K 8/64; A61Q 19/00; C12N 5/073; C12N 15/86

(52) U.S. Cl. CPC A61K 35/545; A61K 8/64; A61Q 19/00; C12N 5/0605; C12N 15/86; C12N 2501/603; C12N 2501/604; C12N 2501/606; C12N 2502/00; C12N 2513/00; C12N 2533/92; C12N 2740/15043

(57) ABSTRACT

A method of generating an induced trophoblast stem cell (iTSC) from a human cell is provided. Accordingly there is provided a method comprising expressing within a human cell GATA3 and OCT4 transcription factors, under conditions which allow generation of an iTSC from the cell. Also provided is a method of rejuvenating and/or de-differentiating a human cell. Also provided are nucleic acid constructs, protein preparation, isolated human cells, human iTSCs, rejuvenated cells and de-differentiated cells.

### RELATED APPLICATIONS

This application is a Continuation of PCT Patent Application No. PCT/IL2022/050631 having International filing date of Jun. 13, 2022 which claims the benefit of priority under 35 USC § 119(e) of U.S. Provisional Patent Application No. 63/210,030 filed on Jun. 13, 2021. The contents of the above applications are all incorporated by reference as if fully set forth herein in their entirety.

### SEQUENCE LISTING STATEMENT

The XML file, entitled 98707SequenceListing.xml, created on Dec. 7, 2023, comprising 152,445 bytes, submitted concurrently with the filing of this application is incorporated herein by reference.

### FIELD AND BACKGROUND OF THE INVENTION

The present invention, in some embodiments thereof, relates to a method for reprogramming human cells and, more particularly, but not exclusively, to a method for reprogramming human cells to induced trophoblast stem cells (iTSC) or to rejuvenate cells.

Regenerative medicine is a new and expanding discipline that aims at replacing lost or damaged cells, tissues or organs in the human body through cellular transplantation. Embryonic stem cells (ESCs) are pluripotent cells that are capable of long-term growth, self-renewal, and can give rise to every cell, tissue and organ in the fetus's body. Thus, ESCs hold great promise for cell therapy as a source of diverse differentiated cell types. Few major bottlenecks to realizing such potential are the risk of teratoma formation, allogenic immune rejection of ESC-derived cells by recipients and ethical issues. The discovery of induced pluripotent stem cells (iPSC) and the direct conversion approach opened an attractive avenue that resolves these problems.

Key master regulators are prevailing transcription factors that determine cell identity. Each cell type expresses a specific combination of key master regulators that together modulate the gene expression program of the cell. Alongside the master regulators, there are thousands of transcription factors, co-factors and chromatin modifiers which expression in the cell is crucial to maintain a stable cell state. The transcriptome of each cell type is tightly controlled by these factors to allow the cell to execute its function properly. The first report that demonstrated how powerful key master regulators are in modulating cell identity was in the 1980s, when Davis et al. showed that ectopic expression of MyoD in fibroblasts can convert them into myocyte-like cells [Davis ey al. Cell (1987) 51, 987-1000]. Almost twenty years later, Xie et al. demonstrated that forced expression of C/EBPα/β can convert differentiated B cells into macrophage-like cells [Xie et al. Cell (2004) 117, 663-676]. These two studies demonstrate how fragile and delicate the balance between cell identity and cell plasticity is, and suggest that, when overexpressed, key master regulators can alter cell fate.

In 2006, two Japanese scientists, Takahashi and Yamanaka, changed the way we used to think about cell plasticity when they showed that introduction of four transcription factors, Oct4, Sox2, Klf4 and Myc (OSKM), can reprogram fibroblasts into functional embryonic stem cell-like cells [also termed induced pluripotent stem cells (iPSCs)] [Takahashi, K., and Yamanaka, S. Cell (2006) 126, 663-676]. The notion that as little as four factors are sufficient to reset the epigenome of a cell, opened a new avenue where scientists have attempted to convert different adult cells into other somatic cell types from ontogenetically different lineages, by avoiding the pluripotent state, using a specific subset of key master regulators. Using this approach several subsets of cell types such as hematopoietic cells, different neuronal cells, cardiomyocytes, hepatocytes, embryonic Sertoli cells, endothelial cells and RPE were converted from different somatic cells.

In mammals, specialized cell types of the placenta mediate the physiological exchange between the fetus and mother during pregnancy. The precursors of these differentiated cells are trophoblast stem cells (TSCs). In the pre-implantation embryo, trophoblast cells are the first differentiated cells that can be distinguished from the pluripotent inner cell mass, and form the outermost layer of the blastocyst [Roberts, R. M., and Fisher, S. J. Biology of reproduction (2011) 84, 412-421]. The trophoblast cell lineage is the source for the most essential cell types of the main structural and functional components of the placenta. Therefore, TSCs have tremendous biomedical relevance, as one third of all human pregnancies are affected by placental-related disorders [James et al. Placenta (2014) 35, 77-84].

In the mouse, TSCs can be isolated and cultured from outgrowths of either the blastocyst polar trophectoderm (TE) or extraembryonic ectoderm (ExE), which originates from the polar TE after implantation [e.g. Latos and Hemberger, (2014) Placenta. 35 Suppl: S81-5]. For an extensive period of time, all attempts to isolate and propagate human TSCs (hTSCs) in-vitro had failed. Very recently, hTSCs were successfully cultured for the first time [Okae et al., Cell stem cell (2018) 22, 50-63 e56]. These hTSCs gave rise to all major trophoblastic cell types following differentiation, exhibited transcriptional and epigenetic signatures similar to primary placental cells, and formed trophoblastic lesions when injected into NOD/SCID mice, suggesting fully functional hTSCs (Okae et al., 2018).

Generation of induced TSC-like cells (iTSCs) from embryonic stem cells (ESCs) and somatic cells e.g. fibroblast has been described before (Cambuli et al., 2014; Kuckenberg et al., 2010; Lu et al., 2008; Ng et al., 2008; Nishioka et al., 2009; Niwa et al., 2000; Niwa et al., 2005; Ralston et al., 2010; and 15-17); however, in all models lineage conversion remained incomplete and failed to confer a stable true TSC phenotype. Recently, transient ectopic expression of four mouse key trophectoderm (TE) genes, GATA3, Eomes, Tfap2c and Myc (GETM), were shown to reprogram fibroblasts to stable and fully functional mouse induced trophoblast stem cells [miTSCs, Benchetrit et al., Cell stem cell (2015)17, 543-556].

Additional background art includes:
U.S. Pat. No. 7,642,091;
U.S. Pat. No. 6,630,349;
US Application Publication No: US 20050191742;
International Application Publication No: WO 2006052646;
Canadian Patent Application Publication No: CA 2588088;
International Application Publication No WO2016/005985; and
Fogarty et al. Nature (2017) 550(7674): 67-73.

### SUMMARY OF THE INVENTION

According to an aspect of some embodiments of the present invention there is provided a method of generating an induced trophoblast stem cell (iTSC) from a human cell, the method comprising expressing within the cell exogenous GATA3 and OCT4 transcription factors, under conditions which allow generation of an iTSC from the cell, thereby generating the iTSC from the cell.

According to an aspect of some embodiments of the present invention there is provided a method of generating an induced trophoblast stem cell (iTSC) from a human cell, the method comprising expressing within the cell exogenous GATA3, OCT4 and KLF transcription factors, under conditions which allow generation of an iTSC from the cell, thereby generating the iTSC from the cell.

According to an aspect of some embodiments of the present invention there is provided a method of rejuvenating and/or de-differentiating a human cell, the method comprising expressing within the cell exogenous GATA3 and OCT4 transcription factors, under conditions which allow rejuvenation and/or de-differentiation of the cell, thereby generating a rejuvenated cell and/or a de-differentiated cell.

According to an aspect of some embodiments of the present invention there is provided a method of rejuvenating and/or de-differentiating a human cell, the method comprising expressing within the cell exogenous GATA3, OCT4 and KLF transcription factors, under conditions which allow rejuvenation and/or de-differentiation of the cell, thereby generating a rejuvenated cell and/or a de-differentiated cell.

According to some embodiments of the invention, the expressing comprises transiently expressing.

According to some embodiments of the invention, the method further comprising expressing within the cell an exogenous c-MYC transcription factor.

According to some embodiments of the invention, the method further comprising expressing within the cell an exogenous KLF4 transcription factor.

According to some embodiments of the invention, the method further comprising expressing within the cell an exogenous KLF transcription factors

According to some embodiments of the invention, the conditions are such that expressing is for at least 14 days following introducing the exogenous transcription factor into the cell.

According to some embodiments of the invention, the conditions are such that expressing is for no more than 30 days following introducing the exogenous transcription factor into the cell.

According to some embodiments of the invention, the conditions are such that expressing is for at least 1 day following introducing the exogenous transcription factor into the cell.

According to some embodiments of the invention, the conditions are such that expressing is for less than 25 days following introducing the exogenous transcription factor into the cell.

According to some embodiments of the invention, the iTSC does not express the exogenous transcription factor as determined by at least one of PCR, western blot and/or flow cytometry.

According to some embodiments of the invention, the rejuvenated cell and/or de-differentiated cell does not express the exogenous transcription factor as determined by at least one of PCR, western blot and/or flow cytometry.

According to some embodiments of the invention, the expressing comprises introducing into the cell a polynucleotide encoding the transcription factor.

According to some embodiments of the invention, the polynucleotide is a DNA.

According to some embodiments of the invention, the polynucleotide is a RNA.

According to some embodiments of the invention, the method comprising isolating the iTSC from non-iTSC.

According to some embodiments of the invention, the method comprising assaying generation of iTSC.

According to some embodiments of the invention, the method comprising isolating the rejuvenated cell from non-rejuvenated cell.

According to some embodiments of the invention, the method comprising assaying rejuvenation.

According to some embodiments of the invention, the method comprising isolating the de-differentiated cell from non-de-differentiated cell.

According to some embodiments of the invention, the method comprising assaying de-differentiation.

According to an aspect of some embodiments of the present invention there is provided a nucleic acid construct or system comprising at least one polynucleotide comprising a nucleic acid sequence encoding GATA3 and OCT4 transcription factors.

According to an aspect of some embodiments of the present invention there is provided a nucleic acid construct or system comprising at least one polynucleotide comprising a nucleic acid sequence encoding GATA3, OCT4 and KLF transcription factors.

According to some embodiments of the invention, the at least one polynucleotide further comprises a nucleic acid sequence encoding a c-MYC transcription factor.

According to some embodiments of the invention, the at least one polynucleotide further comprises a nucleic acid sequence encoding a KLF4 transcription factor.

According to some embodiments of the invention, the at least one polynucleotide further comprises a nucleic acid sequence encoding a KLF transcription factor.

According to some embodiments of the invention, the at least one polynucleotide is a RNA.

According to an aspect of some embodiments of the present invention there is provided a protein preparation comprising GATA3 and OCT4 transcription factors polypeptides to a level of purity of at least 20%.

According to an aspect of some embodiments of the present invention there is provided a protein preparation comprising GATA3, OCT4 and KLF transcription factors polypeptides to a level of purity of at least 20%.

According to some embodiments of the invention, the protein preparation further comprising a c-MYC transcription factor polypeptide.

According to some embodiments of the invention, the protein preparation further comprising KLF4 transcription factor polypeptide.

According to some embodiments of the invention, the protein preparation further comprising a KLF transcription factor polypeptide.

According to an aspect of some embodiments of the present invention there is provided an isolated human cell expressing exogenous GATA3 and OCT4 transcription factors.

According to an aspect of some embodiments of the present invention there is provided an isolated human cell expressing exogenous GATA3, OCT4 and KLF transcription factors.

According to some embodiments of the invention, the isolated cell further expressing an exogenous c-MYC transcription factor.

According to some embodiments of the invention, the isolated cell further expressing an exogenous KLF4 transcription factor.

According to some embodiments of the invention, the isolated cell further expressing an exogenous KLF transcription factor.

According to some embodiments of the invention, the cell comprises a DNA molecule encoding the transcription factor.

According to some embodiments of the invention, the cell comprises a RNA molecule encoding the transcription factor.

According to some embodiments of the invention, the RNA is a modified RNA.

According to some embodiments of the invention, the cell comprises a protein molecule of the transcription factor.

According to some embodiments of the invention, the expressing is not in the natural location and/or expression level of the native gene of the transcription factor.

According to some embodiments of the invention, the cell is a somatic cell.

According to some embodiments of the invention, the cell is a fibroblast.

According to some embodiments of the invention, the cell is selected from the group consisting of keratinocyte, hematopoietic cell, retinal cell, fibroblast, hepatocyte, cardiac cell, kidney cell, pancreatic cell and neuron.

According to some embodiments of the invention, the cell is hematopoietic cell or mesenchymal stem cell.

According to an aspect of some embodiments of the present invention there is provided an isolated induced trophoblast stem cell (iTSC) obtainable according to the method.

According to an aspect of some embodiments of the present invention there is provided an isolated rejuvenated and/or de-differentiated cell obtainable according to the method.

According to an aspect of some embodiments of the present invention there is provided an isolated human induced trophoblast stem cell (iTSC) comprising an ectopic DNA of GATA3 and OCT4 transcription factors integrated in the genome.

According to an aspect of some embodiments of the present invention there is provided an isolated human induced trophoblast stem cell (iTSC) comprising an ectopic DNA of GATA3, OCT4 and KLF transcription factors integrated in the genome.

According to some embodiments of the invention, the cell further comprises an ectopic DNA of a c-MYC transcription factor integrated in the genome.

According to some embodiments of the invention, the cell further comprises an ectopic DNA of a KLF4 transcription factor integrated in the genome.

According to some embodiments of the invention, the cell further comprises an ectopic DNA of a KLF transcription factor integrated in the genome.

According to some embodiments of the invention, the isolated iTSC maintaining differentiation level of a trophoblast stem cell for at least 20 passages in culture.

According to some embodiments of the invention, the iTSC is characterized by at least one of:
(i) TSC morphology;
(ii) TSC markers, as determined by an immunocytochemistry and/or PCR assay;
(iii) absence of fibroblast specific markers, as determined by an immunocytochemistry and/or PCR assay;
(iv) a transcriptome similar to a blastocyst-derived TSC, as determined by a RNA sequencing assay;
(v) genomic stability similar to a blastocyst-derived TSC, as determined by Chromosomal Microarray Analysis;

(vi) a methylation pattern similar to a blastocyst-derived TSC, as determined by a bisulfate assay;

(vii) in-vitro differentiation following culture in a medium without factors supporting the undifferentiated state or in a medium conducive to directed differentiation, as determined by morphology, flow cytometry and/or PCR assay;

(viii) in-vitro and/or in-vivo differentiation into derivatives of the trophectoderm lineage, as determined by morphology, immunocytochemistry, immunocytochemistry, flow cytometry and/or PCR assay;

(ix) Ability to form a three dimensional organoid culture, as determined by morphology, immunocytochemistry, immunocytochemistry and/or PCR assay;

(x) in-vivo formation of a trophoblastic lesion, as determined by histological evaluation;

(xi) no change in differentiation level for at least 20 passages in culture as determined by at least one of the assay in (i)-(x).

According to some embodiments of the invention, the methylation pattern comprises hypomethylation of the ELF5 promoter region, and/or hypermethylation of the Nanog promoter as compared to a somatic cell and/or an ESC cell.

According to some embodiments of the invention, the rejuvenated cell is characterized by at least one of:
(i) a morphology of a cell of the same type and developmental stage that has not been subjected to the method;
(ii) markers of a cell of the same type and developmental stage that has not been subjected to the method, as determined by an immunostaining, western blot and/or PCR assay;
(iii) a transcriptome, with the exception of genes that are associated with age, similar to a cell of the same type and developmental stage that has not been subjected to the method, with the exception of genes that as determined by a RNA sequencing assay;
(iv) a methylation pattern distinct from a cell of the same type and developmental stage that has not been subjected to the method, as determined by a bisulfate assay.

According to an aspect of some embodiments of the present invention there is provided a cell culture comprising the isolated cell and a culture medium.

According to an aspect of some embodiments of the present invention there is provided a cell culture comprising the isolated cell and a culture medium.

According to some embodiments of the invention, the culture medium comprises a composition of components that have been shown to support culture of human TSCs.

According to some embodiments of the invention, the isolated cell being a cell line.

According to an aspect of some embodiments of the present invention there is provided a cell line of the isolated cell.

According to an aspect of some embodiments of the present invention there is provided an isolated population of cells, wherein at least 80% of the cells are the iTSCs.

According to an aspect of some embodiments of the present invention there is provided an isolated population of cells, wherein at least 80% of the cells are the rejuvenated and/or de-differentiated cells.

According to an aspect of some embodiments of the present invention there is provided an isolated population of cells, wherein at least 80% of the cells are the cells disclosed herein.

According to an aspect of some embodiments of the present invention there is provided a pharmaceutical composition comprising the iTSC or the population of cells and a pharmaceutically acceptable carrier or diluent.

According to an aspect of some embodiments of the present invention there is provided a pharmaceutical composition comprising the construct or system or the protein preparation and a pharmaceutically acceptable carrier or diluent.

According to an aspect of some embodiments of the present invention there is provided a cosmetic composition comprising the construct or system or the protein preparation and a cosmetic carrier or diluent.

According to some embodiments of the invention, the cosmetic being formulated as a cream, a face mask, a scrub, a soap, a wash or a gel.

According to an aspect of some embodiments of the present invention there is provided an isolated aggregate, organoid, placenta, developing embryo or synthetic embryo comprising the iTSC, the construct or system or the protein preparation.

According to an aspect of some embodiments of the present invention there is provided a method of augmenting a placenta, a developing embryo or a synthetic embryo comprising introducing into a placenta, a developing embryo or a synthetic embryo the iTSC, the construct or system or the protein preparation.

According to an aspect of some embodiments of the present invention there is provided a method of generating an aggregate or organoid comprising trophoblasts, the method comprising introducing into a scaffold or a matrix the iTSC, the construct or system or the protein preparation.

According to an aspect of some embodiments of the present invention there is provided a method of treating and/or preventing a disorder associated with development and/or activity of trophoblasts in a subject in need thereof, the method comprising administering to the subject a therapeutically effective amount of the iTSC or the population of cells, the pharmaceutical composition, the construct or system or the protein preparation, thereby treating and/or preventing the disorder associated with development and/or activity of trophoblasts in the subject.

According to an aspect of some embodiments of the present invention there is provided a method of treating and/or preventing a disease associated with aging in a subject in need thereof, the method comprising administering to the subject a therapeutically effective amount of the cell or the population of cells, the pharmaceutical composition, the construct or system or the protein preparation, thereby treating and/or preventing the disease in the subject.

According to some embodiments of the invention, the disease is a vision-related disease.

According to some embodiments of the invention, the disease is selected from the group consisting of glaucoma, cataract, high myopia, retinitis pigmentosa, cone dystrophy, cone-rod dystrophy, Usher syndrome, Stargardt disease, Barder-Biedell syndrome, Best disease and inherited maculopathy.

According to some embodiments of the invention, the disease is selected from the group consisting of Myelodysplastic syndromes (MDS), cancer, graft rejection, graft versus host disease (GVHD), infectious disease, cytokine storm, radiation damage, neurodegenerative disease and wound.

According to an aspect of some embodiments of the present invention there is provided a method of performing a cosmetic care in a subject in need thereof, the method comprising applying to the skin of the subject a therapeutically effective amount of the construct or system, the protein preparation or the cosmetic composition, thereby performing the cosmetic care.

According to some embodiments of the invention, the KLF transcription factor is selected from the group consisting of KLF4, KLF5, KLF6 and KLF15.

According to some embodiments of the invention, the KLF transcription factors is selected from the group consisting of KLF4 and KLF5.

According to some embodiments of the invention, the KLF transcription factor comprises at least two distinct KLF transcription factors.

According to some embodiments of the invention, the KLF transcription factor comprises at least KLF4 and KLF5.

According to an aspect of some embodiments of the present invention there is provided a method of identifying an agent capable of modulating trophoblast development and/or activity, the method comprising:
(i) contacting the isolated iTSC or the population of cells, the aggregate, organoid or placenta with a candidate agent; and
(ii) comparing development and/or activity of the isolated iTSC, population of cell, aggregate, organoid or placenta following the contacting with the agent to development and/or activity of the isolated iTSC, population of cells, aggregate, organoid or placenta without the agent,
wherein an effect of the agent on the development and/or activity of the isolated iTSC, population of cell, aggregate, organoid or placenta above a predetermined level relative to the development and/or activity of the isolated iTSC, population of cells, aggregate, organoid or placenta without the agent is indicative that the drug modulates trophoblast development and/or activity.

According to an aspect of some embodiments of the present invention there is provided a method of obtaining a compound produced by a trophoblast, the method comprising culturing the isolated iTSC, the population of cells or the cell culture and isolating from the culture medium a compound secreted by the cells, thereby obtaining the compound produced by the trophoblast.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

### BRIEF DESCRIPTION OF THE SEVERAL VIEWS OF THE DRAWINGS

The patent or application file contains at least one drawing executed in color. Copies of this patent or patent application publication with color drawing(s) will be provided by the Office upon request and payment of the necessary fee.

Some embodiments of the invention are herein described, by way of example only, with reference to the accompanying drawings. With specific reference now to the drawings in detail, it is stressed that the particulars shown are by way of example and for purposes of illustrative discussion of embodiments of the invention. In this regard, the description taken with the drawings makes apparent to those skilled in the art how embodiments of the invention may be practiced.

FIGS. 1A-E demonstrate that ectopic expression of GATA3, OCT4, KLF4 and MYC (GOKM) convert human fibroblasts into trophoblast stem-like cells. (A) Schematic representation of the protocol for reprogramming human foreskin fibroblasts (HFFs, KEN or PCS201) into human induced trophoblast stem cells (hiTSCs). M2rtTA-containing HFFs, passage 7-14, were infected with lentiviral vectors encoding for the indicated transcription factors. Infected HFFs were exposed to doxycycline (dox) for 28 days, while the relevant medium was changed as depicted in the scheme. 7-10 days post dox withdrawal, stable epithelial colonies were collected and seeded on feeder-containing plate. Colonies were passaged until full stabilization. (B) Bright field images of two human blastocyst-derived TSC lines, hbdTSC#2 and hbdTSC#9, and four representative hiTSC colonies originating either from KEN, hiTSC#1 and hiTSC34, or from PSC201, hiTSC#11 and hiTSC#12, HFF lines. (C-D) qPCR analysis of mRNA levels for TSC-specific, TFAP2C, TP63, KRT7 and endogenous GATA3 (C) and mesenchymal-specific genes, THY 1, ZEB1, VIM and ACTA2, in four hiTSC colonies, two hbdTSC lines, 2 HFF lines, hESCs and iPSCs. Results are shown relative to the highest expressing sample and normalized to the mRNA levels of the housekeeping control gene GAPDH. Bars indicate standard deviation between technical duplicates. A typical experiment out of 3 independent experiments is shown. (E) Immunofluorescent staining of PFA-fixated hbdTSC line, hbdTSC#9, and representative hiTSC clone, hiTSC#1, for the TSC markers GATA2, GATA3, KRT7, the epithelial markers KRT18 and CDH1, and the mesenchymal marker VIM. Experiment was repeated with two hbdTSC lines and two hiTSC lines with similar results.

FIGS. 2A-C show RNAseq analysis results indicating that hiTSCs and hbdTSCs have highly similar transcriptomes. (A-C) Plots portraying comparisons of whole transcriptome, based on RNA-seq data, of two biological duplicates of HFFs, hESCs, hiPSCs, two hbdTSC lines, hbdTSC#2 and hbdTSC#9, and three hiTSC clones, hiTSC#1, hiTSC#4 and hiTSC#7. Principal component analysis (PCA) plot (A), correlation heatmap (B) and scatter plots (C) of bulk RNA showing the transcriptional similarity between hbdTSCs and hiTSCs and their distance from pluripotent stem cells (PSCs) and HFFs. The hierarchical clustering in (B) was generated using the Spearman correlation coefficient of log2-CPM expression data values. Note that hiTSC lines cluster closer to hbdTSCs than hbdTSC lines do with each other. Pairwise scatterplot comparison (C) of the global gene expression profiles of hbdTSC#9 versus hESCs, HFFs, hbdTSC#2 and three hiTSC colonies, showing high correlation only between different colonies of hbdTSC and between different colonies of hbdTSC and hiTSC. Representative genes expressed in ESCs (NANOG, OCT4), fibroblasts (VIM, ZEB1), and hTSCs (GATA3, TP63, TEAD4, TFAP2C) are indicated.

FIGS. 3A-D show RRBS analysis demonstrating trophoblast-specific changes in methylation in hiTSCs. Methylation analysis of three biological replicates of HFFs, hESCs, two hbdTSC lines, hbdTSC#2 and hbdTSC#9, and four hiTSC clones, hiTSC#1, hiTSC#2, hiTSC#4 and hiTSC#11, as assessed by RRBS. Analysis of CpG methylation ratio with sequencing depth of at least 10 reads per tile was computed, based on 100 bp tiles. (A, Left) Heatmap showing 4676 differentially methylated regions (DMRs) of 100 bp hypomethylated in HFFs and hypermethylated in hbdTSCs with methylation difference above 50%. hiTSCs are shown to have successfully acquired virtually all methylation patterns similar to hbdTSC. (A, right) Boxplot showing the average methylation for each biological sample. (B, left) Heatmap showing 24205 DMRs of 100 bp hypermethylated in HFFs and hypomethylated in hbdTSCs with methylation difference above 50%. hiTSCs are shown to have successfully acquired the majority of methylation patterns similar to hbdTSC. (B, right) Boxplot showing the average methylation for each biological sample. (C) Genome browser capture of the methylation levels of various tiles as assessed by RRBS in the ELF5 locus. Note trophoblast-specific hypomethylation upstream of the ELF5 gene. (D) Genome browser capture of the methylation levels of various tiles as assessed by RRBS in the NANOG locus. Note PSC-specific hypomethylation upstream of the NANOG gene compared with hbdTSC and hiTSC samples. Black square indicates 1000 bps tile.

FIGS. 4A-E demonstrates that hiTSCs differentiate into multinucleated ST cells. (A) Flow cytometry analysis of propidium iodide (PI) nuclear-stained cells at day 0, 4 and 8 after switching to basic differentiation medium (BDM), consisting of DMEM supplemented with 10% FBS, indicating spontaneous differentiation and formation of multinucleated syncytia. Experiment was repeated with two hbdTSC lines and two hiTSC lines with similar results. (B) Bright field images of hbdTSC#2 and hiTSC#4 after 6 days in medium for directed differentiation of TSCs into syncytiotrophoblast (STM) (Okae et al., 2018). (C) qPCR analysis of relative mRNA levels of ST-specific markers, CSH1, GCM1, SDC1 and CGB for the indicated samples at days 0, 2 and 6 in STM. Results presented as fold change relative to highest expressed day of each colony and normalized to the housekeeping control gene GAPDH. (D and E) Immunofluorescent staining of PFA-fixated undifferentiated hbdTSC#2 and hiTSC#4 and their ST derivatives after 6 days of ST differentiation. Cells were stained for DAPI (blue), epithelial-specific protein CDH1 (green) and pan-trophoblast marker KRT7 (red, D) and CSH1 and SDC1 (green) as ST-specific markers (E). White arrows indicate spontaneous ST differentiation regions in the undifferentiated TSCs and yellow arrows indicate undifferentiated cells that are CDH1-positive in the ST differentiation plate.

FIGS. 5A-C demonstrate that hiTSCs differentiate into HLA-G-positive EVT cells. (A) Bright field images of hbdTSC#2, hiTSC#4 and hiTSC#2 and their EVT derivatives following 6 days of directed differentiation. (B) qPCR analysis of relative mRNA levels of EVT-specific markers, HLA-G, MMP2, ITGA5 and ITGA1 at days 0, 6 and 14 of directed differentiation into extravillous trophoblast. Results presented as fold change relative to highest expressed day of each colony and normalized to housekeeping control gene GAPDH. (C) Immunofluorescent staining of PFA-fixated undifferentiated hbdTSC#2 and hiTSC#4 and their EVT derivatives after 14 days of EVT differentiation. Cells were stained for DAPI (blue), epithelial-specific protein EPCAM (green) and EVT-specific marker HLA-G (red).

FIGS. 6A-C demonstrate that hiTSCs engrafted into NOD-SCID mice form trophoblastic lesions, hiTSCs can be used to establish three-dimensional organoid cultures. (A, left) Lesions extracted from NOD-SCID mice after subcutaneous injection of 4×10⁶ cells of hbdTSC#2 or hiTSC#3 lines. Lesions were collected nine days after injection. (A, right) Stained sections of trophoblastic lesions extracted from NOD-SCID mice. Hematoxylin and eosin staining and KRT7 immunohistochemical staining with hematoxylin counter staining is shown. (B, Left) Commercial pregnancy tests, which detect presence of hCG, displaying positive results in the medium of all hTSCs but negative in HFFs and PSCs. (B, Right) qPCR analysis of mRNA levels of CGB gene, which encodes for the beta subunit of the trophoblast-specific hormone hCG. Results presented as fold change relative to highest expressed sample and normalized to housekeeping control gene GAPDH. (C, Left) bright field images of hbdTSC#2 and hiTSC#4 at day 1 and 10 of organoid formation protocol. (C, Right) spinning disk confocal imaging of formed organoids following immunofluorescent staining of DAPI, pan-trophoblast marker KRT7 and the proliferative cell marker Ki-67. White arrows indicate area of differentiation that are KRT7-positive but KI-67-negative.

FIGS. 7A-G demonstrate that hiTSCs reprogrammed with forced expression of GOKM undergo MET and express trophoblast markers. (A) qPCR analysis of the indicated transgenes in the depicted hiTSC colonies and control, HFFs (KEN), HFFs (PCS) and hbdTSC#2. Transgene integration was assessed by designing forward primers for the last exon of the transgene with reverse primers matching the sequence of the FUW-tetO plasmid (See Table 1). Results are shown relative to highest sample and normalized to intronic regions of GAPDH gene. Bars indicate standard deviation between two duplicates. (B) qPCR analysis of mRNA levels of the indicated transgenes in infected HFFs, three days after dox exposure. Three independent infections are shown compared to uninfected HFFs. (C) qPCR analysis of mRNA levels of trophoblast markers GATA2 and TFAP2A. Note that TFAP2A and to some extent GATA2 are expressed in fibroblasts. (D) qPCR analysis of mRNA levels of HLA class I gene HLA-A normalized to the housekeeping control gene GAPDH. The results are presented as fold change when the highest sample was set to 1. (E) qPCR analysis of mRNA levels of epithelial markers KRT18, CDH1, OCLN and EPCAM in the indicated hiTSC colonies and controls, HFFs (KEN), HFFs (PCS), ESCs and iPSC#1. Results are shown relative to highest expressing sample for each gene and normalized to mRNA of GAPDH gene. Bars indicate standard deviation between technical duplicates in a typical experiment. (F, top) Immunofluorescent staining for DAPI and the TSC-specific marker TFAP2C in PFA-fixated hbdTSC#9, hiTSC#1 and HFF control. (F, bottom) immunofluorescent staining for DAPI, GATA3, KRT7, KRT18, CDH1 and VIM in PFA-fixated HFFs. (G) Histogram showing flow cytometry analysis of classical HLA class I protein expression (HLA-A/B/C) in the indicated hiTSC colonies, hbdTSC#2 and HFFs using the well-characterized W6/32 antibody. HFFs and hbdTSC#2 were stained with secondary antibody only for control for non-specific staining.

FIGS. 8A-C show RNA-seq analysis indicating that hiTSCs and hbdTSCs have transcriptomes enriched for gene ontology term related to placental development. (A and B) Differentially expressed genes between hTSCs (hbdTSCs or hiTSCs) and hESCs and HFFs revealed significant enrichment for gene ontology terms relevant to placenta and embryonic placenta morphogenesis and development according to Human Gene Atlas. (C) Network analysis indicating association between gene ontology terms in (B) and the differentially expressed genes.

FIG. 9 shows karyotype analysis of hiTSCs and hbdTSCs. Two hbdTSC lines, hbdTSC#2 and hbdTSC#9, and four hiTSC clones, hiTSC#1, hiTSC#2, hiTSC#4 and hiTSC#11, were subjected to karyotyping analysis using Affymetrix CytoScan 750K array. 50% of hbdTSC lines and 50% of hiTSC lines harbor an intact karyotype. The other 50% of the colonies exhibited few aberrations in a small fraction of the cells. The specific aberrations and the relevant affected fraction of the cells are marked below each plot.

FIGS. 10A-F demonstrate that hiTSCs differentiate into ST-like cells and EVT-like cells. (A) qPCR analysis of relative mRNA levels of ST markers, ERVFRD-1, CSH1, SDC1, CGB and PSG1, and EVT markers NOTCH1, HLA-G and MMP2 across five days in BDM. Results presented as fold change relative to highest expressing day of each colony and normalized to housekeeping control gene GAPDH. Bars indicate standard deviation between technical duplicates in a typical experiment. (B) Flow cytometry analysis of propidium iodide (PI) nuclear-stained cells at day 0, 4 and 8 after switching to BDM in hiTSC#1, indicating spontaneous differentiation and formation of multinucleated syncytia. (C) Bright field image of hiTSC#2 after 6 days in medium for directed differentiation of TSCs into syncytiotrophoblast (STM). (D) qPCR analysis of relative mRNA levels of ST marker genes PSG1, CHSY1 and ERVFRD-1 at days 0, 2 and 6 in STM. Results presented as fold change relative to highest expressed day of each colony and normalized to mRNA of GAPDH gene. (E) Immunofluorescent staining for DAPI, epithelial-specific protein CDH1 and pan-trophoblast marker KRT7 in PFA-fixated undifferentiated hiTSC#2 cells and following 6 days of ST differentiation in STM. (F) Bright filed and Immunofluorescent staining for ST markers CSH1 and SDC1 in PFA-fixated ST cells originating from hiTSC#2 after 6 days of differentiation in STM.

FIG. 11 demonstrates that hiTSCs engrafted into NOD-SCID mice form trophoblastic lesions. (Left) Lesion extracted from NOD-SCID mice after subcutaneous injection of hiTSC cells. For each lesion, approximately 4×10⁶ were subcutaneously injected into NOD-SCID mice. Lesions were collected nine days after injection. (Right) Stained sections of trophoblastic lesions extracted from NOD-SCID mice. Hematoxylin and eosin staining (middle), KRT7 immunohistochemical staining with hematoxylin counter staining (right).

FIG. 12 demonstrates that ectopic expression of GATA3, OCT4, KLF4, KLF5 and MYC convert human fibroblasts into trophoblast stem-like cells. Elderly fibroblasts were transduced with GATA3, OCT4, KLF4, KLF5 and MYC and reprogrammed for 28 days followed by 10 days of dox removal. Shown are representative images demonstrating the morphology of the parental fibroblasts (left) and various hiTSC colonies (right) that emerged following the reprogramming process.

### DESCRIPTION OF SPECIFIC EMBODIMENTS OF THE INVENTION

The present invention, in some embodiments thereof, relates to a method for reprogramming human cells and, more particularly, but not exclusively, to a method for reprogramming human cells to induced trophoblast stem cells (iTSC) or to rejuvenate cells.

Before explaining at least one embodiment of the invention in detail, it is to be understood that the invention is not necessarily limited in its application to the details set forth in the following description or exemplified by the Examples. The invention is capable of other embodiments or of being practiced or carried out in various ways.

Regenerative medicine is a new and expanding discipline that aims at replacing lost or damaged cells, tissues or organs in the human body through cellular transplantation. The generation of induced stem cells and the direct conversion approach provide an invaluable resource of cells for regenerative medicine and disease modeling. In here, the direct conversion approach refers to both de-differentiation of a somatic cell and reprogramming of a stem cell. In mammals, specialized cell types of the placenta mediate the physiological exchange between the fetus and mother during pregnancy. The precursors of these differentiated cells are trophoblast stem cells (TSCs) and therefore, TSCs have tremendous biomedical relevance.

Whilst reducing embodiments of the present invention to practice, the present inventors have now uncovered that transient ectopic expression of TSC key master regulators in human cells leads to the formation of stable and transgene-independent iTSCs that resemble endogenous TSCs in their transcriptome, methylome and function.

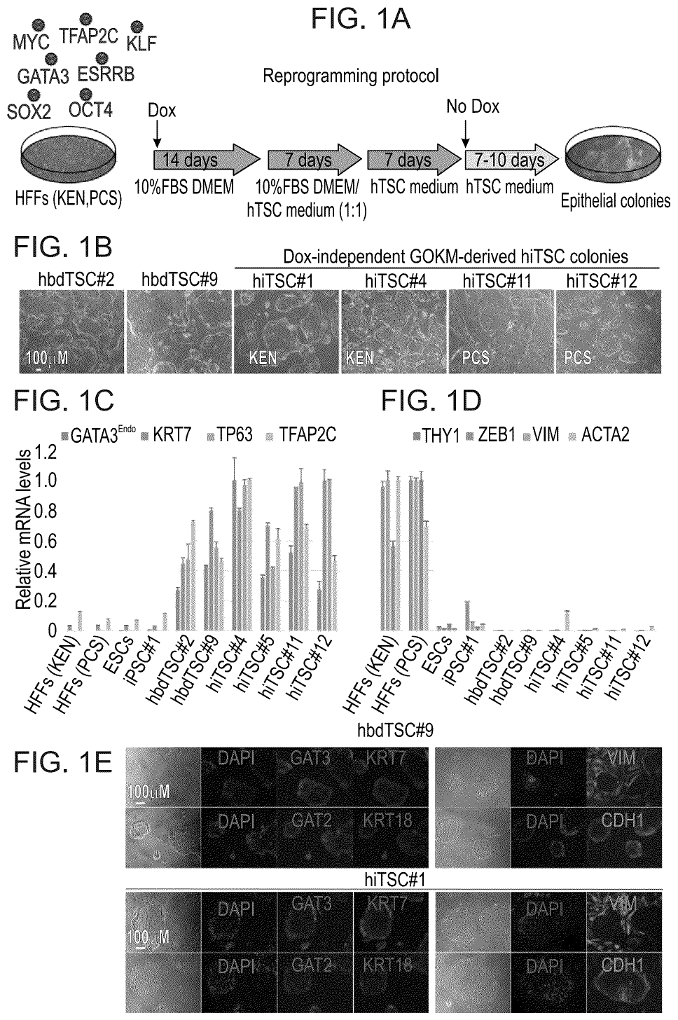

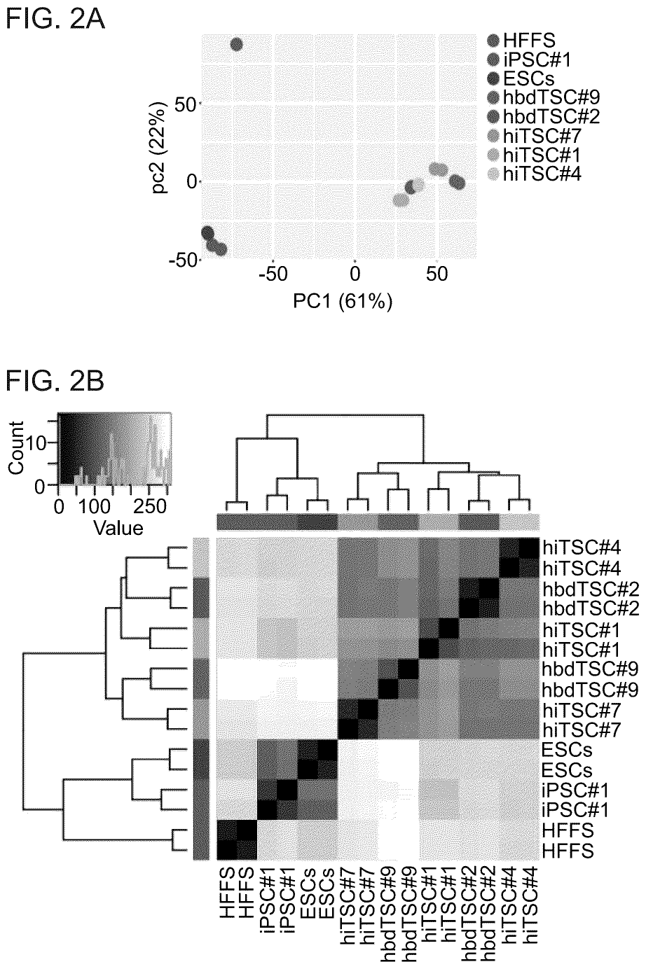

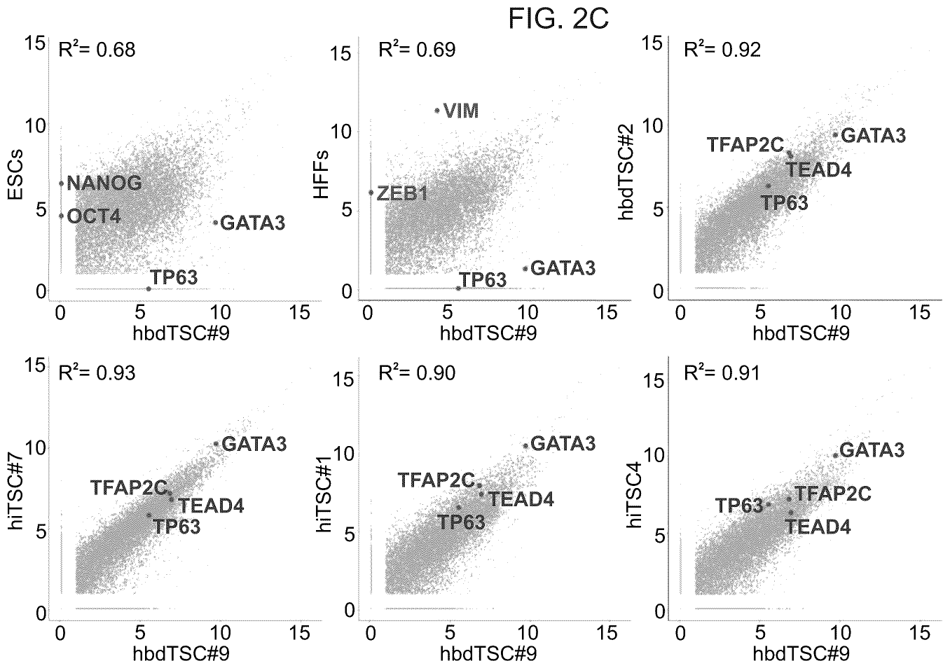

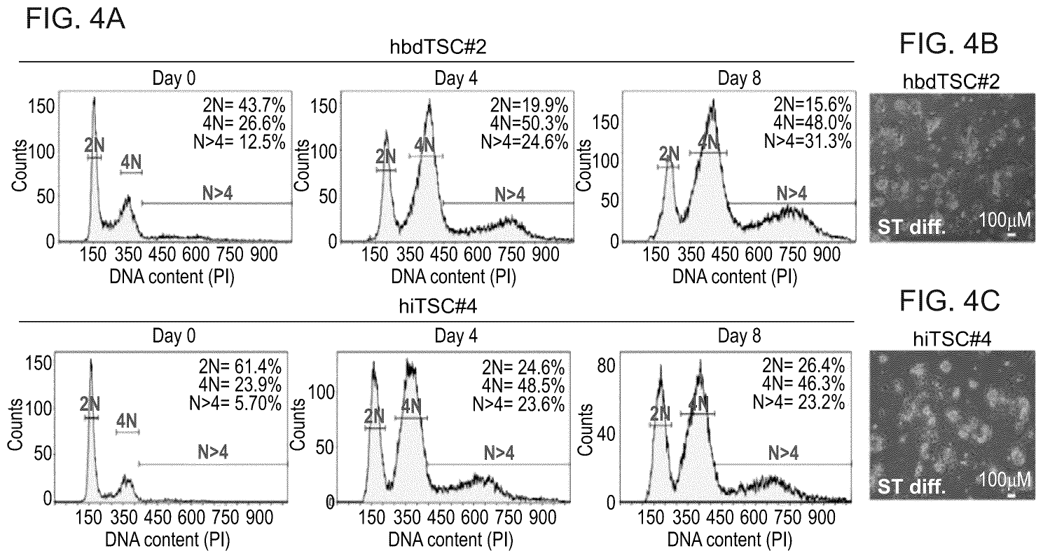

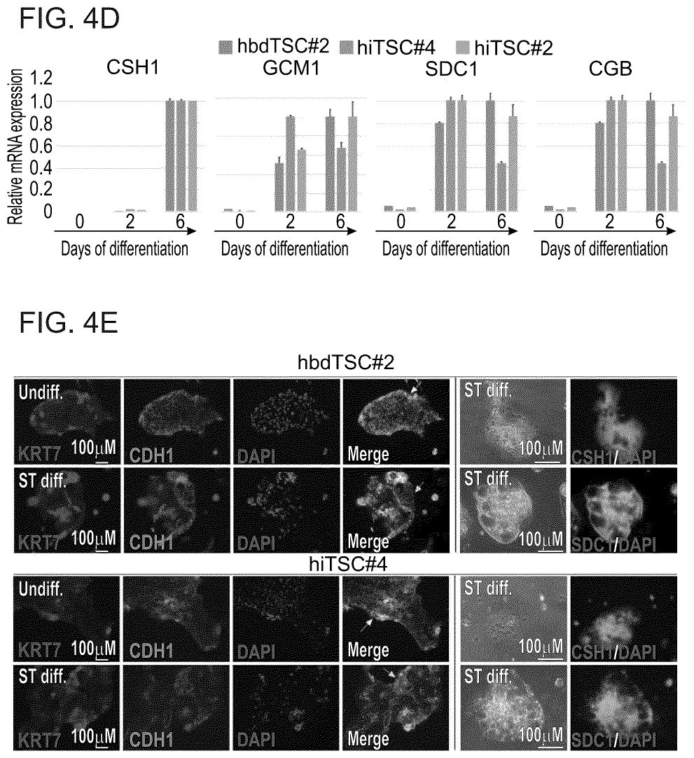

As is illustrated hereinunder and in the examples section, which follows, the present inventors have shown that transient ectopic expression of factors including GATA3 and OCT4 in human fibroblasts, initiates a reprogramming process that leads to the formation of stable and transgene-independent induced trophoblast stem cells (iTSCs) (Examples 1 and 7, FIGS. 1A-E, 7A-G and 12). The induced TSCs may be cultured independently of the exogenous factors for a large number of passages (>20 passages) and resemble blastocyst-derived TSCs in their morphology, genomic integrity, expression of TSC specific markers, no expression of ESC specific and fibroblast specific markers, transcriptome and methylation status (Example 2, FIGS. 2A-C, 3A-D, 8A-C and 9). The inventors further demonstrate that the generated iTSCs can differentiate into syncytium trophoblasts (STs) and extravillous trophoblasts (EVTs), to form trophoblastic lesions in NOD/SCID mice and to form functional organoids in matrigel (Examples 3-5, FIGS. 4A-6C and 10A-11), suggesting that iTSCs acquire all hallmarks of TSCs.

Consequently, specific embodiments suggest the use of GATA3, OCT4 and optionally KLF4, KLF5 and/or c-MYC for generation of iTSC from somatic human cells and their further use in e.g. regenerative medicine, disease modeling, drug screening, and placenta augmentation. Furthermore, this is the first time that an isolated human iTSC was generated that maintained its differentiation level in culture for prolong periods of time (>20 passages) without expressing the exogenous transcription factors used to reprogram the parental cell (i.e. GATA3, OCT4, KLF4 and c-MYC).

Thus, according to an aspect of the present invention, there is provided an isolated human induced trophoblast stem cell (iTSC) comprising an ectopic DNA of GATA3 and OCT4 transcription factors integrated in the genome.

According to specific embodiments, the isolated iTSC comprises an ectopic DNA of a c-MYC transcription factor integrated in the genome.

According to specific embodiments, the isolated iTSC comprises an ectopic DNA of a KLF transcription factor integrated in the genome.

According to specific embodiments, the isolated iTSC comprises an ectopic DNA of a KLF4 transcription factor integrated in the genome.

According to specific embodiments, the isolated iTSC comprises an ectopic DNA of a KLF5 transcription factor integrated in the genome.

According to specific embodiments, the isolated iTSC comprises an ectopic DNA of at least one of KLF4 and KLF5 transcription factors integrated in the genome. According to specific embodiments, the isolated iTSC comprises an ectopic DNA of KLF4 and KLF5 transcription factors integrated in the genome.

As used herein the term “isolated” refers to at least partially separated from the natural environment e.g., from the mammalian (e.g., human) embryo or the mammalian (e.g., human) body or from other cells in culture.

According to specific embodiments, isolation can be done such that pure populations e.g., above 80%, above 85%, above 90%, above 95% or 100% iTSCs, rejuvenated cells or de-differentiated cells are produced.

As used herein the term “induced trophoblast stem cell (iTSC)” refers to a cell obtained by de-differentiation or re-programming of a cell. The iTSC thus produced is endowed with multipotency, in this case being capable of differentiating into the trophoblastic lineage. According to specific embodiments, such cells are obtained from a differentiated cell (e.g. a somatic cell such as a fibroblast) and undergo de-differentiation by genetic manipulation which re-program the cell to acquire trophoblast stem cells (TSC) characteristics. According to specific embodiments, the iTSC is capable of differentiating to the three types of the trophoblast lineage cells in the placental tissue: the villous cytotrophoblast, the syncytiotrophoblast, and the extravillous trophoblast. The villous cytotrophoblast cells are specialized placental epithelial cells which differentiate, proliferate and invade the uterine wall to form the villi. Cytotrophoblasts, which are present in anchoring villi, can fuse to form the syncytiotrophoblast layer or form columns of extravillous trophoblasts (Cohen S. et al., 2003. J. Pathol. 200: 47-52).

According to specific embodiments, the iTSC is a human cell.

An iTSC is typically similar to a TSC which is derived from the placenta of a mammalian embryo in e.g. morphology, expression of specific markers, transcriptome, methylation pattern, and function, as further described below.

According to specific embodiments, the iTSC is characterized by at least one of:
(i) TSC morphology, as determined by e.g. microscopic evaluation (by bright field or H&E staining, electron microscopy. According to specific embodiments the TSC morphology is characterized by flat dense colony with higher edges;
(ii) TSC markers, as determined by an immunocytochemistry and/or PCR assay;
(iii) absence of fibroblast specific markers, as determined by an immunocytochemistry and/or PCR assay;
(iv) a transcriptome similar to a blastocyst-derived TSC, as determined by a RNA sequencing assay;
(v) genomic stability similar to a blastocyst-derived TSC, as determined by a Chromosomal Microarray Analysis;
(vi) a methylation pattern similar to a blastocyst-derived TSC, as determined by a bisulfate assay;
(vii) in-vitro differentiation following culture in a medium without factors supporting an undifferentiated state (e.g. when cultured in DMEM medium with 10% FBS) or in a medium conducive to directed differentiation (e.g. as described in Okae et al. Cell Stem Cell. 2018 Jan. 4; 22(1):50-63 or Haider et al. Stem Cell Reports. 2018 Aug. 14; 11(2):537-551, the contents of which are fully incorporated herein by reference), as determined by morphology, flow cytometry and/or PCR assay;
(viii) in-vitro and/or in-vivo differentiation into derivatives of the trophectoderm lineage, as determined by morphology, immunocytochemistry, immunocytochemistry, flow cytometry and/or PCR assay;
(ix) Ability to form a three dimensional organoid culture (e.g. such as described in Haider et al. Stem Cell Reports. 2018 Aug. 14; 11(2):537-551, the contents of which are fully incorporated herein by reference), as determined by morphology, immunocytochemistry, immunocytochemistry and/or PCR assay;
(x) in-vivo formation of a trophoblastic lesion (e.g. in NOD/SCID mice), as determined by histological evaluation; and
(xi) no change in differentiation level for at least 20 passages in culture as determined by at least one of the assay in (i-)-(x).

According to specific embodiments, the TSC markers are selected from the group consisting of KRT7, GATA2, GATA3, TFAP2A, TFAP2C, TP63.

According to specific embodiments, the ESC specific markers are selected from the group consisting of NANOG, OCT4, SOX2.

According to specific embodiments, the mesenchymal markers are selected from the group consisting of THY1, ZEB1, VIM, ACTA2.

According to specific embodiments, the methylation pattern comprises hypomethylation of the ELF5 promoter area and/or hypermethylation of the Nanog promoter as compared to the parental non-reprogrammed cell and/or an ESC cell.

According to specific embodiments the iTSC is characterized by absence of embryonic stem cell (ESC) specific markers (e.g. NANOG, OCT4 and SOX2), as determined by an immunocytochemistry and/or PCR assay.

According to specific embodiments, the iTSC maintains differentiation level of a TSC for at least 20, at least 30, at least 50 passages in culture.

According to a specific embodiment, the iTSC maintains its differentiation level of a TSC for at least 20 passages.

According to other specific embodiments, the iTSC maintains differentiation level of a TSC in an absence of expression of an exogenous transcription factor as determined by e.g. a PCR assay.

According to specific embodiments, the iTSC does not express the exogenous transcription factor as determined by PCR, western blot and/or flow cytometry.

According to a specific embodiment, the iTSC does not express exogenous GATA3, OCT4, KLF and c-MYC transcription factors as determined by PCR, western blot and/or flow cytometry.

According to a specific embodiment, the iTSC does not express exogenous GATA3, OCT4, KLF4 and c-MYC transcription factors as determined by PCR, western blot and/or flow cytometry.

According to a specific embodiment, the iTSC does not express exogenous GATA3, OCT4, KLF4, KLF5 and c-MYC transcription factors as determined by PCR, western blot and/or flow cytometry.

According to specific embodiments, the iTSC expresses an exogenous transcription factor not in the natural location (i.e., gene locus) and/or expression level (e.g., copy number and/or cellular localization) of the native gene of the transcription factor.

According to specific embodiments, the iTSC comprises an ectopic DNA of an exogenous transcription factor integrated in the genome of the cell but not in its natural location (i.e. locus) and/or copy number.

According to specific embodiments, the transcription factor is selected form the group consisting of GATA3, OCT4, KLF and c-MYC.

According to specific embodiments, the transcription factor is selected form the group consisting of GATA3, OCT4, KLF4 and c-MYC.

According to specific embodiments, the transcription factor is selected form the group consisting of GATA3, OCT4, KLF4, KLF5 and c-MYC.

As described, the present inventors have developed a novel method for generating a human iTSC.

Thus, according to an additional or an alternative aspect of the present invention, there is provided a method of generating an induced trophoblast stem cell (iTSC) from a human cell, the method comprising expressing within the cell exogenous GATA3 and OCT4 transcription factors, under conditions which allow generation of an iTSC from said cell, thereby generating the iTSC from the cell.

According to specific embodiments, the method further comprising expressing within said cell an exogenous c-MYC transcription factor.

According to specific embodiments, the method further comprising expressing within said cell an exogenous KLF transcription factor.

According to specific embodiments, the method further comprising expressing within said cell an exogenous KLF4 transcription factor.

According to specific embodiments, the method further comprising expressing within said cell an exogenous KLF5 transcription factor.

According to specific embodiments, the method further comprising expressing within said cell at least one of exogenous KLF4 and KLF5 transcription factors.

According to specific embodiments, the method further comprising expressing within said cell exogenous KLF4 and KLF5 transcription factors.

According to an aspect of some embodiments of the invention, there is provided an isolated human induced trophoblast stem cell (iTSC) obtainable by the method disclosed herein.

Further, as the expression of the TSC key master regulators disclosed herein induced reprogramming of somatic cells to multipotent cells; specific embodiments suggest the use of GATA3, OCT4 and optionally KLF (e.g. KLF4, KLF5, KLF6, KLF15) and/or c-MYC for de-differentiating cells.

In addition, epigenetic alterations, such as DNA methylation, have been proposed as a major cause of age-related diseases such as cognitive decline and cardiovascular disorders. As the expression of the TSC key master regulators disclosed herein induced reprogramming of somatic cells to multipotent cells while affecting DNA methylation to thereby revert the epigenetic clock; specific embodiments suggest the use of GATA3, OCT4 and optionally KLF (e.g. KLF4, KLF5, KLF6, KLF15) and/or c-MYC for rejuvenating elderly cells.

Thus, according to an additional or an alternative aspect of the present invention, there is provided a method of rejuvenating and/or de-differentiating a human cell, the method comprising expressing within the cell exogenous GATA3 and OCT4 transcription factors, under conditions which allow rejuvenation and/or de-differentiation of said cell, thereby generating a rejuvenated cell and/or a de-differentiated cell.

According to specific embodiments, the method further comprising expressing within said cell an exogenous c-MYC transcription factor.

According to specific embodiments, the method further comprising expressing within said cell an exogenous KLF transcription factor.

According to specific embodiments, the method further comprising expressing within said cell an exogenous KLF4 transcription factor.

According to specific embodiments, the method further comprising expressing within said cell an exogenous KLF5 transcription factor.

According to specific embodiments, the method further comprising expressing within said cell at least one of exogenous KLF4 and KLF5 transcription factors.

According to specific embodiments, the method further comprising expressing within said cell exogenous KLF4 and KLF5 transcription factors.

According to an aspect of some embodiments of the invention, there is provided an isolated human rejuvenated and/or de-differentiated cell obtainable by the method disclosed herein.

As used herein, the term “de-differentiated cell” refers to a cell obtained by de-differentiation or re-programming of a cell to become less specialized and return to an earlier developmental state within the same lineage. Methods of determining de-differentiation are known in the art and are further described hereinbelow and include, but not limited to, morphology assessment, expression of markers, in-vitro and/or in-vivo differentiation as compared to the cell it is derived from (i.e. a cell of the same type that has not been subjected to the method).

As used herein, the term “elderly cell” refers to a cell derived from an adult organism e.g. at least 20 years old human subject.

As used herein, the term “rejuvenated cell” refers to a cell of the same lineage and differentiation/developmental state as the cell it is derived from but with a younger age characteristics, which may be determined by e.g. the epigenetic signature.

According to specific embodiments, the rejuvenated cell has an improved function as compared to the cell it is derived from.

According to specific embodiments, the rejuvenated cell is characterized by at least one of:
(i) a morphology of a cell of the same type and developmental stage that has not been subjected to said method;
(ii) markers of a cell of the same type and developmental stage that has not been subjected to said method, as determined by an immunostaining, Western blot and/or PCR assay;
(iii) a transcriptome, with the exception of genes that are associated with age, similar to a cell of the same type and developmental stage that has not been subjected to said method, with the exception of genes that as determined by a RNA sequencing assay;
(iv) a methylation pattern distinct from a cell of the same type and developmental stage that has not been subjected to said method, as determined by a bisulfate assay.

According to specific embodiments, the rejuvenated or de-differentiated cell does not express the exogenous transcription factor as determined by PCR, western blot and/or flow cytometry.

According to a specific embodiment, the rejuvenated cell or de-differentiated does not express exogenous GATA3, OCT4, KLF and c-MYC transcription factors as determined by PCR, western blot and/or flow cytometry.

According to a specific embodiment, the rejuvenated cell or de-differentiated does not express exogenous GATA3, OCT4, KLF4 and c-MYC transcription factors as determined by PCR, western blot and/or flow cytometry.

According to a specific embodiment, the rejuvenated cell or de-differentiated does not express exogenous GATA3, OCT4, KLF4, KLF5 and c-MYC transcription factors as determined by PCR, western blot and/or flow cytometry.

According to an aspect of some embodiments of the invention, there is provided an isolated human rejuvenated cell obtainable by the method disclosed herein.

According to an aspect of some embodiments of the invention, there is provided an isolated human de-differentiated cell obtainable by the method disclosed herein.

As used herein the term “cell” refers to any cell derived from an organism including an adult cell, a fetal cell, a somatic cell and a stem cell.

According to specific embodiments, the cell is an elderly cell.

According to specific embodiments, the cell is a stem cell.

As used herein, the phrase “stem cell” refers to a cell which is not terminally differentiated i.e., capable of differentiating into other cell types having a more particular, specialized function (e.g., fully differentiated cells). The term encompasses embryonic stem cells, fetal stem cells, adult stem cells or committed/progenitor cells.

According to specific embodiments, the cell is a somatic cell.

As used herein, the phrase “somatic cell” refers to a terminally differentiated cell. Non-limiting examples of somatic cells include a fibroblast, a blood cell, an endothelial cell, a hepatocyte, a pancreatic cell, a cartilage cell, a myocyte, a cardiomyocyte, a smooth muscle cell, a keratinocyte, a neural cell, a retinal cell, an epidermal cell, an epithelial cell (e.g., isolated from the oral cavity) or a cell isolated from placenta.

According to specific embodiments, the somatic cell is selected from the group consisting of a fibroblast, a blood cell, a keratinocyte, an epithelial cells e.g., a cell isolated from the oral cavity or a cell isolated from placenta.

According to a specific embodiment, the somatic cell is a fibroblast.

According to specific embodiments, the cell is selected from the group consisting of keratinocyte, hematopoietic cell, retinal cell (e.g. PE, Photoreceptor), fibroblast, hepatocyte, cardiac cell, kidney cell, pancreatic cell (e.g. alpha, beta) and neuron.

According to specific embodiments, the cell is a hematopoietic cell or mesenchymal stem cell. According to specific embodiments, the cell is a human cell.

According to specific embodiments, the cell is comprised in a homogenous population of cells, e.g. wherein at least about 80% of the cells in the population are iTSCs, rejuvenated cells or de-differentiated cells.

Thus, according to an aspect of the present invention, there is provided an isolated population of cells, wherein at least 80%, at least 85%, at least 90%, at least 95%, at least 97%, at least 98% of the cells are the iTSCs disclosed herein.

According to other specific embodiments, the cell is comprised in a heterogeneous population of cells, i.e. in a population which comprises more than one cell type, e.g. in which at least 5%, at least 10%, at least 15%, at least 20%, at least 30% are iTSCs.

According to an additional or an alternative aspect of the present invention, there is provided an isolated population of cells, wherein at least 80%, at least 85%, at least 90%, at least 95%, at least 97%, at least 98% of the cells are the rejuvenated cells disclosed herein.

According to other specific embodiments, the cell is comprised in a heterogeneous population of cells, i.e. in a population which comprises more than one cell type, e.g. in which at least 5%, at least 10%, at least 15%, at least 20%, at least 30% are rejuvenated cells.

According to an additional or an alternative aspect of the present invention, there is provided an isolated population of cells, wherein at least 80%, at least 85%, at least 90%, at least 95%, at least 97%, at least 98% of the cells are the de-differentiated cells disclosed herein.

According to other specific embodiments, the cell is comprised in a heterogeneous population of cells, i.e. in a population which comprises more than one cell type, e.g. in which at least 5%, at least 10%, at least 15%, at least 20%, at least 30% are de-differentiated cells.

As mentioned, an exogenous transcription factor is expressed in the cell.

As used herein, the term “transcription factor” refers to a cellular factor regulating gene transcription. According to specific embodiments, the transcription factor is a polypeptide with the ability to bind a specific nucleic acid sequence (i.e. the binding site) which is specific for a specific transcription factor(s). Non-limiting examples of transcription factors include GATA3, OCT4, KLF (e.g. KLF4, KLF5, KLF6, KLF15) and c-MYC.

As used herein, the term “GATA3”, also known as GATA Binding Protein 3 and HDRS, refers to the polynucleotide and expression product e.g., polypeptide of the GATA3 gene. According to specific embodiments the GATA3 refers to the human GATA3, such as provided in the following GeneBank Numbers NP 001002295 and NM 001002295 (SEQ ID NO: 1-2). A functional expression product of GATA3 is capable of supporting, optionally along with other factors which are described herein, the generation of iTSC.

As used herein, the term “OCT4 (octamer-binding transcription factor 4)”, also known as POU5F1, refers to the polynucleotide and expression product e.g., polypeptide of the POU5F1 gene. According to specific embodiments the OCT4 refers to the human OCT4, such as provided in the following GeneBank Numbers NP_001167002, NP_001272915, NP_001272916, NP_002692, NP_976034, NM_203289, NM_001173531, NM_001285986, NM_001285987 and NM_002701 (SEQ ID NO: 3-12). A functional expression product of OCT4 is capable of supporting, optionally along with other factors, which are described herein, the generation of iTSC.

As used herein, the term “KLF”, refers to the polynucleotide and expression product e.g., polypeptide of any one of the Kruppel-like family of transcription factors, which are a set of C2H2 zinc finger DNA-binding proteins that regulate gene expression. According to specific embodiments the KLF refers to the human KLF. A functional expression product of KLF is capable of supporting, optionally along with other factors, which are described herein, the generation of iTSC. Non-limiting examples of KLF transcription factors include KLF1, KLF2, KLF3, KLF4, KLF5, KLF6, KLF7, KLF8, KLF9, KLF10, KLF11, KLF12, KLF13, KLT14, KLF15, KLF16, KLF17.

According to specific embodiments, the KLF transcription factor comprises at least one of the KLF transcription factors.

According to specific embodiments, the KLF transcription factor is selected from the group consisting of KLF4, KLF5, KLF6 and KLF15.

According to specific embodiments, the KLF transcription factor is selected from the group consisting of KLF4 and KLF5.

According to specific embodiments, the at least one of KLF transcription factors comprises at least two distinct KLF transcription factors.

According to specific embodiments, the KLF transcription factor comprises both KLF4 and KLF5.

According to specific embodiments, the KLF transcription factor comprises KLF4.

As used herein, the term “KLF4 (Kruppel-like factor 4)”, also known as GKLF and EZF refers to the polynucleotide and expression product e.g., polypeptide of the KLF4 gene. According to specific embodiments the KLF4 refers to the human KLF4, such as provided in the following GeneBank Numbers NP_004226 and NM_004235 (SEQ ID NO: 13-14). A functional expression product of KLF4 is capable of supporting, optionally along with other factors, which are described herein, the generation of iTSC.

According to specific embodiments, the KLF transcription factor comprises KLF5.

As used herein, the term “KLF5 (Kruppel-like factor 5)”, also known as BTEB2, CKLF and IKLF refers to the polynucleotide and expression product e.g., polypeptide of the KLF5 gene. According to specific embodiments the KLF5 refers to the human KLF5, such as provided in the following GeneBank Numbers NP_001273747, NP_001721, NM_001730 and NM_001286818 (SEQ ID NO: 81-84). A functional expression product of KLF5 is capable of supporting, optionally along with other factors, which are described herein, the generation of iTSC.

According to specific embodiments, the KLF transcription factor comprises KLF6.

As used herein, the term “KLF6 (Kruppel-like factor 6)”, also known as BCD1, CBA1, COPEB, PAC1, ST12 and ZF9 refers to the polynucleotide and expression product e.g., polypeptide of the KLF6 gene. According to specific embodiments the KLF6 refers to the human KLF6, such as provided in the following GeneBank Numbers NP_001153596, NP_001153597, NP_001291, NM_001008490, NM_001160124, NM_001160125 and NM_001300 (SEQ ID NO: 85-91). A functional expression product of KLF6 is capable of supporting, optionally along with other factors, which are described herein, the generation of iTSC.

According to specific embodiments, the KLF transcription factor comprises KLF15.

As used herein, the term “KLF15 (Kruppel-like factor 615”, refers to the polynucleotide and expression product e.g., polypeptide of the KLF15 gene. According to specific embodiments the KLF15 refers to the human KLF15, such as provided in the following GeneBank Numbers NP_054798, NM_014079 (SEQ ID NO: 92-93). A functional expression product of KLF15 is capable of supporting, optionally along with other factors, which are described herein, the generation of iTSC.

As used herein, the term “c-MYC” also known as V-Myc Avian Myelocytomatosis Viral Oncogene Homolog, Class E Basic Helix-Loop-Helix Protein 39, Transcription Factor P64, BHLHe39, MRTL and MYCC, refers to the polynucleotide and expression product e.g., polypeptide of the MYC gene. According to specific embodiments, the c-MYC refers to the human c-MYC, such as provided in the following GeneBank Numbers NP_002458 and NM_002467 (SEQ ID NO: 15-16). A functional expression product of c-MYC is capable of supporting, along with other factors, which are described herein, the generation of iTSC.

The terms “GATA3”, “OCT4”, “KLF4”, “KLF” and “c-MYC”, also refer to functional GATA3, OCT4, KLF (e.g. KLF4, KLF5, KLF6, KLF15) and c-MYC homologues which exhibit the desired activity (i.e., de-differentiating or reprogramming a cell to an iTSC). Such homologues can be, for example, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% identical or homologous to the polypeptide of SEQ ID NOs: 1, 3-7, 13, 81-93 and 15, respectively, or 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% identical to the polynucleotide sequence encoding same (as further described hereinbelow).

The homolog may also refer to an ortholog, a deletion, insertion, or substitution variant, including an amino acid substitution, as long as it retains the activity.

Sequence identity or homology can be determined using any protein or nucleic acid sequence alignment algorithm such as Blast, ClustalW, and MUSCLE.

Specific embodiments of the present invention contemplate expressing at least GATA3 and OCT4 and optionally c-MYC and/or KLF transcription factors.

Specific embodiments of the present invention contemplate expressing at least GATA3 and OCT4 and optionally c-MYC, KLF4 and/or KLF5 transcription factors.

Specific embodiments of the present invention contemplate expressing at least GATA3 and OCT4 and optionally c-MYC and/or KLF4 transcription factors.

According to specific embodiments, two, three or all of the transcription factors are exogenously expressed in the cell e.g.: GATA3+OCT4, GATA3+OCT4+c-MYC, GATA3+OCT4+KLF (e.g. GATA3+OCT4+KLF4, GATA3+OCT4+KLF5, GATA3+OCT4+KLF4+KLF5), GATA3+OCT4+c-MYC+KLF (e.g. GATA3+OCT4+c-MYC+KLF4, GATA3+OCT4+c-MYC+KLF5, GATA3+OCT4+c-MYC+KLF4+KLF5).

According to specific embodiments all of the transcription factors are exogenously expressed in the cell i.e. GATA3+OCT4+c-MYC+KLF (e.g. GATA3+OCT4+c-MYC+KLF4+KLF5).

As used herein, the term “expressing” or “expression” refers to gene expression at the RNA and/or protein level. The term also refers to upregulating gene expression by expressing the DNA or RNA or upregulating the level of the protein by direct administration of the protein to the cell.

As used herein, the term “exogenous” refers to a heterologous polynucleotide or polypeptide which is not naturally expressed within the cell or which overexpression in the cell is desired. The exogenous polynucleotide and/or polypeptide may be introduced into the cell in a stable or transient manner. In the case of a polynucleotide introduction is effected so as to produce a ribonucleic acid (RNA) molecule and/or a polypeptide molecule. According to specific embodiments, expressing comprises transiently expressing. It should be noted that the exogenous polynucleotide and/or polypeptide may comprise a nucleic acid sequence and/or an amino acid sequence, respectively, which is identical or partially homologous to an endogenous nucleic acid sequence and/or an endogenous amino acid sequence of the cell. Methods of expressing an exogenous nucleic acid sequence and/or amino acid sequence are known in the art and include those described for example in the materials and methods of the Examples section which follows and in Mansour et al. 2012; Warren et al. 2010 and Hongyan Zhou al. Cell Stem Cell (2009) 4(6): 581; Rabinovich and Weissman (2013) Methods Mol Biol. 969:3-28; International Application Publication No. WO 2013049389 and U.S. Pat. No. 8,557,972, which are fully incorporated herein by reference in their entirety.

Further description of preparation of expression vectors and modes of administering them into cells are provided hereinunder.

According to specific embodiments, expressing is not in the natural location (i.e., gene locus) and/or expression level (e.g., copy number and/or cellular localization) of the native gene of the transcription factor.

According to other specific embodiments, expressing is not in the natural position and/or copy number of the native gene of the transcription factor in a genome.

Alternatively or additionally, exogenous expression of a transcription factor may be facilitated by activation of the endogenous locus of these genes such that the transcription factor is overexpressed in the cell. Methods of activating and overexpressing an endogenous gene are well known in the art [see for example Menke D. Genesis (2013) 51:-618; Capecchi, Science (1989) 244:1288-1292; Santiago et al. Proc Natl Acad Sci USA (2008) 105:5809-5814; International Patent Application Nos. WO 2014085593, WO 2009071334 and WO 2011146121; U.S. Pat. Nos. 8,771,945, 8,586,526, 6,774,279 and UP Patent Application Publication Nos. 20030232410, 20050026157, US20060014264; the contents of which are incorporated by reference in their entireties] and include, but not limited to and include targeted homologous recombination (e.g. “Hit and run”, “double-replacement”), site specific recombinases (e.g. the Cre recombinase and the Flp recombinase), PB transposases (e.g. Sleeping Beauty, piggyBac, Tol2 or Frog Prince), genome editing by engineered nucleases (e.g. meganucleases, Zinc finger nucleases (ZFNs), transcription-activator like effector nucleases (TALENs) and CRISPR/Cas system) and genome editing using recombinant adeno-associated virus (rAAV) platform, and small molecules. Agents for introducing nucleic acid alterations to a gene of interest can be designed publically available sources or obtained commercially from Transposagen, Addgene and Sangamo Biosciences.

The term “endogenous” as used herein refers to a polynucleotide or polypeptide which is present and/or naturally expressed within the cell.

Distinguishing a cell expressing an exogenous polynucleotide and/or polypeptide (e.g. transcription factor) from a cell not expressing the exogenous polynucleotide and/or polypeptide can be effected by e.g. determining the level and/or distribution of the RNA and/or protein molecules in the cell, the location of DNA integration in the genome of the cell and/or the number of gene copy number. Methods of determining the presence of an exogenous polynucleotide and/or polypeptide in a cell are well known in the art and include e.g. PCR, DNA and RNA sequencing, Southern blot, Western blot, immunoprecipitation, immunocytochemistry, flow cytometry and imaging.

As used herein the term “polynucleotide” refers to a single or double stranded nucleic acid sequence in the form of an RNA sequence (e.g. mRNA), a complementary polynucleotide sequence (cDNA), a genomic polynucleotide sequence (e.g. sequence isolated from a chromosome), a composite polynucleotide sequences (e.g., a combination of the above) or mimetic or analog thereof. This term includes polynucleotides and/or oligonucleotides derived from naturally occurring nucleic acids molecules (e.g., RNA or DNA), synthetic polynucleotide and/or oligonucleotide molecules composed of naturally occurring bases, sugars, and covalent internucleoside linkages (e.g., backbone), as well as synthetic polynucleotides and/or oligonucleotides having non-naturally occurring portions, which function similarly to the respective naturally occurring portions.

According to specific embodiments, the polynucleotide is a modified polynucleotide e.g. modified RNA.

Such a modified polynucleotide may comprise a modification in either backbone, internucleoside linkages or bases. The modified polynucleotide may comprise naturally modified nucleotides or synthetic nucleoside analogous. Modified polynucleotides may be preferred over native forms according to specific embodiments, because of desirable properties such as, for example, enhanced cellular uptake, enhanced affinity for nucleic acid target, increased stability in the presence of nucleases and decreased immunogenicity.

Such modifications include, but are not limited to 5-methoxyuridine, Pseudouridine, 5-methyl cytidine, N6-methyladenosine, 2′-O-methyl, 2′-O-methyl 3′phosphorothioate, 2′-O-methyl 3′thiophosphonoacetate, Locked nucleic acid (LNA).

Methods of stabilizing mRNA are known in the art and include modulation of the length of the polyadenine tail found at the 3″ end of the mRNA transcript. Alternatively or additionally, the RNA cap found at the molecule's 5′ end can be modified. The naturally occurring cap structure typical in mammalian cells has a tendency to be improperly incorporated into RNAs synthesized in vitro, rendering them less effective. Synthetic “anti-reverse cap analogs” (e.g. those commercially available at Thermo Fisher Scientific) can prevent this misincorporation, which results in more stable RNA with improved translational efficiency. In order to reduce immunogenicity, substitution of particular nucleotides can be exchanged with chemically modified alternatives such as 5-methylcytosine or pseudoruidine. Such substitutions can mute the immune response whilst also bolstering the stability of the mRNA and efficiency of translation. Other exemplary chemically modified nucleotides are described herein above.

Alternatively, or additionally, the mRNA may be encapsulated in lipid-based particles to enhance fusion with the lipid cell membrane.

According to specific embodiments, the polynucleotide is an isolated polynucleotide.

Polynucleotides designed according to the teachings of some embodiments of the invention can be generated according to any polynucleotide synthesis method known in the art such as enzymatic synthesis or solid phase synthesis. Equipment and reagents for executing solid-phase synthesis are commercially available from, for example, Applied Biosystems. Any other means for such synthesis may also be employed; the actual synthesis of the polynucleotides is well within the capabilities of one skilled in the art and can be accomplished via established methodologies as detailed in, for example, “Molecular Cloning: A laboratory Manual” Sambrook et al., (1989); “Current Protocols in Molecular Biology” Volumes I-III Ausubel, R. M., ed. (1994); Ausubel et al., “Current Protocols in Molecular Biology”, John Wiley and Sons, Baltimore, Maryland (1989); Perbal, “A Practical Guide to Molecular Cloning”, John Wiley & Sons, New York (1988) and “Oligonucleotide Synthesis” Gait, M. J., ed. (1984) utilizing solid phase chemistry, e.g. cyanoethyl phosphoramidite followed by deprotection, desalting and purification by for example, an automated trityl-on method or HPLC.

The term “polypeptide” or “protein” as used herein encompasses native peptides (either degradation products, synthetically synthesized peptides or recombinant peptides) and peptidomimetics (typically, synthetically synthesized peptides), as well as peptoids and semipeptoids which are peptide analogs, which may have, for example, modifications rendering the peptides more stable while in a body or more capable of penetrating into cells. Such modifications include, but are not limited to N terminus modification, C terminus modification, peptide bond modification, backbone modifications, and residue modification. Methods for preparing peptidomimetic compounds are well known in the art and are specified, for example, in Quantitative Drug Design, C. A. Ramsden Gd., Chapter 17.2, F. Choplin Pergamon Press (1992), which is incorporated by reference as if fully set forth herein.

Peptide bonds (—CO—NH—) within the peptide may be substituted, for example, by N-methylated amide bonds (—N(CH3)-CO—), ester bonds (—C(═O)—O—), ketomethylene bonds (—CO—CH2-), sulfinylmethylene bonds (—S(═O)—CH2-), α-aza bonds (—NH—N(R)—CO—), wherein R is any alkyl (e.g., methyl), amine bonds (—CH2-NH—), sulfide bonds (—CH2-S—), ethylene bonds (—CH2-CH2-), hydroxyethylene bonds (—CH(OH)—CH2-), thioamide bonds (—CS—NH—), olefinic double bonds (—CH═CH—), fluorinated olefinic double bonds (—CF═CH—), retro amide bonds (—NH—CO—), peptide derivatives (—N(R)—CH2-CO—), wherein R is the “normal” side chain, naturally present on the carbon atom.

These modifications can occur at any of the bonds along the polypeptide chain and even at several (2-3) bonds at the same time.

Natural aromatic amino acids, Trp, Tyr and Phe, may be substituted by non-natural aromatic amino acids such as 1,2,3,4-tetrahydroisoquinoline-3-carboxylic acid (Tic), naphthylalanine, ring-methylated derivatives of Phe, halogenated derivatives of Phe or O-methyl-Tyr.

The polypeptides of some embodiments of the invention may also include one or more modified amino acids or one or more non-amino acid monomers (e.g. fatty acids, complex carbohydrates etc).

The term “amino acid” or “amino acids” is understood to include the 20 naturally occurring amino acids; those amino acids often modified post-translationally in vivo, including, for example, hydroxyproline, phosphoserine and phosphothreonine; and other unusual amino acids including, but not limited to, 2-aminoadipic acid, hydroxylysine, isodesmosine, nor-valine, nor-leucine and ornithine. Furthermore, the term “amino acid” includes both D- and L-amino acids.

The polypeptides of some embodiments of the invention may be synthesized by any techniques known to those skilled in the art of peptide synthesis, for example but not limited to recombinant DNA techniques or solid phase peptide synthesis.

Following is a non-limiting description of expression vectors and modes of administering thereof into cells which can be used to express a polypeptide-of-interest [e.g., any of the proteins described hereinabove and below, e.g. GATA3, OCT4, KLF (e.g. KLF4, KLF5, KLF6, FKL15) and c-MYC] in a cell.

According to specific embodiments, expressing comprises introducing into the cell a polynucleotide encoding the polypeptide-of-interest (e.g. the transcription factor).

According to specific embodiments, the polynucleotide is a DNA.

According to specific embodiments, the polynucleotide is a RNA. Typically, mRNA introduced into cells exists only in the cytoplasm, does not cause genome perturbations and is essentially transient. Unless expression of the mRNA changes the cell epigenetically, transient transfection is limited by the time of mRNA and cognate protein persistence in the cell, and does not continue after degradation of cognate proteins.

To express an exogenous protein in mammalian cells, a polynucleotide sequence encoding the polypeptide-of-interest is preferably ligated into a nucleic acid construct suitable for mammalian cell expression.

Teachings of the invention further contemplate that the polynucleotides are part of a nucleic acid construct system where the polypeptides of interest are expressed from a plurality of constructs.

It will be appreciated that over-expression or exclusion of genes can be effected using knock-in and/or knock-out constructs [see for example, Fukushige, S. and Ikeda, J. E.: Trapping of mammalian promoters by Cre-lox site-specific recombination. DNA Res 3 (1996) 73-50; Bedell, M. A., Jerkins, N. A. and Copeland, N. G.: Mouse models of human disease. Part I: Techniques and resources for genetic analysis in mice. Genes and Development 11 (1997) 1-11; Bermingham, J. J., Scherer, S. S., O'Connell, S., Arroyo, E., Kalla, K. A., Powell, F. L. and Rosenfeld, M. G.: Tst-1/Oct-6/SCIP regulates a unique step in peripheral myelination and is required for normal respiration. Genes Dev 10 (1996) 1751-62].

Thus, according to an aspect of the present invention, there is provided a nucleic acid construct or system comprising at least one polynucleotide comprising a nucleic acid sequence encoding GATA3 and OCT4 transcription factors.

According to specific embodiments, the at least one polynucleotide further comprises a nucleic acid sequence encoding c-MYC transcription factor.

According to specific embodiments, at least one polynucleotide further comprises a nucleic acid sequence encoding a KLF transcription factor.

According to specific embodiments, at least one polynucleotide further comprises a nucleic acid sequence encoding KLF4 transcription factor.

According to specific embodiments, at least one polynucleotide further comprises a nucleic acid sequence encoding KLF5 transcription factor.

According to specific embodiments, at least one polynucleotide further comprises a nucleic acid sequence encoding at least one of KLF4 and KLF5 transcription factors.

According to specific embodiments, at least one polynucleotide further comprises a nucleic acid sequence encoding KLF4 and KLF5 transcription factors.

According to specific embodiments, two, three or all of the transcription factors are encoded by the polynucleotide e.g.: GATA3+OCT4; GATA3+OCT4+c-MYC, GATA3+OCT4+KLF (e.g. GATA3+OCT4+KLF4, GATA3+OCT4+KLF5, GATA3+OCT4+KLF4+KLF5), or GATA3+OCT4+c-MYC+KLF (e.g. GATA3+OCT4+c-MYC+KLF4, GATA3+OCT4+c-MYC+KLF5 GATA3+OCT4+c-MYC+KLF4+KLF5).

According to specific embodiments, the nucleic acid construct or system comprising at least one polynucleotide comprising a nucleic acid sequence encoding GATA3, OCT4, c-MYC and KLF.

According to specific embodiments, the nucleic acid construct or system comprising at least one polynucleotide comprising a nucleic acid sequence encoding GATA3, OCT4, c-MYC and KLF4.

According to specific embodiments, the nucleic acid construct or system comprising at least one polynucleotide comprising a nucleic acid sequence encoding GATA3, OCT4, c-MYC, KLF4 and KLF5.

Thus, according to specific embodiments, the nucleic acid construct system comprises an individual nucleic acid construct for each transcription factor.

According to other specific embodiments, a single construct comprises a number of transcription factors.

Such a nucleic acid construct or system includes at least one cis-acting regulatory element for directing expression of the nucleic acid sequence. Cis-acting regulatory sequences include those that direct constitutive expression of a nucleotide sequence as well as those that direct inducible expression of the nucleotide sequence only under certain conditions. Thus, for example, a promoter sequence for directing transcription of the polynucleotide sequence in the cell in a constitutive or inducible manner is included in the nucleic acid construct. In the case of mRNA, since gene expression from an RNA source does not require transcription, there is no need in a promoter sequence or the additional sequences involved in transcription described hereinbelow.

The nucleic acid construct or system (also referred to herein as an “expression vector”) of some embodiments of the invention includes additional sequences which render this vector suitable for replication and integration in prokaryotes, eukaryotes, or preferably both (e.g., shuttle vectors). In addition, a typical cloning vector may also contain a transcription and/or translation initiation sequence, transcription and/or translation terminator and a polyadenylation signal. By way of example, such constructs will typically include a 5′ LTR, a tRNA binding site, a packaging signal, an origin of second-strand DNA synthesis, and a 3′ LTR or a portion thereof.

Eukaryotic promoters typically contain two types of recognition sequences, the TATA box and upstream promoter elements. The TATA box, located 25-30 base pairs upstream of the transcription initiation site, is thought to be involved in directing RNA polymerase to begin RNA synthesis. The other upstream promoter elements determine the rate at which transcription is initiated.

Enhancer elements can stimulate transcription up to 1,000 fold from linked homologous or heterologous promoters. Enhancers are active when placed downstream or upstream from the transcription initiation site. Many enhancer elements derived from viruses have a broad host range and are active in a variety of tissues. For example, the SV40 early gene enhancer is suitable for many cell types. Other enhancer/promoter combinations that are suitable for some embodiments of the invention include those derived from polyoma virus, human or murine cytomegalovirus (CMV), the long term repeat from various retroviruses such as murine leukemia virus, murine or Rous sarcoma virus and HIV. See, Enhancers and Eukaryotic Expression, Cold Spring Harbor Press, Cold Spring Harbor, N.Y. 1983, which is incorporated herein by reference.

In the construction of the expression vector, the promoter is preferably positioned approximately the same distance from the heterologous transcription start site as it is from the transcription start site in its natural setting. As is known in the art, however, some variation in this distance can be accommodated without loss of promoter function.

Polyadenylation sequences can also be added to the expression vector in order to increase the efficiency of mRNA translation. Two distinct sequence elements are required for accurate and efficient polyadenylation: GU or U rich sequences located downstream from the polyadenylation site and a highly conserved sequence of six nucleotides, AAUAAA, located 11-30 nucleotides upstream. Termination and polyadenylation signals that are suitable for some embodiments of the invention include those derived from SV40.

In addition to the elements already described, the expression vector of some embodiments of the invention may typically contain other specialized elements intended to increase the level of expression of cloned nucleic acids or to facilitate the identification of cells that carry the recombinant DNA. For example, a number of animal viruses contain DNA sequences that promote the extra chromosomal replication of the viral genome in permissive cell types. Plasmids bearing these viral replicons are replicated episomally as long as the appropriate factors are provided by genes either carried on the plasmid or with the genome of the host cell.

The vector may or may not include a eukaryotic replicon. If a eukaryotic replicon is present, then the vector is amplifiable in eukaryotic cells using the appropriate selectable marker. If the vector does not comprise a eukaryotic replicon, no episomal amplification is possible. Instead, the recombinant DNA integrates into the genome of the engineered cell, where the promoter directs expression of the desired nucleic acid.

The expression vector of some embodiments of the invention can further include additional polynucleotide sequences that allow, for example, the translation of several proteins from a single mRNA such as an internal ribosome entry site (IRES) and sequences for genomic integration of the promoter-chimeric polypeptide.

It will be appreciated that the individual elements comprised in the expression vector can be arranged in a variety of configurations. For example, enhancer elements, promoters and the like, and even the polynucleotide sequence(s) encoding the protein-of-interest can be arranged in a “head-to-tail” configuration, may be present as an inverted complement, or in a complementary configuration, as an anti-parallel strand. While such variety of configuration is more likely to occur with non-coding elements of the expression vector, alternative configurations of the coding sequence within the expression vector are also envisioned.

Other than containing the necessary elements for the transcription and translation of the inserted coding sequence, the expression construct of some embodiments of the invention can also include sequences engineered to enhance stability, production, purification, yield or toxicity of the expressed peptide.

According to specific embodiments, the expression construct include labels for imaging in cells, such as fluorescent labels.

Examples for mammalian expression vectors include, but are not limited to, pcDNA3, pcDNA3.1(+/−), pGL3, pZeoSV2(+/−), pSecTag2, pDisplay, pEF/myc/cyto, pCMV/myc/cyto, pCR3.1, pSinRep5, DH26S, DHBB, pNMT1, pNMT41, pNMT81, which are available from Invitrogen, pCI which is available from Promega, pMbac, pPbac, pBK-RSV and pBK-CMV which are available from Strategene, pTRES which is available from Clontech, and their derivatives.

Expression vectors containing regulatory elements from eukaryotic viruses such as retroviruses can be also used. SV40 vectors include pSVT7 and pMT2. Vectors derived from bovine papilloma virus include pBV-1MTHA, and vectors derived from Epstein Bar virus include pHEBO, and p205. Other exemplary vectors include pMSG, pAV009/A+, pMT010/A+, pMAMneo-5, baculovirus pDSVE, and any other vector allowing expression of proteins under the direction of the SV-40 early promoter, SV-40 later promoter, metallothionein promoter, murine mammary tumor virus promoter, Rous sarcoma virus promoter, polyhedrin promoter, or other promoters shown effective for expression in eukaryotic cells.

As described above, viruses are very specialized infectious agents that have evolved, in many cases, to elude host defense mechanisms. Typically, viruses infect and propagate in specific cell types. The targeting specificity of viral vectors utilizes its natural specificity to specifically target predetermined cell types and thereby introduce a recombinant gene into the infected cell. Thus, the type of vector used by some embodiments of the invention will depend on the cell type transformed. The ability to select suitable vectors according to the cell type transformed is well within the capabilities of the ordinary skilled artisan and as such no general description of selection consideration is provided herein.

Various methods can be used to introduce the polynucleotide or polypeptide of some embodiments of the invention into cells. Such methods are generally described in Sambrook et al., Molecular Cloning: A Laboratory Manual, Cold Springs Harbor Laboratory, New York (1989, 1992), in Ausubel et al., Current Protocols in Molecular Biology, John Wiley and Sons, Baltimore, Md. (1989), Chang et al., Somatic Gene Therapy, CRC Press, Ann Arbor, Mich. (1995), Vega et al., Gene Targeting, CRC Press, Ann Arbor Mich. (1995), Vectors: A Survey of Molecular Cloning Vectors and Their Uses, Butterworths, Boston Mass. (1988) and Gilboa et at. [Biotechniques 4 (6): 504-512, 1986] and include, for example, stable or transient transfection, lipofection, electroporation, nucleofection, microinjection, and infection with recombinant viral vectors. In addition, see U.S. Pat. Nos. 5,464,764 and 5,487,992 for positive-negative selection methods. Currently preferred in vivo nucleic acid transfer techniques include transfection with viral or non-viral constructs, such as adenovirus, lentivirus, Herpes simplex I virus, or adeno-associated virus (AAV) and lipid-based systems.

Naked DNA or RNA, cell penetrating peptide or Viral and non-viral vectors (e.g. but not limited to liposomes, nanoparticles, mammalian vectors and the like) may be utilized as delivery vehicles in delivery of the polynucleotide or polypeptide as is known in the art. According to specific embodiments of the invention, the delivery system used is biocompatible and nontoxic.

Following are exemplary embodiments suitable for enhancing penetration of the exogenous polynucleotide or polypeptide to cells.

According to one exemplary embodiment, naked DNA or RNA [e.g., naked plasmid DNA (pDNA)] is non-viral vector, which can be produced in bacteria and manipulated using standard recombinant DNA techniques. It does not induce antibody response against itself (i.e., no anti-DNA or RNA antibodies generated) and enables long-term gene expression even without chromosome integration. Naked DNA or RNA can be introduced by numerous means, for example but not limited to, intravascular and electroporation techniques [Wolff J A, Budker V, 2005, Adv. Genet. 54: 3-20], or by jet injection [Walther W, et al., 2004, Mol. Biotechnol. 28: 121-8].

According to another exemplary embodiment, mammalian vectors are used, as further described hereinabove.

According to specific embodiments, the polynucleotide is comprised in a viral vector.

Introduction of nucleic acids by viral infection offers several advantages over other methods such as lipofection and electroporation, since higher transfection efficiency can be obtained due to the infectious nature of viruses. The viral vector may be a virus with DNA based genome of a virus with RNA based genome (i.e. positive single stranded and negative single stranded RNA viruses). Examples of viral vectors include, but are not limited to, Lentivirus, Adenovirus and Retrovirus.

A viral construct such as a retroviral construct includes at least one transcriptional promoter/enhancer or locus-defining element(s), or other elements that control gene expression by other means such as alternate splicing, nuclear RNA export, or post-translational modification of messenger. Such vector constructs also include a packaging signal, long terminal repeats (LTRs) or portions thereof, and positive and negative strand primer binding sites appropriate to the virus used, unless it is already present in the viral construct. Protocols for producing recombinant retroviruses and for infecting cells in-vitro or in-vivo with such viruses can be found in, for example, Ausubel et al., [eds, Current Protocols in Molecular Biology, Greene Publishing Associates, (1989)]. Other suitable expression vectors may be an adenovirus, a lentivirus, a Herpes simplex I virus or adeno-associated virus (AAV).

Regulatory elements that limit expression to particular cell types can also be included. Such features include, for example, promoter and regulatory elements that are specific for the desired cell type.

According to specific embodiments, expressing comprises introducing into the cell the polypeptide-of-interest (e.g. the transcription factor).

Thus, according to an aspect of the present invention, there is provided a protein preparation comprising GATA3 and OCT4 transcription factors polypeptides.

According to specific embodiments, the protein preparation further comprises a c-MYC transcription factor polypeptide.

According to specific embodiments, the protein preparation further comprises at a KLF transcription factor polypeptide.

According to specific embodiments, the protein preparation further comprises a KLF4 transcription factor polypeptide.

According to specific embodiments, the protein preparation further comprises a KLF5 transcription factor polypeptide.

According to specific embodiments, the protein preparation further comprises at least one of KLF4 and KLF5 transcription factors polypeptides.

According to specific embodiments, the protein preparation further comprises KLF4 and KLF5 transcription factors polypeptides.

According to specific embodiments, two, three or all of the transcription factors are comprised in the protein preparation e.g.: GATA3+OCT4; GATA3+OCT4+c-MYC, GATA3+OCT4+KLF (e.g. GATA3+OCT4+KLF4, GATA3+OCT4+KLF5, GATA3+OCT4+KLF4+KLF5) or GATA3+OCT4+c-MYC+KLF (e.g. GATA3+OCT4+c-MYC+KLF4, GATA3+OCT4+c-MYC+KLF5, GATA3+OCT4+c-MYC+KLF4+KLF5).

According to specific embodiments, the protein preparation comprises GATA3, OCT4, c-MYC and KLF4 polypeptides.

According to specific embodiments, the protein preparation comprises GATA3, OCT4, c-MYC, KLF4 and KLF5 polypeptides.

According to specific embodiments, the protein preparation comprises each of the transcription factors in a level above a residual level (e.g. above 0.1%).

According to specific embodiments, the protein preparation comprises each of the transcription factors to a level of purity of at least 10%.

According to specific embodiments, the protein preparation comprises GATA3 and OCT4 to a level of purity of at least 20%.

According to specific embodiments, the protein preparation comprises all of the transcription factors comprised in the preparation to a level of purity of at least 20%, at least 25%, at least 30%, at least 40%, at least 50%, at least 60%, at least 70%, at least 75%, at least 80%, at least 85%, at least 90%, at least 95% or at least 99%.

According to specific embodiments, the protein preparation comprises all the transcription factors polypeptides comprised in the preparation to a level of purity of at least 90%.

Thus, according to specific embodiments, each of the polypeptides in the protein preparation is provided in a separate formulation.

According to other specific embodiments, the polypeptides in the protein preparation are provided in a co-formulation.

According to specific embodiments, the polypeptide is provided in a formulation suitable for cell penetration that enhances intracellular delivery of the polypeptide as further described hereinbelow.

Cell-Penetrating Peptides (CPPs) are short peptides (≤40 amino acids), with the ability to gain access to the interior of almost any cell. They are highly cationic and usually rich in arginine and lysine amino acids. They have the exceptional property of carrying into the cells a wide variety of covalently and noncovalently conjugated cargoes such as proteins, oligonucleotides, and even 200 nm liposomes. Therefore, according to additional exemplary embodiment CPPs can be used to transport the polynucleotide or polypeptide to the interior of cells.

TAT (transcription activator from HIV-1), pAntp (also named penetratin, Drosophila antennapedia homeodomain transcription factor) and VP22 (from Herpes Simplex virus) are examples of CPPs that can enter cells in a non-toxic and efficient manner and may be suitable for use with some embodiments of the invention. Protocols for producing CPPs-cargos conjugates and for infecting cells with such conjugates can be found, for example L Theodore et al. [The Journal of Neuroscience, (1995) 15(11): 7158-7167], Fawell S, et al. [Proc Natl Acad Sci USA, (1994) 91:664-668], and Jing Bian et al. [Circulation Research. (2007) 100: 1626-1633].

The expression level and/or activity level of the exogenous polynucleotide and/or polypeptide expressed in the cells of some embodiments of the invention can be determined using methods known in the arts, e.g. but not limited to Northern blot analysis, PCR analysis, Western blot analysis, Immunohistochemistry, and Fluorescence activated cell sorting (FACS).

“Conditions which allow generation of an iTSC from said cell” refer to the culture conditions that affect de-differentiation/re-programming of the cells and maintenance of the TSC phenotype for at least 20 passages. Non-limiting examples of such conditions may comprise culturing time, medium composition, oxygen concentration, small molecules, cytokines and expression of an exogenous transcription factor.

“Conditions which allow rejuvenation of the cell” refer to the culture conditions that affect rejuvenation of the cells without affecting their lineage and differentiation state. Non-limiting examples of such conditions may comprise culturing time, medium composition, oxygen concentration, small molecules, cytokines and expression of an exogenous transcription factor. “Conditions which allow de-differentiation of the cell” refer to the culture conditions that affect de-differentiation of the cells without affecting their lineage. These conditions may comprise culturing time, medium composition, oxygen concentration, small molecules, cytokines and expression of an exogenous transcription factor.

According to specific embodiments the conditions are such that expressing is transient.

Thus, according to specific embodiments, the iTSC, the rejuvenated cell or the de-differentiated cell does not express the exogenous transcription factor as determined by PCR, western blot and/or flow cytometry.

According to specific embodiments, the iTSC, the rejuvenated cell or the de-differentiated cell does not comprise the exogenous transcription factor as determined by PCR, western blot and/or flow cytometry.

According to specific embodiments the conditions are such that expressing is for at least 14 days, at least 15 days, at least 20 days, at least 25 days following introducing of the exogenous transcription factor into the cell.

According to specific embodiments, the conditions are such that expressing is for at least 14 days following introducing the exogenous transcription factor into the cell.

According to specific embodiments the conditions are such that expressing is for no more than 28 days, no more than 30 days, or no more than 40 days following introducing of the exogenous transcription factor into the cell.

According to specific embodiments, the conditions are such that expressing is for no more than 28 days following introducing the exogenous transcription factor into the cell.

According to specific embodiments, the conditions are such that expressing is for no more than 30 days following introducing the exogenous transcription factor into the cell.

According to specific embodiments, the conditions are such that expressing is for 14-28 days following introducing the exogenous transcription factor into the cell.

According to specific embodiments, the conditions are such that expressing is for at least 1 days, at least 3 days, at least 6 days, at least 9 days, at least 12 days, or at least 18 days following introducing the exogenous transcription factor into the cell.

According to specific embodiments the conditions are such that expressing is for no more than 30 days, no more than 25 days, no more than 20 days, or no more than 15 days following introducing of the exogenous transcription factor into the cell.

According to specific embodiments the conditions are such that expressing is for less than 14 days, following introducing of the exogenous transcription factor into the cell.

According to specific embodiments, the conditions are such that the reprogramming is performed in the absence of eggs, embryos, embryonic stem cells (ESCs) or iPSCs. Thus, any of these components are missing from the culture system.

According to specific embodiments, the conditions comprise a low oxygen concentration, e.g. 2-10% oxygen e.g. about 5% oxygen.

According to specific embodiments, the conditions comprise a culture medium comprising EGF, CHIR99021, A83-01, SB431542, Y27632 and/or VPA or TSA.

According to specific embodiments, the conditions comprise a DMEM/F12 culture medium comprising 2-mercaptoethanol, FBS, Penicillin-Streptomycin, BSA, ITS supplement, L-ascorbic acid, EGF, CHIR99021, A83-01, SB431542, VPA or TSA and Y27632, as further described hereinbelow.

According to specific embodiments, the method comprising isolating the iTSC, the rejuvenated cell or the de-differentiated cell.

Methods of isolating cells are well known in the art and include mechanical and marker based techniques. Non-limiting examples of isolating techniques include cell sorting of cells via fluorescence activated cell sorter (FACS), magnetic separation using magnetically-labeled antibodies and magnetic separation columns (e.g. MACS, Miltenyi) and manual picking under the microscope.

According to specific embodiments, cell isolation is effected by picking the iTSC colonies under the binocular/microscope followed by trypsinization and culturing in a plate containing feeder cells.

According to specific embodiments, the isolation process yields a population comprising at least about 10%, at least about 12%, at least about 14%, at least about 16%, at least about 18%, at least about 20%, at least about 22%, at least about 24%, at least about 26%, at least about 28%, at least about 30%, at least about 32%, at least about 34%, at least about 36%, at least about 38%, at least about 40%, at least about 42%, at least about 44%, at least about 46%, at least about 48%, at least about 50%, at least about 55%, at least about 60%, at least about 65%, at least about 70%, at least about 75%, at least about 80%, at least about 85%, at least about 90%, at least about 95%, at least about 96%, at least about 97%, at least about 98%, at least about 99%, e.g., 100% of the iTSCs, rejuvenated cell or de-differentiated cell of some embodiments of the invention.

According to specific embodiments, the method is effected ex-vivo or in-vitro.

As the cells (iTSCs, rejuvenated cells, de-differentiated cells) disclosed herein are generated by expressing the transcription factors disclosed herein in a cell; according to another aspect of the present invention, there is provided an isolated human cell expressing exogenous GATA3 and OCT4 transcription factors.

According to specific embodiments, the isolated cell further expresses an exogenous c-MYC transcription factor.

According to specific embodiments, the isolated cell further expresses an exogenous KLF transcription factor.

According to specific embodiments, the isolated cell further expresses an exogenous KLF4 transcription factor.

According to specific embodiments, the isolated cell further expresses an exogenous KLF5 transcription factor.

According to specific embodiments, the isolated cell further expresses at least one of exogenous KLF4 and KLF5 transcription factors.

According to specific embodiments, the isolated cell further expresses exogenous KLF4 and KLF5 transcription factors.

According to specific embodiments, the cell is comprised in a homogenous population of cells, thus, according to an aspect of the present invention, there is provided an isolated population of cells, wherein at least 80%, at least 85%, at least 90%, at least 95%, at least 97%, at least 98% of the cells are the cells disclosed herein.

According to other specific embodiments, the cell is comprised in a heterogeneous population of cells, i.e. in a population which comprises more than one cell type, e.g. in which at least 5%, at least 10%, at least 15%, at least 20%, at least 30% are the cells disclosed herein.

According to specific embodiments, the isolated cell expresses two, three or all of the transcription factors disclosed herein, e.g. GATA3+OCT4; GATA3+OCT4+c-MYC, GATA3+OCT4+KLF4 (e.g. GATA3+OCT4+KLF4, GATA3+OCT4+KLF5, GATA3+OCT4+KLF4+KLF5), GATA3+OCT4+c-MYC+KLF (e.g. GATA3+OCT4+c-MYC+KLF4, GATA3+OCT4+c-MYC+KLF5, GATA3+OCT4+c-MYC+KLF4+KLF5).

According to specific embodiments, the isolated cell expresses GATA3, OCT4, c-MYC and KLF4.

According to specific embodiments, the isolated cell expresses GATA3, OCT4, c-MYC, KLF4 and KLF5.

According to specific embodiments, the isolated cell comprises a DNA molecule encoding the transcription factors disclosed herein. Methods of evaluating the presence of an exogenous DNA molecule are known in the art and include, but are not limited to, DNA sequencing, Southern blotting, FISH and PCR.

According to specific embodiments, the isolated cell comprises a RNA molecule encoding the transcription factors disclosed herein. Methods of evaluating the presence of an exogenous RNA molecule are known in the art and include, but are not limited to, RNA sequencing, Northern blotting and PCR.

According to specific embodiments, the isolated cell comprises a protein molecule of the transcription factors disclosed herein. Methods of evaluating the presence of an exogenous protein molecule are known in the art and include, but are not limited to western blot, immunoprecipitation, immunocytochemistry and flow cytometry.

According to specific embodiments, the isolated cell is de-differentiated from a somatic cell. At times such cell may still comprise markers of origin i.e., of the source somatic cell.

According to specific embodiments, once obtained, the cells are cultured in a medium and being serially passaged.

Thus, according to an aspect of the present invention, there is provided a cell culture comprising the isolated cell of some embodiments of the invention and a culture medium.

According to an aspect of the present invention, there is provided a cell culture comprising the isolated iTSC and a culture medium.

According to an aspect of the present invention, there is provided a cell culture comprising the isolated rejuvenated cell and a culture medium.

According to an aspect of the present invention, there is provided a cell culture comprising the isolated de-differentiated cell and a culture medium.

According to specific embodiments, the culture comprises a feeder cell layer such as, but not limited to, mouse embryonic feeder (MEF) cells, human embryonic fibroblasts or adult fallopian epithelial cells and human foreskin feeder layer. Typically, feeder cell layers secrete factors needed for stem cell proliferation, while at the same time, inhibit their differentiation.

The cell culture of some embodiments can be maintained in vitro, under culturing conditions, in which the cells are being passaged for extended periods of time (e.g., for at least 20 passages, e.g., at least about 30, 40, 50, 60, 70, 80, 90, 100 passages or more), while maintaining the cell differentiation level (i.e. their TSC undifferentiated state).

It should be noted that culturing the cell (e.g. iTSC) involves replacing the culture medium with a “fresh” medium (of identical composition) every 24-72 hours, and passaging each culture dish (e.g., a plate) every once—three times a week days. Thus, when cells in the culture reach about 60-90% confluence the supernatant is discarded, the culture dishes are washed [e.g., with phosphate buffered saline (PBS)] and the cells are subjected to enzymatic dissociation from the culture dish, e.g., using trypsinization (0.25% or 0.05% Trysin+EDTA or TrypLE™ Select Enzyme Gibco), e.g., until single cells or cell clumps are separated from each other.

It should be noted that the culture conditions of some embodiments enable maintenance of the iTSC in their undifferentiated state without the need of further exogenous expression of the transcription factors.

According to specific embodiments, the method comprising assaying generation of iTSC, rejuvenation or de-differentiation.

Non-limiting examples of assays that can be used to evaluate iTSC are described in details hereinabove and below and in the Examples section which follows.

According to specific embodiments, during the culturing step cells are further monitored for their differentiation state. Cell differentiation or de-differentiation can be determined by evaluating cell morphology, or by examination of cell or tissue-specific markers, which are known to be indicative of differentiation. For example, undifferentiated human iTSC may express the TSC specific markers KRT7, GATA2, GATA3, TFAP2A, TFAP2C, TP63. In contrast, differentiated cells express other specific markers, thus for example fibroblast specific markers include THY1, ZEB1, VIM, ACTA2; cardiomyocytes specific markers include Troponin2.

Tissue/cell specific markers can be detected using immunological techniques well known in the art [Thomson J A et al., (1998). Science 282: 1145-7]. Examples include, but are not limited to, flow cytometry for membrane-bound markers and also for intracellular markers, immunohistochemistry for extracellular and intracellular markers and enzymatic immunoassay, for secreted molecular markers.

Methods useful for monitoring the expression level of specific genes are well known in the art and include RT-PCR, semi-quantitative RT-PCR, Northern blot, RNA in situ hybridization, Western blot analysis and immunohistochemistry.

Determination of undifferentiated or de-differentiation state can also be effected by evaluating the cells differentiating potential both in-vitro and in-vivo.

For example, determination of iTSC undifferentiated state can be effected by evaluating their differentiating potential both in-vitro and in-vivo by methods well known in the art such as, but not limited to, growing the cells in specified differentiation culture medium, and formation of a trophoblastic hemorrhagic lesion, localization to the extraembryonic region of the Blastocyst or localization to the placenta of the developing embryo.

In addition to monitoring a differentiation state, the cells are often also being monitored for genomic stability, transcriptome and/or methylation pattern by methods well known in the art, and compared to the corresponding species.

Non-limiting examples of assays that can be used to evaluate rejuvenation are described in details hereinabove and below.

For example, cell identity may be assessed by morphology, immunohistochemistry, transcriptome analysis (RNA-seq) etc; while rejuvenation may be evaluated, for example, by the DNA methylation clock using bisulfite sequencing, telomere length, histone marks, mitochondrial activity, gene expression and functional assays.

Function of the rejuvenated cells may also be evaluated. Non-limiting examples of functional assays that can be effected in the context of the rejuvenation aspects disclosed herein include mitochondrial activity using MitoSOX reagent and/or Seahorse XF Analyzers; DNA damage response using quantification of the basal number of γH2A.X foci and staining for the DNA damage biomarkers ATM, 53BP1, RAD51; and/or senescence by 0-gal staining. Rejuvenated mesenchymal cells may further be evaluated by a wound-healing assay using IncuCyte® S3 and migration through transwell. Rejuvenated MSCs may further be evaluated for improved immunosuppression by co-culturing the cells with peripheral blood mononuclear cells (PBMCs) and examination of their proliferation rate. With regard to CD34+ stem cells, cord blood comprises about 50% B-cells and 20% myeloid cells while adult blood comprises >50% myeloid cells and about 10-15% B-cells. Hence, one way to demonstrate cell rejuvenation of CD34+ cells is by gaining above 20% B-cells following rejuvenation, as opposed to control cells that should show about 10% of B-cells. Additionally or alternatively, presence of CD5+ cells is evaluated to explore possible generation of B1 cells, which are most restricted to youngest (fetal-liver) HSCs, in contrast to “adult” B2 cells which are CD5− cells. The functionality of the rejuvenated CD34+ cells may be further assessed in-vivo by transplantation into NSG mice. In addition, the tumorigenic potential of the rejuvenated cells may be evaluated by subcutaneous transplantation into NOD/SCID mice.

As used herein the phrase “culture medium” refers to a solid or a liquid substance used to support the growth of cell. According to specific embodiments the culture medium is a liquid medium.

According to specific embodiments, the culture medium comprises composition of components that have been shown to support culture of human TSCs, as further described herein.

According to specific embodiments the culture medium is capable of maintaining the iTSC in their differentiation state (i.e. an undifferentiated state).

According to specific embodiments, the culture medium is capable of maintaining the iTSCs in their differentiation level for at least 20 passages, e.g., at least about 30, 40, 50, 60, 70, 80, 90, 100 passages or more.

According to a specific embodiment, the culture medium is capable of maintaining the iTSCs in their differentiation level for at least 20 passages.

The culture medium used by some embodiments of the present invention can be a water-based medium which includes a combination of substances such as salts, nutrients, minerals, vitamins, amino acids, nucleic acids, proteins such as cytokines, growth factors and hormones, all of which are needed for cell proliferation and are capable of maintaining the stem cells in an undifferentiated state. For example, a culture medium can be a synthetic tissue culture medium such as RPMI (Gibco-Invitrogen Corporation products, Grand Island, NY, USA), Ko-DMEM (Gibco-Invitrogen Corporation products, Grand Island, NY, USA), DMEM/F12 (Gibco-Invitrogen Corporation products, Grand Island, NY, USA), or DMEM/F12 (Biological Industries, Biet Haemek, Israel), supplemented with the necessary additives as is further described hereinunder. Preferably, all ingredients included in the culture medium are substantially pure, with a tissue culture grade.

According to specific embodiments, the culture medium is DMEM/F12.

It will be appreciated that any of the proteinaceous factors used in the culture medium of some embodiments of the invention can be recombinantly expressed or biochemically synthesized. In addition, naturally occurring proteinaceous factors can be purified from biological samples (e.g., from human serum, cell cultures) using methods well known in the art.

According to specific embodiments, the culture medium comprises a conditioned medium. A conditioned medium is the growth medium of a monolayer cell culture (i.e., feeder cells) present following a certain culturing period. The conditioned medium includes growth factors and cytokines secreted by the monolayer cells in the culture.

According to specific embodiments, the culture medium is devoid of conditioned medium.

According to some embodiments of the invention, the culture medium is devoid of serum, e.g., devoid of any animal serum.

According to some embodiments of the invention, the culture medium is devoid of any animal contaminants, i.e., animal cells, fluid or pathogens (e.g., viruses infecting animal cells), e.g., being xeno-free.

According to some embodiments of the invention, the culture medium is devoid of human derived serum.

According to some embodiments of the invention, the culture medium further comprises serum replacement, such as but not limited to, KNOCKOUT™ Serum Replacement (Gibco-Invitrogen Corporation, Grand Island, NY USA), ALBUMAX® II (Gibco®; Life Technologies—Invitrogen, Catalogue No. 11021-029; Lipid-rich bovine serum albumin for cell culture) or a chemically defined lipid concentrate (Gibco®; Invitrogen, Life Technologies—Invitrogen, Catalogue No. 11905-031).

According to specific embodiments, the culture medium is devoid of serum replacement.

According to some embodiments of the invention, the culture medium can further include antibiotics (e.g., PEN-STREP), L-glutamine, NEAA (non-essential amino acids).

According to a specific embodiment, the medium comprises 2-mercaptoethanol, FBS, Penicillin-Streptomycin, BSA, ITS supplement, L-ascorbic acid, EGF, CH1R99021, A83-01, SB431542 and/or VPA or TSA and Y27632.

According to a specific embodiment, the medium comprises 0.1 mM 2-mercaptoethanol, 0.2% FBS, 0.5% Penicillin-Streptomycin, 0.3% BSA, 1% ITS supplement, 1.5 μg/ml L-ascorbic acid, 50 ng/ml EGF, 2 μM CHIR99021, 0.5 μM A83-01, 1 μM SB431542, 0.8 mM VPA or 10 nM TSA and 5 μM Y27632, as described in described in Okae et al. Cell Stem Cell. (2018) January 4; 22(1):50-63.

In addition to the primary cultures, the isolated cells, the iTSC, the rejuvenated cells and/or de-differentiated cells disclosed herein can be used to generate cell lines, iTSC lines, rejuvenated cell lines or de-differentiated cell lines, which are capable of unlimited expansion in culture.

Cell lines of some embodiments of the invention can be produced by immortalizing the isolated cell, iTSCs, rejuvenated cells and/or de-differentiated cells by methods known in the art, including, for example, expressing a telomerase gene in the cells (Wei, W. et al., 2003. Mol Cell Biol. 23: 2859-2870) or co-culturing the cells with NIH 3T3 hph-HOX11 retroviral producer cells (Hawley, R. G. et al., 1994. Oncogene 9: 1-12).

According to an aspect of some embodiments of the invention there is provided a method of generating differentiated cells, comprising subjecting the iTSC or de-differentiated cells of some embodiments of the invention to differentiating conditions, thereby generating the differentiated cells. Methods of differentiating iTSC into a particular cell type are known in the art and the present invention contemplates all such methods such as disclosed e.g. in Okae et al. Cell Stem Cell. 2018 Jan. 4; 22(1):50-63 and Haider et al. Stem Cell Reports. 2018 Aug. 14; 11(2):537-551, the contents of which are fully incorporated herein by reference; and include culturing the cells in a medium devoid of factors supporting the undifferentiated state e.g. when cultured in DMEM medium with 10% FBS or in a medium conducive to directed differentiation. The method may involve genetic modification of the cells and/or culturing of the cells in media comprising differentiating factors. It will be appreciated that the re-differentiating stage may result in the generation of fully differentiated cells or partially differentiated cells along a particular lineage.

According to specific embodiments of the invention, the iTSC of some embodiments of the invention can be used to isolate lineage specific cells.

As used herein, the phrase “isolating lineage specific cells” refers to the enrichment of a mixed population of cells in a culture with cells predominantly displaying at least one characteristic associated with a specific lineage phenotype. Thus, for example an iTSC can be differentiated into any of the trophoblast cell lineages. Lineage specific cells can be obtained by directly inducing the expanded, undifferentiated iTSC to culturing conditions suitable for the differentiation of specific cell lineage by methods well known in the art. It will be appreciated that the culturing conditions suitable for the differentiation and expansion of the isolated lineage specific cells include various tissue culture medium, growth factors, antibiotic, amino acids and the like and it is within the capability of one skilled in the art to determine which conditions should be applied in order to expand and differentiate particular cell types and/or cell lineages.

The invention, according to some embodiments thereof, contemplates the use of cells, tissues and organs generated from the iTSC disclosed herein using any differentiation protocol known in the art.

The isolated cells and constructs of disclosed herein may be further used for e.g. disease modeling, drug screening, and patient-specific cell-based therapy.

Thus, according to an aspect of the present invention, there is provided an isolated aggregate, organoid, placenta, developing embryo or synthetic embryo comprising the iTSC, the construct or the protein preparation disclosed herein.

According to another aspect of the present invention, there is provided a method of augmenting a placenta, a developing embryo or a synthetic embryo comprising introducing into a placenta, a developing embryo or a synthetic embryo the iTSC, the construct or the protein preparation disclosed herein.

As used herein the term “developing embryo” refers to an embryo at any stage of development and includes an embryo at a 4-cell stage, 8-cell stage, 16-cell stage embryo, early morula, late morula, early blastocyst, and/or a late blastocyst.

Methods of in-vitro or in vivo administration of cells into a placenta of a developing embryo of an animal are well known in the art, such as in Gafni O, et al. Nature. 2013 Dec. 12; 504(7479):282-6; and Manipulating the Mouse Embryo: A Laboratory Manual, Fourth Edition. By Richard Behringer; Marina Gertsenstein; Kristina Vintersten Nagy; Andras Nagy, each of which is fully incorporated herein by reference, and are also disclosed in the materials and methods of the Examples section which follows.

According to some embodiments of the invention, introducing the cells is performed in vitro or ex vivo via direct injection or aggregation with the developing host placenta or embryo.

According to another aspect of the present invention, there is provided a method of generating an aggregate or organoid comprising trophoblasts, the method comprising introducing into a scaffold or a matrix the iTSC, the construct or the protein preparation disclosed herein.

The iTCS and iTSC-derived cell preparations and the chimeric placentas may be used to prepare model systems for disorders associated with development and/or activity of trophoblasts, to screen for genes expressed in or essential for trophoblast differentiation and/or activity, to screen for agents or conditions (such as culture conditions or manipulation) that effect trophoblast differentiation and/or activity, to produce trophoblast specific growth factors and hormones and as a cell therapy for disorders associated with development and/or activity of trophoblasts.

Consequently, the cell preparations and the chimeric placentas may be used to screen for potential agents that modulate trophoblast development or activity e.g. invasion or proliferation.

Thus, according to an aspect of the present invention, there is provided a method of identifying an agent capable of modulating trophoblast development and/or activity, the method comprising:
(i) contacting the isolated iTSC, population of cells comprising iTSC, aggregate, organoid or placenta disclosed herein with a candidate agent; and
(ii) comparing development and/or activity of the isolated iTSC, population of cells, aggregate, organoid or placenta following said contacting with said agent to development and/or activity of said isolated iTSC, population of cells, aggregate, organoid or placenta without said agent,
wherein an effect of said agent on said development and/or activity of said isolated iTSC, population of cells, aggregate, organoid or placenta above a predetermined level relative to said development and/or activity of said isolated iTSC, population of cells, aggregate, organoid or placenta without said agent is indicative that said drug modulates trophoblast development and/or activity.

As used herein, the term “modulating” refers to altering trophoblast development and/or activity either by inhibiting or by promoting.

According to specific embodiments, modulating is inhibiting development and/or activity.

According to specific embodiments, modulating is promoting development and/or activity.

For the same culture conditions, the effect of the candidate agent on trophoblast development and/or activity is generally expressed in comparison to the development and/or activity in a cell of the same species but not contacted with the candidate agent or contacted with a vehicle control, also referred to as control.

As used herein the phrase “an effect above a predetermined threshold” refers to a change in trophoblast development and/or activity following contacting with the compound which is higher than a predetermined threshold such as a about 10%, e.g., higher than about 20%, e.g., higher than about 30%, e.g., higher than about 40%, e.g., higher than about 50%, e.g., higher than about 60%, higher than about 70%, higher than about 80%, higher than about 90%, higher than about 2 times, higher than about three times, higher than about four time, higher than about five times, higher than about six times, higher than about seven times, higher than about eight times, higher than about nine times, higher than about 20 times, higher than about 50 times, higher than about 100 times, higher than about 200 times, higher than about 350, higher than about 500 times, higher than about 1000 times, or more relative to the level of expression prior to contacting with the compound.

According to specific embodiments, the candidate agent may be any compound including, but not limited to a chemical, a small molecule, a polypeptide and a polynucleotide.

The cell preparations, aggregates, organoids and placentas can also be used to identify genes and substances that are important for the trophoblast development and/or activity. The isolated iTSC can also be modified by introducing mutations into genes in the cells or by introducing transgenes into the cells.

According to specific embodiments, the selected agents may be further used to treat various conditions requiring regulation of trophoblast development or activity such as the conditions described below.

Recurrent miscarriage and fetal growth restriction (FGR) are associated with placental dysfunction and contribute to handicaps and in severe cases death. Cellular transplantation of intact and healthy TSCs holds great promise in the clinic as the transplanted cells might be able to rescue some of these fetuses by supporting the undeveloped/damaged placenta.

Thus, according to another aspect of the present invention, there is provided a method of treating and/or preventing a disorder associated with development and/or activity of trophoblasts in a subject in need thereof, the method comprising administering to the subject a therapeutically effective amount of the iTSC, the construct or the protein preparation disclosed herein, thereby treating and/or preventing the disorder associated with development and/or activity of trophoblasts in the subject.

According to an additional or an alternative aspect of the present invention, there is provided the iTSC, the construct or the protein preparation disclosed herein, for use in treating and/or preventing a disorder associated with development and/or activity of trophoblasts in a subject in need thereof.

This aspect of the present invention contemplates treating a disorder associated with development and/or activity of trophoblasts. Dysfunctional trophoblasts may affect on the one had the mother and the other hand the fetus. Hence contemplated are both conditions. Non-limiting examples of such disorders include recurrent miscarriage, Preeclampsia, Fetal Growth Restriction (FGR), hydatiform mole and choriocarcinoma.

The terms “treating” or “treatment” refers to inhibiting or arresting the development of a pathology (e.g. recurrent miscarriage) and/or causing the reduction, remission, or regression of a pathology. Those of skill in the art will understand that various methodologies and assays can be used to assess the development of a pathology, and similarly, various methodologies and assays may be used to assess the reduction, remission or regression of a pathology.

As used herein, the term “preventing” refers to keeping a disease (or pathology) from occurring in a subject who may be at risk for the disease, but has not yet been diagnosed as having the disease.

As used herein the phrase “subject in need thereof” refers to a mammalian subject (e.g., human being) who is diagnosed with the pathology. In a specific embodiment, this term encompasses individuals who are at risk to develop the pathology. Veterinary uses are also contemplated. The subject may be of any gender or at any age including neonatal, infant, juvenile, adolescent, adult and elderly adult. According to specific embodiments, the subject is a female.

According to specific embodiments, the subject is at least 20 years old.

According to specific embodiments, the subject is at least 40 years old.

According to specific embodiments, the subject is at least 50 years old.

According to specific embodiments, the subject is at least 60 years old.

According to specific embodiments, the subject is at least 70 years old.

As trophoblasts produce several secreted growth factors and hormones, according to another aspect of the present invention, there is provided a method of obtaining a compound produced by a trophoblast, the method comprising culturing the isolated iTSC, the population of cells comprising iTSC or the iTCS cell culture disclosed herein and isolating from the culture medium a compound secreted by the cells, thereby obtaining the compound produced by the trophoblast.

According to specific embodiments, the compound is a growth factor or a hormone, such as but not limited to human Chorionic Gonadotropin (hCG).

According to an additional or an alternative aspect of the present invention, there is provided a method of treating and/or preventing a disease associated with aging in a subject in need thereof, the method comprising administering to the subject a therapeutically effective amount of the rejuvenated cell, the de-differentiated cell, the construct or the protein preparation disclosed herein, thereby treating and/or preventing the disease in the subject.

According to an additional or an alternative aspect of the present invention, there is provided the rejuvenated cell, the de-differentiated cell, the construct or the protein preparation disclosed herein for use in treating and/or preventing a disease associated with aging in a subject in need thereof.

This aspect of the present invention contemplates treating a disease associated with aging. Non-limiting examples of such diseases include glaucoma, cataract, high myopia, retinitis pigmentosa, cone dystrophy, cone-rod dystrophy, Usher syndrome, Stargardt disease, Barder-Biedell syndrome, Best disease, inherited maculopathy, Myelodysplastic syndromes (MDS), cancer, graft rejection, graft versus host disease (GVHD), infectious disease, cytokine storm, radiation damage, neurodegenerative disease and wound.

According to specific embodiments, the disease associated with aging results from increased senescence.

According to specific embodiments, the disease is a vision related disease.

According to specific embodiments, the disease is selected from the group consisting of glaucoma, cataract, high myopia, retinitis pigmentosa, cone dystrophy, cone-rod dystrophy, Usher syndrome, Stargardt disease, Barder-Biedell syndrome, Best disease and inherited maculopathy.

According to specific embodiments, the disease is selected from the group consisting of Myelodysplastic syndromes (MDS), cancer, graft rejection, graft versus host disease (GVHD), infectious disease, cytokine storm, radiation damage, neurodegenerative disease and wound.

Since the constructs and protein preparations disclosed herein induce rejuvenation and de-differentiation of cells, the present inventors contemplate that another use thereof is in cosmetic compositions as anti-aging agents e.g. for rejuvenating the skin.

Thus, according to an additional or an alternative aspect of the present invention, there is provided a method of performing a cosmetic care in a subject in need thereof, the method comprising applying to the skin of the subject a therapeutically effective amount of the construct or the protein preparation disclosed herein, thereby performing the cosmetic care.

The cells, constructs and protein preparations disclosed herein, may be transplanted to a subject per se, or may be formulated in compositions intended for a particular use. Similarly, the constructs and protein preparations disclosed herein may be administered to a subject per se, or in formulated in a composition intended for a particular use.

For treatment of diseases the cells, constructs or protein preparations disclosed herein may be formulated in a pharmaceutical composition where they are mixed with suitable carriers or excipients.

As used herein a “pharmaceutical composition” refers to a preparation of one or more of the active ingredients described herein with other chemical components such as physiologically suitable carriers and excipients. The purpose of a pharmaceutical composition is to facilitate administration of a compound to an organism.

Herein the term “active ingredient” refers to the cells (e.g. iTSC, rejuvenated cells, de-differentiated cells), the construct or the protein preparation disclosed herein accountable for the biological effect.

Hereinafter, the phrases “physiologically acceptable carrier” and “pharmaceutically acceptable carrier” which may be interchangeably used refer to a carrier or a diluent that does not cause significant irritation to an organism and does not abrogate the biological activity and properties of the administered compound. Herein the term “excipient” refers to an inert substance added to a pharmaceutical composition to further facilitate administration of an active ingredient. Examples, without limitation, of excipients include calcium carbonate, calcium phosphate, various sugars and types of starch, cellulose derivatives, gelatin, vegetable oils and polyethylene glycols.

Techniques for formulation and administration of drugs may be found in “Remington's Pharmaceutical Sciences,” Mack Publishing Co., Easton, PA, latest edition, which is incorporated herein by reference.

Pharmaceutical compositions of some embodiments of the present invention may be manufactured by processes well known in the art, e.g., by means of conventional mixing, dissolving, granulating, dragee-making, levigating, emulsifying, encapsulating, entrapping or lyophilizing processes.

Pharmaceutical compositions for use in accordance with some embodiments of the present invention thus may be formulated in conventional manner using one or more physiologically acceptable carriers comprising excipients and auxiliaries, which facilitate processing of the active ingredients into preparations, which can be used pharmaceutically. Proper formulation is dependent upon the route of administration chosen.

For injection, the active ingredients of the pharmaceutical composition may be formulated in aqueous solutions, preferably in physiologically compatible buffers such as Hank's solution, Ringer's solution, or physiological salt buffer.

Suitable routes of administration may, for example, include oral, rectal, transmucosal, especially transnasal, intestinal or parenteral delivery, including intramuscular, subcutaneous and intramedullary injections as well as intrathecal, direct intraventricular, intracardiac, e.g., into the right or left ventricular cavity, into the common coronary artery, intravenous, intraperitoneal, intranasal, or intraocular injections.

According to specific embodiments, the pharmaceutical composition is administered in a local rather than systemic manner, for example, via injection of the pharmaceutical composition directly into a tissue region of a patient.

The pharmaceutical composition described herein may be formulated for parenteral administration, e.g., by bolus injection or continuous infusion. Formulations for injection may be presented in unit dosage form, e.g., in ampoules or in multidose containers with optionally, an added preservative. The compositions may be suspensions, solutions or emulsions in oily or aqueous vehicles, and may contain formulatory agents such as suspending, stabilizing and/or dispersing agents.

Pharmaceutical compositions for parenteral administration include aqueous solutions of the active preparation in water-soluble form. Additionally, suspensions of the active ingredients may be prepared as appropriate oily or water based injection suspensions. Suitable lipophilic solvents or vehicles include fatty oils such as sesame oil, or synthetic fatty acids esters such as ethyl oleate, triglycerides or liposomes. Aqueous injection suspensions may contain substances, which increase the viscosity of the suspension, such as sodium carboxymethyl cellulose, sorbitol or dextran. Optionally, the suspension may also contain suitable stabilizers or agents which increase the solubility of the active ingredients to allow for the preparation of highly concentrated solutions.

Pharmaceutical compositions suitable for use in context of the present invention include compositions wherein the active ingredients are contained in an amount effective to achieve the intended purpose. More specifically, a therapeutically effective amount means an amount of active ingredients (e.g. iTSCs) effective to prevent, alleviate or ameliorate symptoms of a disorder (e.g., recurrent miscarriage) or prolong the survival of the subject being treated.

Determination of a therapeutically effective amount is well within the capability of those skilled in the art, especially in light of the detailed disclosure provided herein.

For any preparation used in the methods of the invention, the therapeutically effective amount or dose can be estimated from animal models to achieve a desired concentration or titer. Such information can be used to more accurately determine useful doses in humans.

Toxicity and therapeutic efficacy of the active ingredients described herein can be determined by standard pharmaceutical procedures in experimental animals. The data obtained from these animal studies can be used in formulating a range of dosage for use in human. The dosage may vary depending upon the dosage form employed and the route of administration utilized. The exact formulation, route of administration and dosage can be chosen by the individual physician in view of the patient's condition. (See e.g., Fingl, et al., 1975, in “The Pharmacological Basis of Therapeutics”, Ch. 1 p. 1).

Dosage amount and interval may be adjusted individually to provide levels of the active ingredient are sufficient to induce or suppress the biological effect (minimal effective concentration, MEC). The MEC will vary for each preparation, but can be estimated from in vitro data. Dosages necessary to achieve the MEC will depend on individual characteristics and route of administration. Detection assays can be used to determine plasma concentrations of C peptide and/or insulin.

The amount of a composition to be administered will, of course, be dependent on the subject being treated, the severity of the affliction, the manner of administration, the judgment of the prescribing physician, etc.

Compositions of the present invention may, if desired, be presented in a pack or dispenser device, such as an FDA approved kit, which may contain one or more unit dosage forms containing the active ingredient. The pack may, for example, comprise metal or plastic foil, such as a blister pack. The pack or dispenser advice may be a syringe. The syringe may be prepacked with the cells. The pack or dispenser device may be accompanied by instructions for administration. The pack or dispenser may also be accommodated by a notice associated with the container in a form prescribed by a governmental agency regulating the manufacture, use or sale of pharmaceuticals, which notice is reflective of approval by the agency of the form of the compositions or human or veterinary administration. Such notice, for example, may be of labeling approved by the U.S. Food and Drug Administration for prescription drugs or of an approved product insert. Compositions comprising a preparation of the invention formulated in a compatible pharmaceutical carrier may also be prepared, placed in an appropriate container, and labeled for treatment of an indicated condition, as if further detailed above.

For cosmetics, the constructs or protein preparations disclosed herein may be formulated in a cosmetic composition where they are mixed with suitable carriers or excipients, e.g. a dermatologically acceptable suitable for external topical application.

According to specific embodiments, the cosmetic composition is formulated as a cream, a face mask, a scrub, a soap, a wash or a gel.

The cosmetic composition according to some embodiments of the present invention may further comprise at least one pharmaceutical adjuvant known to the person skilled in the art, selected from thickeners, preservatives, fragrances, colorants, chemical or mineral filters, moisturizing agents, thermal spring water, etc.

The composition may comprise at least one agent selected from a sebum-regulating agent, an antibacterial agent, an antifungal agent, a keratolytic agent, a keratoregulating agent, an astringent, an anti-inflammatory/anti-irritant, an antioxidant/free-radical scavenger, a cicatrizing agent, an anti-aging agent and/or a moisturizing agent.

The term “sebum-regulating agent” refers, for example, to 5-α-reductase inhibitors, notably the active agent 5-α-Avocuta® sold by Laboratoires Expanscience Zinc and gluconate salts thereof, salicylate and pyroglutamic acid, also have sebum-suppressing activity. Mention may also be made of spironolactone, an anti-androgen and aldosterone antagonist, which significantly reduces the sebum secretion rate after 12 weeks of application. Other extracted molecules, for example from seeds of the pumpkin Cucurbita pepo, and squash seed oil, as well as palm cabbage, limit sebum production by inhibiting 5-α-reductase transcription and activity. Other sebum-regulating agents of lipid origin that act on sebum quality, such as linoleic acid, are of interest.

The terms “anti-bacterial agent” and “antifungal agent” refer to molecules that limit the growth of or destroy pathogenic microorganisms such as certain bacteria like P. acnes or certain fungi (Malassezia furfur). The most traditional are preservatives generally used in cosmetics or nutraceuticals, molecules with anti-bacterial activity (pseudo-preservatives) such as caprylic derivatives (capryloyl glycine, glyceryl caprylate, etc.), such as hexanediol and sodium levulinate, zinc and copper derivatives (gluconate and PCA), phytosphingosine and derivatives thereof, benzoyl peroxide, piroctone olamine, zinc pyrithione, selenium sulfide, econazole, ketoconazole, or local antibiotics such as erythromycin and clindamycin, etc.

The terms “keratoregulating agent” and “keratolytic agent” refer to an agent that regulates or helps the elimination of dead cells of the stratum corneum of the epidermis. The most commonly used keratoregulating/keratolytic agents include: alpha-hydroxy acids (AHAs) of fruits (citric acid, glycolic acid, malic acid, lactic acid, etc.), AHA esters, combinations of AHAs with other molecules such as the combination of malic acid and almond proteins (Keratolite®) the combination of glycolic acid or lactic acid with arginine or the combination of hydroxy acid with lipid molecules such as LHA® (lipo-hydroxy acid), amphoteric hydroxy acid complexes (AHCare), willow bark (Salix alba bark extract), azelaic acid and salts and esters thereof, salicylic acid and derivatives thereof such as capryloyl salicylic acid or in combination with other molecules such as the combination of salicylic acid and polysaccharide (beta-hydroxy acid, or BHA), tazarotene, adapalene, as well as molecules of the retinoid family such as tretinoin, retinaldehyde, isotretinoin and retinol.

The term “astringent” refers to an agent that helps constrict pores, the most commonly used being polyphenols, zinc derivatives and witch hazel.

The term “anti-inflammatory/anti-irritant” refers to an agent that limits the inflammatory reaction led by cytokines or arachidonic acid metabolism mediators and has soothing and anti-irritating properties. The most traditional are glycyrrhetinic acid (licorice derivative) and salts and esters thereof, alpha-bisabolol, Ginkgo biloba, Calendula, lipoic acid, beta-carotene, vitamin B3 (niacinamide, nicotinamide), vitamin E, vitamin C, vitamin B12, flavonoids (green tea, quercetin, etc.), lycopene or lutein, avocado sugars, avocado oleodistillate, arabinogalactan, lupin peptides, lupin total extract, quinoa peptide extract, Cycloceramide® (oxazoline derivative), anti-glycation agents such as carnosine, N-acetyl-cysteine, isoflavones such as, for example, genistein/genistin, daidzein/daidzin, spring water or thermal spring water (eau d'Avene, eau de la Roche Posay, eau de Saint Gervais, eau d'Uriage, eau de Gamarde), goji extracts (Lycium barbarum), plant amino acid peptides or complexes, topical dapsone, or anti-inflammatory drugs.

The term “antioxidant” refers to a molecule that decreases or prevents the oxidation of other chemical substances. The antioxidants/free-radical scavengers that may be used in combination are advantageously selected from the group comprised of thiols and phenols, licorice derivatives such as glycyrrhetinic acid and salts and esters thereof, alpha-bisabolol, Ginkgo biloba extract, Calendula extract, Cycloceramide® (oxazoline derivative), avocado peptides, trace elements such as copper, zinc and selenium, lipoic acid, vitamin B12, vitamin B3 (niacinamide, nicotinamide), vitamin C, vitamin E, coenzyme Q10, krill, glutathione, butylated hydroxytoluene (BHT), butylated hydroxyanisole (BHA), lycopene or lutein, beta-carotene, the family of polyphenols such as tannins, phenolic acids, anthocyanins, flavonoids such as, for example, extracts of green tea, of red berries, of cocoa, of grapes, of Passiflora incarnata or of Citrus, or isoflavones such as, for example, genistein/genistin and daidzein/daidzin. The group of antioxidants further includes anti-glycation agents such as carnosine or certain peptides, N-acetyl-cysteine, as well as antioxidant or free-radical scavenging enzymes such as superoxide dismutase (SOD), catalase, glutathione peroxidase, thioredoxin reductase and agonists thereof.

The agents that cicatrize/repair the barrier function which may be used in combination are advantageously vitamin A, panthenol (vitamin B5), Avocadofurane®, avocado sugars, lupeol, maca peptide extract, quinoa peptide extract, arabinogalactan, zinc oxide, magnesium, silicon, madecassic or asiatic acid, dextran sulfate, coenzyme Q10, glucosamine and derivatives thereof, chondroitin sulfate and on the whole glycosaminoglycans (GAGs), dextran sulfate, ceramides, cholesterol, squalane, phospholipids, fermented or unfermented soya peptides, plant peptides, marine, plant or biotechnological polysaccharides such as algae extracts or fern extracts, trace elements, extracts of tannin-rich plants such as tannins derived from gallic acid called gallic or hydrolysable tannins, initially found in oak gall, and catechin tannins resulting from the polymerization of flavan units whose model is provided by the catechu (Acacia catechu). The trace elements that may be used are advantageously selected from the group comprised of copper, magnesium, manganese, chromium, selenium, silicon, zinc and mixtures thereof.

Anti-aging agents that can act in combination with the constructs and protein preparations disclosed herein are antioxidants and in particular vitamin C, vitamin A, retinol, retinal, hyaluronic acid of any molecular weight, Avocadofurane® lupin peptides and maca peptide extract.

The most commonly used moisturizers/emollients are glycerin or derivatives thereof, urea, pyrrolidone carboxylic acid and derivatives thereof, hyaluronic acid of any molecular weight, glycosaminoglycans and any other polysaccharides of marine, plant or biotechnological origin such as, for example, xanthan gum, Fucogel®, certain fatty acids such as lauric acid, myristic acid, monounsaturated and polyunsaturated omega-3, -6, -7 and -9 fatty acids (linoleic acid, palmitoleic acid, etc.), sunflower oleodistillate, avocado peptides and cupuacu butter.

As used herein the term “about” refers to ±10%.

The terms “comprises”, “comprising”, “includes”, “including”, “having” and their conjugates mean “including but not limited to”.

The term “consisting of” means “including and limited to”.

The term “consisting essentially of” means that the composition, method or structure may include additional ingredients, steps and/or parts, but only if the additional ingredients, steps and/or parts do not materially alter the basic and novel characteristics of the claimed composition, method or structure.

As used herein, the singular form “a”, “an” and “the” include plural references unless the context clearly dictates otherwise. For example, the term “a compound” or “at least one compound” may include a plurality of compounds, including mixtures thereof.

Throughout this application, various embodiments of this invention may be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals therebetween.

As used herein the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

When reference is made to particular sequence listings, such reference is to be understood to also encompass sequences that substantially correspond to its complementary sequence as including minor sequence variations, resulting from, e.g., sequencing errors, cloning errors, or other alterations resulting in base substitution, base deletion or base addition, provided that the frequency of such variations is less than 1 in 50 nucleotides, alternatively, less than 1 in 100 nucleotides, alternatively, less than 1 in 200 nucleotides, alternatively, less than 1 in 500 nucleotides, alternatively, less than 1 in 1000 nucleotides, alternatively, less than 1 in 5,000 nucleotides, alternatively, less than 1 in 10,000 nucleotides.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination or as suitable in any other described embodiment of the invention. Certain features described in the context of various embodiments are not to be considered essential features of those embodiments, unless the embodiment is inoperative without those elements.

Various embodiments and aspects of the present invention as delineated hereinabove and as claimed in the claims section below find experimental support in the following examples.

### EXAMPLES

Reference is now made to the following examples, which together with the above descriptions illustrate some embodiments of the invention in a non limiting fashion.

Generally, the nomenclature used herein and the laboratory procedures utilized in the present invention include molecular, biochemical, microbiological and recombinant DNA techniques. Such techniques are thoroughly explained in the literature. See, for example, “Molecular Cloning: A laboratory Manual” Sambrook et al., (1989); “Current Protocols in Molecular Biology” Volumes I-III Ausubel, R. M., ed. (1994); Ausubel et al., “Current Protocols in Molecular Biology”, John Wiley and Sons, Baltimore, Maryland (1989); Perbal, “A Practical Guide to Molecular Cloning”, John Wiley & Sons, New York (1988); Watson et al., “Recombinant DNA”, Scientific American Books, New York; Birren et al. (eds) “Genome Analysis: A Laboratory Manual Series”, Vols. 1-4, Cold Spring Harbor Laboratory Press, New York (1998); methodologies as set forth in U.S. Pat. Nos. 4,666,828; 4,683,202; 4,801,531; 5,192,659 and 5,272,057; “Cell Biology: A Laboratory Handbook”, Volumes I-III Cellis, J. E., ed. (1994); “Culture of Animal Cells—A Manual of Basic Technique” by Freshney, Wiley-Liss, N. Y. (1994), Third Edition; “Current Protocols in Immunology” Volumes I-III Coligan J. E., ed. (1994); Stites et al. (eds), “Basic and Clinical Immunology” (8th Edition), Appleton & Lange, Norwalk, C T (1994); Mishell and Shiigi (eds), “Selected Methods in Cellular Immunology”, W. H. Freeman and Co., New York (1980); available immunoassays are extensively described in the patent and scientific literature, see, for example, U.S. Pat. Nos. 3,791,932; 3,839,153; 3,850,752; 3,850,578; 3,853,987; 3,867,517; 3,879,262; 3,901,654; 3,935,074; 3,984,533; 3,996,345; 4,034,074; 4,098,876; 4,879,219; 5,011,771 and 5,281,521; “Oligonucleotide Synthesis” Gait, M. J., ed. (1984); “Nucleic Acid Hybridization” Hames, B. D., and Higgins S. J., eds. (1985); “Transcription and Translation” Hames, B. D., and Higgins S. J., eds. (1984); “Animal Cell Culture” Freshney, R. I., ed. (1986); “Immobilized Cells and Enzymes” IRL Press, (1986); “A Practical Guide to Molecular Cloning” Perbal, B., (1984) and “Methods in Enzymology” Vol. 1-317, Academic Press; “PCR Protocols: A Guide To Methods And Applications”, Academic Press, San Diego, CA (1990); Marshak et al., “Strategies for Protein Purification and Characterization—A Laboratory Course Manual” CSHL Press (1996); all of which are incorporated by reference as if fully set forth herein. Other general references are provided throughout this document. The procedures therein are believed to be well known in the art and are provided for the convenience of the reader. All the information contained therein is incorporated herein by reference.

### Materials and Methods

Derivation of human trophoblast stem cells from human blastocysts—In order to generate human blastocyst-derived TSC (hbdTSC) control lines, human blastocysts were plated on Mitomycin C-treated MEF feeder and cultured in human TSC medium as described in Okae et al. [Cell stem cell (2018) 22, 50-63 e56]. Following blastocyst outgrowth, the cells were trypsinized and transferred into new Mitomycin C-treated mouse embryonic fibroblast (MEF) feeder plate. The cells were passaged several times, until stable proliferative hbdTSCs emerged.

Molecular Cloning and hiTSC and hiPSC reprogramming—Dox-inducible factors were generated by cloning the open reading frame of each factor, obtained by reverse transcription with specific primers (see primers list in Table 1 hereinbelow), into the pMINI vector (NEB) and then restricted with EcoRI or MfeI and inserted into the FUW-TetO expression vector. A lentiviral vector dox-dependent system was utilized for the transient expression of transcription factors. KLF5 coding sequence was synthesized by TWIST and subcloned into FUW-TetO with EcoRI. For infection, replication-incompetent lentiviruses containing the various reprogramming factors and ratios (GOKM 2:3:3:2 or 1:1:1:0.3, GOK 4 K 5 M (1:1:1:1:0.3) for hiTSC reprogramming and OKSM STEMCCA cassette for hiPSC reprogramming) were packaged with a lentiviral packaging mix (7.5 μg psPAX2 and 2.5 μg pDGM.2) in 293T cells and collected 48, 60, 72 and 84 hours following transfection. The supernatants were filtered through a 0.45 μm filter, supplemented with 8 μg/ml of polybrene, and then used to infect Human foreskin fibroblasts (HFFs). Twelve hours following the fourth infection, medium was replaced with fresh DMEM containing 10% FBS. For hiTSC reprogramming, six hours later, 2 μg/ml doxycycline was added to the medium. For hiTSC reprogramming, the basic reprogramming medium (BRM) consisting of DMEM supplemented with 10% FBS, was changed every other day for 14 days, followed by 7 days in medium comprised of 50% BRM and 50% hTSC medium as described in Okae et al., 2018, followed by 7 days in hTSC medium as described in Okae et al., 2018, after which dox was removed. 7-10 days following dox removal, plates were screened for primary hiTSC colonies. Each colony was isolated, trypsinized with TrypLE (Gibco) and plated in a separate well of a 6-wells plate on feeder cells. The cells were passaged several times until stable proliferative hiTSC colonies emerged.

Quantitative PCR (qPCR) for mRNA expression and analysis of genomic integration of transgenes—For analysis of mRNA expression using qPCR, total RNA was isolated using the Macherey-Nagel kit (Ornat). 500-2000 ng of total RNA was reverse transcribed using iScript cDNA Synthesis kit (Bio-Rad). Quantitative PCR analysis was performed in duplicates using 1/100 of the reverse transcription reaction in a StepOnePlus (Applied Biosystems) with SYBR green Fast qPCR Mix (Applied Biosystems). Specific primers were designed for the different genes (see Table 1 hereinbelow). All quantitative real-time PCR experiments were normalized to the expression of GAPDH and presented as a mean±standard deviation of two duplicate runs.

For analysis of transgenes integration into the genomic DNA using qPCR, genomic DNA was isolated by incubating trypsinized cell pellets in lysis buffer containing 100 mM Tris pH8.0, 5 mM EDTA, 0.2% SDS and 200 mM NaCl overnight with 400 μg/ml proteinase K (Axxora) at 37° C. for one hour followed by incubation at 55° C. for one hour. Following, genomic DNA was precipitated with iso-propanol, washed with 70% ethanol and resuspended in ultra-pure water (BI). Forward primers for the end of the last exon of cloned genes were used in conjunction with reverse primers for the FUW vector at the region immediately downstream of the cloned gene (see Table 1 hereinbelow). Results were normalized to an intronic region of the GAPDH gene and presented as a mean±standard deviation of two duplicate runs.

Immunostaining of PFA-fixated cells and flow cytometry—Cells were fixed in 4% paraformaldehyde (in PBS) for 20 minutes, rinsed 3 times with PBS and blocked for 1 hour with PBS containing 0.1% triton X-100 and 5% FBS. The cells were incubated overnight with primary antibodies (1:200) in 4° C. The antibodies used were: anti-KRT7 (Abcam, ab215855), anti-GATA3 (Abcam, ab106625), anti-GATA2 (Abcam, ab173817), anti-TFAP2C (Santa Cruz Biotechnologies, sc-8977), anti-KRT18 (Santa Cruz Biotechnologies, sc-51582), anti-E-cadherin (Santa Cruz Biotechnologies, sc-7870), anti-Vimentin (Cell Signaling Technology, #5741), anti-EpCAM (Abcam, ab71916), anti-SDC1 (Abcam, ab128936), anti-CSH1 (Abcam, ab15554), anti-HLA-G (Abcam, ab52455) diluted in in PBS containing 0.1% triton X-100 and 1% FBS. The next day, the cells were washed 3 times and incubated for 1 hour with relevant (Alexa) secondary antibody in PBS containing 0.1% triton X-100 and 1% FBS (1:500 dilution). DAPI was added 10 minutes prior to the end of incubation. Negative control included incubation with secondary antibody without primary.

For flow cytometry analysis of HLA class I expression, cells were trypsinized and blocked for ten minutes in incubation buffer containing 0.5% bovine serum albumin (BSA) (Sigma Aldrich) in PBS. Following, cells were centrifuged and resuspended in incubation buffer with anti-HLA class I (Abcam, ab22432) (1:100) for 1 hour. Cells were then washed with incubation buffer and incubated for 30 minutes with relevant (Alexa) secondary antibody, after which cells were washed, resuspended in incubation buffer and analyzed by FACS (Beckman Coulter). Results were analyzed using Kaluza Software. Each sample was also incubated with a secondary antibody only as a negative control.

hCG detection using commercial pregnancy tests—A commercially available rapid pregnancy test (“Uni Test”, Core Technologies) was used for the detection of hCG present in the medium of cells. Each line of cells was seeded in a 6-wells plate well with 2 ml of appropriate medium until reaching a confluence of 60-80% (40-50% for iPSCs), and 0.5 ml was collected 24 hours later. The HFFs, which were not infected with GOKM, were seeded in a 15 cm plate with 25 ml of medium. From these, 0.5 ml was collected 72 hours later at a confluence of 70%.

RNA and RRBS libraries and sequencing—For RNAseq, total RNA was isolated using the Qiagen RNeasy kit. mRNA libraries were prepared using the SENSE mRNA-seq library prep kit V2 (Lexogen), and pooled libraries were sequenced on an Illumina NextSeq 500 platform to generate 75-bp single-end reads. For RRBS, DNA was isolated from samples and incubated in lysis buffer (25 mM Tris-HCl at pH 8, 2 mM EDTA, 0.2% SDS, 200 mM NaCl) supplemented with 300 μg/mL proteinase K (Roche) followed by phenol:chloroform extraction and ethanol precipitation. hiTSC colonies and hbdTSC colonies were passaged twice on matrigel in order to eliminate the presence of MEF feeder cells. RRBS libraries were prepared as described in Boyle et al. Genome Biol. 2012 Oct. 3; 13(10):R92. Samples were run on HiSeq 2500 (Illumina).

RNA-seq and RRBS analysis—For analysis of RNA-seq results, Raw reads (fastq files) were quality-trimmed using in-house Perl scripts, and adapters removed with cutadapt (version 1.12). The processed fastq files were mapped to the human transcriptome and genome using TopHat (v2.1.1). The genome version was GRCh38, with annotations from Ensembl release 89. Quantification was done using htseq-count (version 0.6.1). Genes with a sum of counts less than over all samples were filtered out, retaining 25596 genes. Normalization was done with the DESeq2 package (version 1.16.1).

For analysis of RRBS results, BSMAP V 2.9 was used to align the paired-end reads to the human genome (hg19), and the adapters and low-quality sequences were trimmed using Trim Galore. The methylation ratio of CpGs with sequencing depth of at least 10 reads were computed based on 100 bp tiles.

Differentiation of hiTSCs and staining with PI—Two hbdTSC and two hiTSC lines were seeded on Matrigel-coated 6-wells plates in hTSC medium and allowed to reach 70% confluency. Following, medium was switched to a basic differentiation medium of DMEM supplemented with 10% FBS supplemented with 1% L-glutamine solution (BI) and antibiotics (BDM). Cells were harvested at day 0 and every day for five days from the six identical wells for gene expression analysis.

For directed differentiation into ST, approximately 4×10⁵ cells were seeded on Matrigel coated 6-wells plates at a concentration of 1:30 in ambient oxygen conditions in a medium consisting of DMEM/F12 supplemented with 0.1 mM 2-mercaptoethanol, 0.5% Penicillin-Streptomycin, 0.3% BSA, 1% ITS supplement, 2.5 μM Y27632, 2 μM forskolin, and 4% KSR, as described by Okae et al. Cell Stem Cell. 2018 Jan. 4; 22(1):50-63. Cells were collected at day 2 and 6 for analysis of mRNA expression using qPCR as described above. Cells were also seeded on 12-wells plates at a density of approximately 10⁵ cells per plate, cultured similarly and fixated in 4% PFA for immunostaining as described above.

For directed differentiation into EVT, approximately 4×10⁵ cells were seeded on Matrigel coated 6-well plates at a concentration of 1:100 in 5% oxygen in a DMEM/F12 medium supplemented with 0.1 mM 2-mercaptoethanol, 0.5% Penicillin-Streptomycin, 0.3% BSA, 1% ITS supplement, 100 ng/ml NRG1, 7.5 μM A83-01, 2.5 μM Y27632, and 4% KnockOut Serum Replacement, as described by Okae et al. Cell Stem Cell. 2018 Jan. 4; 22(1):50-63. Matrigel was added to a final concentration of 2%. At day 3, the medium was replaced with the EVT medium without NRG1, and Matrigel was added to a final concentration of 0.5%. At day 6, the cells were either collected for analysis of mRNA expression using qPCR or suspended in the EVT medium without NRG1 and KSR, Matrigel was added to a final concentration of 0.5%, similar to as described in Okae et al. Cell Stem Cell. 2018 Jan. 4; 22(1):50-63, until collection of the cells at day 14. Cells were also seeded on 12-well plates at a density of approximately 10⁵ cells per plate, cultured similarly and fixated in 4% PFA for immunostaining as described above.

Directed differentiation was repeated three times with similar results.

For staining with PI, 1×10⁶ cells from two hbdTSC and two hiTSC lines were seeded on 10 cm plates coated in Matrigel in hTSC medium. Following 2-4 days, medium was switched to BDM. Cells were fixated in ethanol at days 0, 4, 8 for PI staining and stored at −20° C. On staining day, all samples were washed in PBS and resuspended in a staining mixture containing 50 μg/ml RNAse A (Sigma-Aldrich) and 50 μg/ml PI (BD). Following a 30-minutes incubation, cells were analyzed by FACS (Beckman Coulter). Results were analyzed using Kaluza Software.

Formation of trophoblast organoids with bdTSCs and hiTSCs—Similar to as described in Haider et al. Stem Cell Reports. 2018 Aug. 14; 11(2):537-551, bdTSCs and hiTSCs were suspended in trophoblast organoid medium (TOM) composed of DMEM/F12 supplemented with 10 mM HEPES, lx B27, lx N2, mM L-glutamine, 100 ng/mL R-spondin, 1 μM A83-01, 100 ng/mL recombinant human epidermal growth factor (rhEGF), 50 ng/mL recombinant murine hepatocyte growth factor (rmHGF), 2.5 μM prostaglandin E2, 3 μM CHIR99021, and 100 ng/mL Noggin. Growth factor-reduced Matrigel (GFR-M) was added to reach a final concentration of 60%. Solution (40 μL) containing 10⁴-10⁵ bdTSCs/hiTSCs was placed in the center of 24-wells plates. Following 2 minutes at 37° C., the plates were turned upside down to ensure equal spreading of the cells in the solidifying GFR-M-forming domes. Following 15 minutes, the plates were turned again and the domes were carefully overlaid with 500 μL prewarmed TOM. Cells were cultured in 5% oxygen for 10-19 days and then subject to immunostaining.

Immunostaining of bdTSC and hiTSC trophoblast organoids—Organoid-containing Matrigel domes were fixated in 4% PFA overnight. Following, domes were washed twice with PBS for 15 minutes. Domes were submerged in blocking solution containing 3% bovine serum albumin (BSA), 5% fetal bovine serum (FBS), 0.1% Triton X-100 in PBS, at 4° C. overnight. Then, tissues were incubated with primary antibodies including anti-Ki67 (1:200 Abcam, ab15580) and anti-KRT7 (1:200, Abcam, ab215855) diluted in PBS containing 1% BSA and 0.1% Triton X-100, on a rocking plate at 4° C. for two nights. Following, plates were moved to room temperature and continued rocking for at least 2 additional hours prior to washing in PBS containing 0.1% Triton X-100 overnight, with at least 5 changes of buffer. The next day, domes were incubated in secondary antibody solution containing relevant (Alexa) secondary antibody (1:200) diluted in 1% BSA and 0.1% Triton X-100 on a rocking plate at 4° C. overnight. Domes were washed again with PBS containing 0.1% Triton X-100 overnight, with at least 5 changes of buffer. Finally, domes were then incubated with DAPI for 1 hour and stored in PBS in 4° C. until imaging. Imaging was performed using spinning disk confocal microscopy with Nikon Eclipse Ti2 CSU-W1 Yokogawa confocal scanning unit, Andor Zyla sCMOS camera and Nikon Plan Apo VC 20X NA 0.75 lens. Maximal intensity projection images were created using NIS-Elements microscope imaging software.

Engraftment of hiTSCs into NOD-SCID mice and immunohistochemistry (IHC)—For each lesion, approximately 4×10⁶ were trypsinized with TrypLE, washed twice in PBS, resuspended in 150 μl of a 1:2 mixture of Matrigel and PBS and subcutaneously injected into NOD-SCID mice. Lesions were collected nine days after injection, dissected, fixed overnight in 4% paraformaldehyde, embedded in paraffin, sectioned and mounted onto slides. Some slides were stained with H&E, while others were subject to IHC staining. For IHC, slides were deparaffinized in xylene and rehydrated in a decreasing ethanol gradient. Antigen retrieval was performed in a sodium citrate buffer and slides were heated for 3 minutes at 110-120° C. Following a short incubation in 3% hydrogen peroxide, sections were incubated overnight in CAS-block (Invitrogen) with primary antibody anti-KRT7 (1:1000) (Abcam, ab215855). Following, sections were incubated with appropriate HRP-conjugated secondary antibody (Vector Laboratories) for 30 minutes and immunohistochemistry was performed using DAB peroxidase substrate kit (Vector Laboratories). Slides were lightly counterstained with hematoxylin.

TABLE 1
Primers list

| SEQ ID | | | Primer Sequence |
|---|---|---|---|
| NO: | Gene | Application | (5′ -- > 3′) |
| 17 | GAPDH | qPCR analysis of | F: TGGTATCGTGGAAGGACTCA |
| | (intronic) | integration | |
| | | into genomic | |
| | | DNA normalization | |
| 18 | | | R: TTCAGCTCAGGGATGACCTT |
| 19 | GATA3 (F) | qPCR analysis of | AGCCTGTCCTTTGGACCAC |
| | | integration | |
| | | into genomic | |
| | | DNA | |
| 20 | TFAP2C (F) | qPCR analysis of | AACCCTGGAGACCAGAGTCC |
| | | integration | |
| | | into genomic | |
| | | DNA | |
| 21 | ESRRB (F) | qPCR analysis of | GAAAGCATCTCTGGCTCACC |
| | | integration | |
| | | into genomic | |
| | | DNA | |
| 22 | OCT4 (F) | qPCR analysis of | CTGTCTCCGTCACCACTCTG |
| | | integration | |
| | | into genomic | |
| | | DNA | |
| 23 | SOX2 (F) | qPCR analysis of | GCACACTGCCCCTCTCAC |
| | | integration | |
| | | into genomic | |
| | | DNA | |
| 24 | KLF4 (F) | qPCR analysis of | GACCACCTCGCCTTACACAT |
| | | integration | |
| | | into genomic | |
| | | DNA | |
| 25 | MYC (F) | qPCR analysis of | AGCATACATCCTGTCCGTCC |
| | | integration | |
| | | into genomic | |
| | | DNA | |
| 26 | FUW | qPCR analysis of | AGAATACCAGTCAATCTTTCAC |
| | plasmid | integration | |
| | (R) | into genomic | |
| | | DNA | |
| 27 | GAPDH | qPCR analysis of | F: CCTCAACGACCACTTTGTCAAG |
| | | mRNA expression | |
| | | normalization | |
| 28 | | | R: TCTTCCTCTTGTGCTCTTGCTG |
| 29 | GATA3 | qPCR analysis of | F: TCATTAAGCCCAAGCGAAGG |
| | | mRNA expression | |
| 30 | | | R: GTCCCCATTGGCATTCCTC |
| 31 | OCT4 | qPCR analysis of | F: GAGAAGGAGAAGCTGGAGCA |
| | | mRNA expression | |
| 32 | | | R: CTTCTGCTTCAGGAGCTTGG |
| 33 | KLF4 | qPCR analysis of | F: AGGGAGAAGACACTGCGTCA |
| | | mRNA expression | |
| 34 | | | R: AGTCGCTTCATGTGGGAGAG |
| 35 | MYC | qPCR analysis of | F: AGCGACTCTGAGGAGGAACA |
| | | mRNA expression | |
| 36 | | | R: CTCTGACCTTTTGCCAGGAG |
| 37 | GATA3 5 | qPCR analysis of | F: ACGACCCCTCCAAGATAATTTT |
| | UTR | mRNA expression | |
| | (endogenous | | |
| | expression) | | |
| 38 | | | R: GTCGGGGGTCGTTGAATGAT |
| 39 | TFAP2C | qPCR analysis of | F: GGTTGAATCTTCCGGCCG |
| | | mRNA expression | |
| 40 | | | R: TCTGCCACTGGTTTACTAGGA |
| 41 | TFAP2A | qPCR analysis of | F: GGACCACCTGGTATTCTGTATTT |
| | | mRNA expression | |
| 42 | | | R: CTGGGCAACAAAGGACTATGA |
| 43 | GATA2 | qPCR analysis of | F: GAACCGACCACTCATCAAGC |
| | | mRNA expression | |
| 44 | | | R: TTCTTCATGGTCAGTGGCCT |
| 45 | CGB | qPCR analysis of | F: CAGCATCCTATCACCTCCTGGT |
| | | mRNA expression | |
| 46 | | | R: CTGGAACATCTCCATCCTTGGT |
| 47 | KRT7 | qPCR analysis of | F: AAGAACCAGCGTGCCAAGT |
| | | mRNA expression | |
| 48 | | | R: TCCAGCTCCTCCTGCTTG |
| 49 | TP63 | qPCR analysis of | F: AGAAACGAAGATCCCCAGATGA |
| | | mRNA expression | |
| 50 | | | R: CTGTTGCTGTTGCCTGTACGTT |
| 51 | HLA-A | qPCR analysis of | F: GCTCCCACTCCATGAGGTAT |
| | | mRNA expression | |
| 52 | | | R: AGTCTGTGACTGGGCCTTCA |
| 53 | ERVFRD-1 | qPCR analysis of | F: AGCCAACAACATTGACACCA |
| | | mRNA expression | |
| 54 | | | R: TTTGAAGGACTACGGCTGCT |
| 55 | NOTCH1 | qPCR analysis of | F: TTGAATGGTCAATGCGAGTG |
| | | mRNA expression | |
| 56 | | | R: CGCAGAGGGTTGTATTGGTT |
| 57 | CSH1 | qPCR analysis of | F: ACTGGGCAGATCCTCAAGC |
| | | mRNA expression | |
| 58 | | | R: GTCATGGTTGTGCGAGTTTG |
| 58 | PSG1 | qPCR analysis of | F: CTAACCCACCGGCACAGTAT |
| | | mRNA expression | |
| 60 | | | R: TCGACTGTCATGGATTTGGA |
| 61 | HLA-G | qPCR analysis of | F: TTGGGAAGAGGAGACACGGAACAC |
| | | mRNA expression | |
| 62 | | | R: CTCCTTTGTTCAGCCACATTGGCC |
| 63 | MMP2 | qPCR analysis of | F: TGGCACCCATTTACACCTACAC |
| | | mRNA expression | |
| 64 | | | R: ATGTCAGGAGAGGCCCCATAGA |
| 65 | KRT18 | qPCR analysis of | F: CTGCTGCACCTTGAGTCAGA |
| | | mRNA expression | |
| 66 | | | R: ATGTTCAGCAGGGCCTCATA |
| 67 | CDH1 | qPCR analysis of | F: CTCGACACCCGATTCAAAGT |
| | | mRNA expression | |
| 68 | | | R: GGCGTAGACCAAGAAATGGA |
| 69 | OCLN | qPCR analysis of | F: ACAAATGGACCTCTCCTCCA |
| | | mRNA expression | |
| 70 | | | R: ATGGCAATGCACATCACAATA |
| 71 | EPCAM | qPCR analysis of | F: GCAGCTCAGGAAGAATGTGTC |
| | | mRNA expression | |
| 72 | | | R: TGAAGTACACTGGCATTGACG |
| 73 | THY1 | qPCR analysis of | F: CCAGAACGTCACAGTGCTCA |
| | | mRNA expression | |
| 74 | | | R: AGGTGTTCTGAGCCAGCAG |
| 75 | ZEB1 | qPCR analysis of | F: TTTTCCCATTCTGGCTCCTA |
| | | mRNA expression | |
| 76 | | | R: TGGTGATGCTGAAAGAGACG |
| 77 | VIM | qPCR analysis of | F: CCGACACTCCTACAAGATTTAGA |
| | | mRNA expression | |
| 78 | | | R: CAAAGATTTATTGAAGCAGAACC |
| 79 | ACTA2 | qPCR analysis of | F: GTGACGAAGCACAGAGCAAA |
| | | mRNA expression | |
| 80 | | | R: TGGTGATGATGCCATGTTCT |

### Example 1

### Generation of Human Induced Trophoblast Stem Cell-Like Cells (HiTSCs) from Fibroblasts by Ectopic Expression of GATA3, OCT4, KLF4 and c-MYC

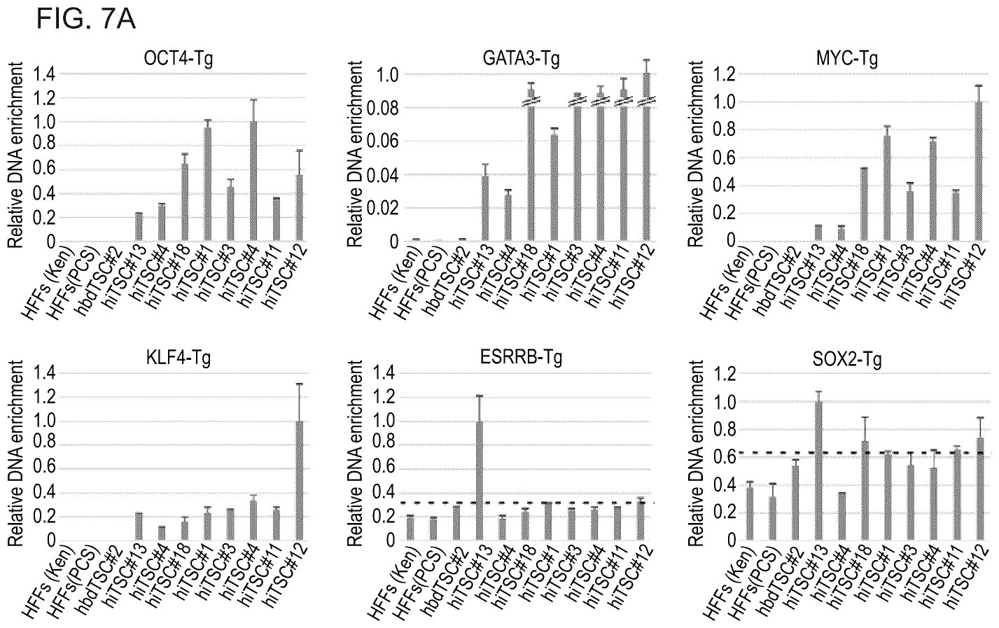

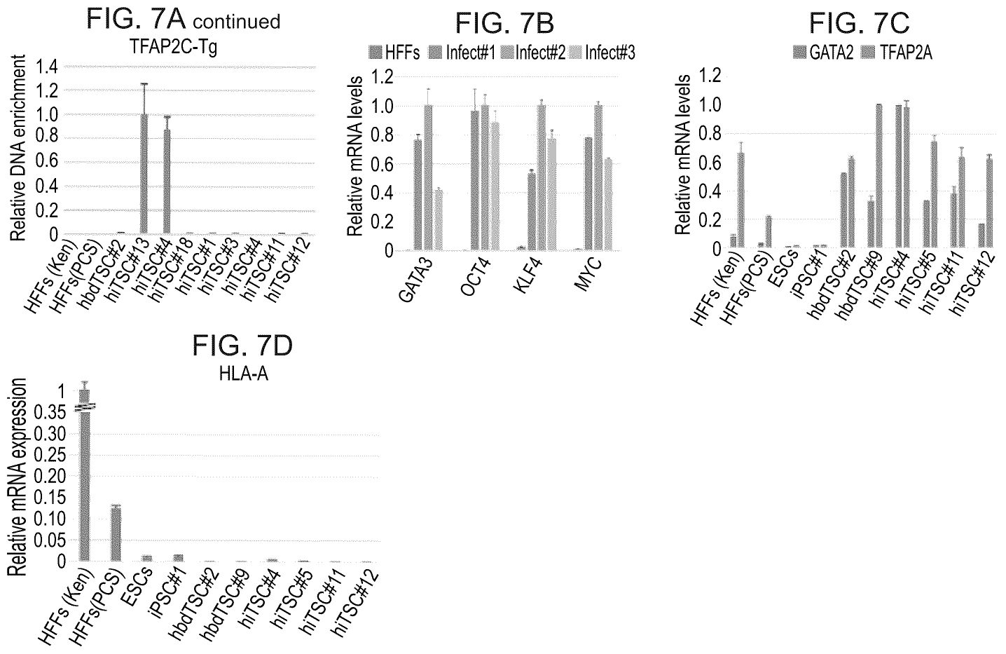

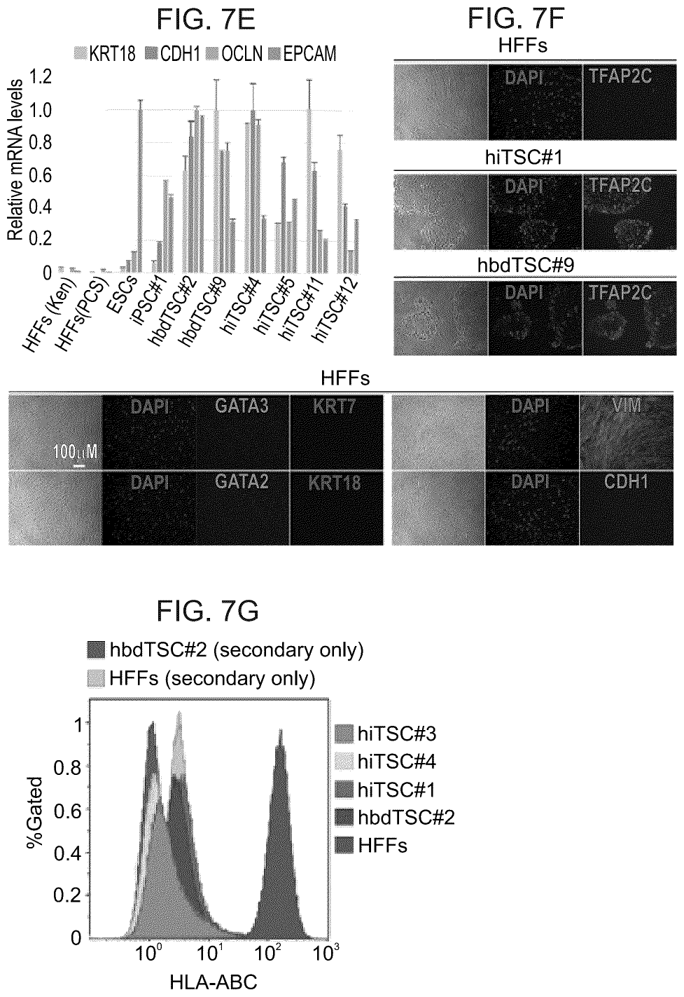

In order to reprogram fibroblasts into human induced trophoblast stem cells (hiTSCs), seven genes, namely GATA3, TFAP2C, ESRRB, OCT4, KLF4, SOX2 and MYC, were cloned into doxycycline (dox)-inducible lentiviral vectors and used to infect human foreskin fibroblasts (HHFs). Cells were kept in low oxygen conditions and treated with dox for two weeks in basic reprogramming medium (DMEM+10% FBS) which was gradually switched to hTSC medium ((Okae et al., 2018), FIG. 1A). Following 4 weeks of reprogramming, the induced cells were weaned off dox and allowed to stabilize for 7-10 days, after which individual epithelial-like colonies were manually transferred into separate plates for propagation and analysis. Transgene integration analysis revealed that GATA3, OCT4, KLF4 and MYC (referred to herein as “GOKM”) were the only transgenes which had been integrated in all examined colonies (FIG. 7A). Indeed, infecting two primary HFF lines, namely KEN and PCS201, with GOKM factors (FIG. 7B) produced stable and dox-independent epithelial-like colonies that exhibited a morphology remarkably similar to that of mTSCs and to human blastocyst-derived TSCs (hbdTSCs) following passaging (FIG. 1B). The reprogramming efficacy ranged between 0.000002-0.00005% depending on infection efficiency, yielding ˜5-100 colonies out of 2×10⁶ seeded HHFs.

In order to evaluate the identity of the resultant colonies, expression of hTSC markers was assessed. Quantitative PCR (qPCR) revealed active transcription of known trophoblast markers such as GATA2, TFAP2A, TFAP2C, KRT7 and TP63, as well as endogenous expression of GATA3, in a manner which is comparable to hbdTSCs (FIGS. 1C and 7C). Furthermore, expression of the HLA class I gene HLA-A was absent from all hiTSC and hbdTSC lines (FIG. 7D). As expected, the resultant hiTSC colonies showed drastic downregulation of mesenchymal markers and upregulation of epithelial markers, indicating successful mesenchymal-to-epithelial transition (MET) (FIGS. 1D and 7E). Of note, the epithelial marker KRT18 discriminated between human epithelial cells from a pluripotency origin (i.e. ESCs and iPSCs) and epithelial cells from a trophectoderm origin (i.e. hbdTSCs and hiTSCs), similarly to mouse (Benchetrit et al., 2015). The expression of hTSC markers, GATA3, GATA2, TFAP2C and KRT7, epithelial markers CDH1 and KRT18, and the absence of the mesenchymal marker VIM, as well as of classical HLA class I proteins (HLA-ABC), was validated at the protein levels as well (FIGS. 1E and 7F-G).

Taken together, these data suggest that transient GOKM expression can force human fibroblasts to become stable and dox-independent epithelial colonies resembling hbdTSCs in their morphology and TSC marker expression.

### Example 2

### Characterization of the Generated hiTSCs

The Transcriptome of hiTSCs is Highly Similar to that of hbdTSCs

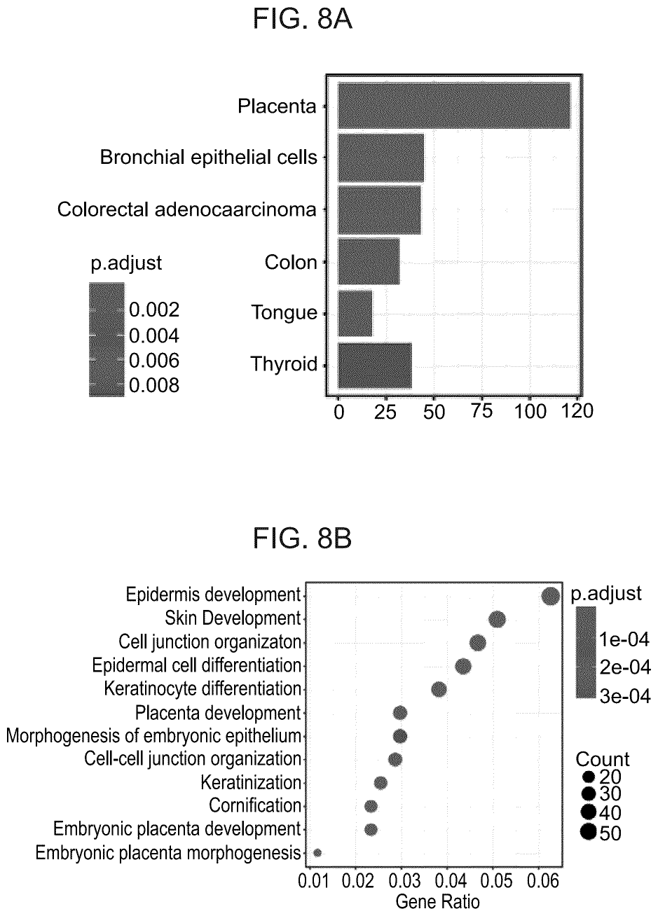

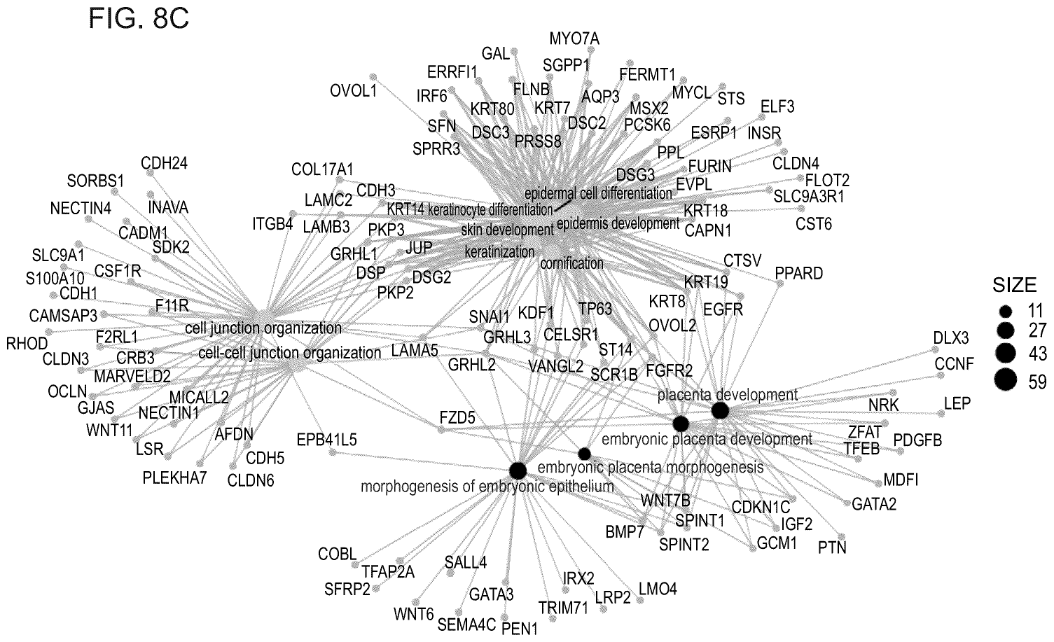

An extensive nuclear reprogramming during somatic cell conversion should result in the activation of the newly established endogenous circuitry of the targeted cells (Buganim et al., 2013; Sebban and Buganim, 2016). Incomplete activation of the endogenous circuitry will lead to a partially similar transcriptome, as can be seen in several direct conversion models (Sebban and Buganim, 2016). To assess whether hiTSCs activated the TSC endogenous circuitry, three hiTSC clones, referred to herein as hiTSC#1, hiTSC#4 and hiTSC#7, were subjected for RNA-sequencing (RNA-seq) analysis. Two hbdTSC lines, referred to herein as hbdTSC#2 and hbdTSC#9, the parental HFFs and hESC/hiPSC lines were used as positive and negative controls, respectively. Notably, the various hiTSC clones clustered together with hbdTSC lines and far away from the HFFs and hESC/hiPSC controls, as indicated by principal component analysis (FIG. 2A) and hierarchical correlation heatmap (FIG. 2B). Of note, the two hbdTSC lines clustered closer to hiTSC clones (i.e. hbdTSC#2 clustered with hiTSC#4 and hbdTSC#9 clustered to hiTSC#7) than to each other (FIG. 2B). Scatter plot analysis indicated highly similar transcriptome between hbdTSCs and hiTSCs with R 2 scored above 0.9 and key hTSC genes such as TP63 and GATA3 were highly expressed in all TSC samples but not in hESC or HFF negative controls (FIG. 2C). Moreover, differentially expressed genes between hTSCs (hbdTSCs or hiTSCs) and hESCs and HFFs revealed significant enrichment for gene ontology terms relevant to placenta and embryonic placenta morphogenesis and development according to the Human Gene Atlas (FIGS. 8A-C).

Taken together, these data suggest that the transcriptome of the induced hTSCs is highly similar to that of the blastocyst-derived hTSCs.

The Methylome and Genomic Integrity of hiTSCs is Comparable to that of hbdTSCs

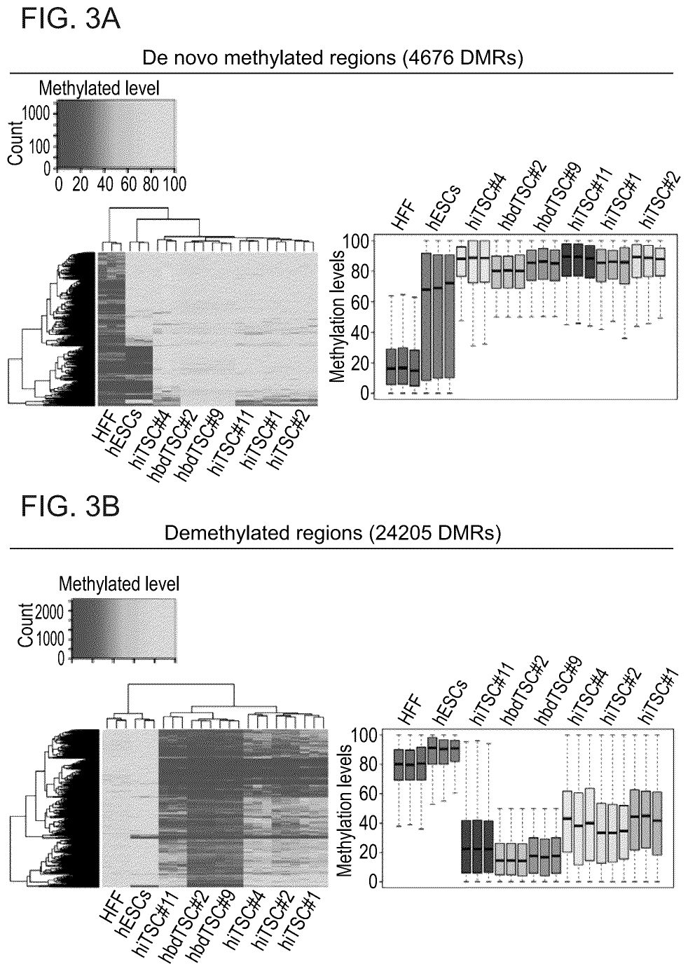

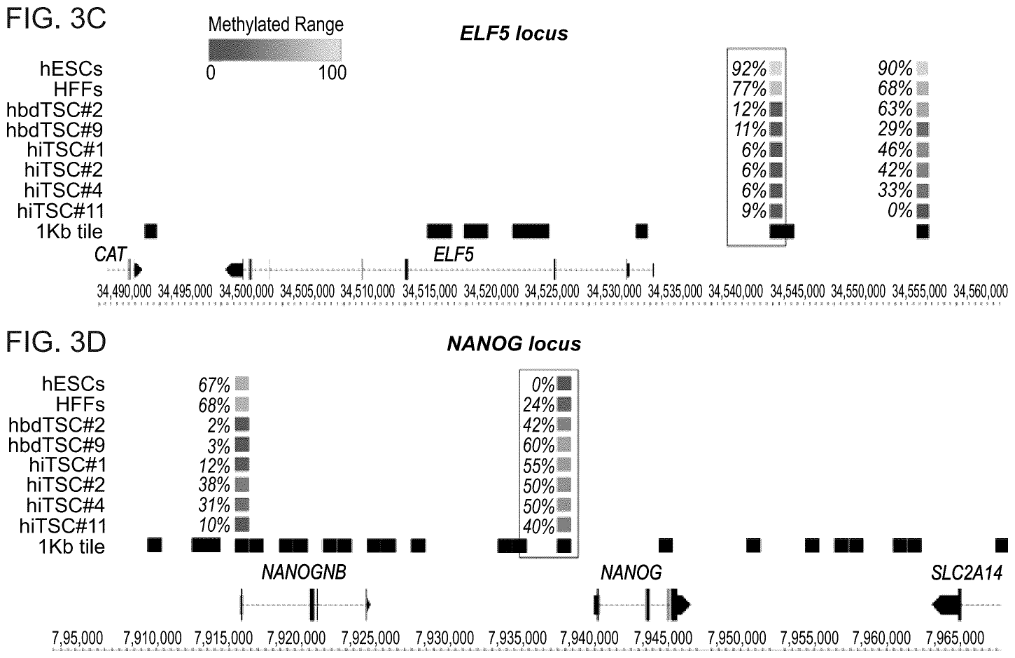

While hiTSC gene expression profile is highly similar to that of hbdTSCs, the present inventors tested whether the epigenetic landscape of hiTSCs is also similar to that of hbdTSCs. One of the epigenetic marks that was shown to be modified during the late stage of OSKM reprogramming to iPSCs is DNA methylation (Apostolou and Hochedlinger, 2013). To test whether the DNA methylation landscape of hiTSCs is equivalent to that of hbdTSCs, four hiTSC clones, referred to herein as hiTSC#1, hiTSC#2, hiTSC#4 and hiTSC#11, were subjected to reduced representation bisulfite sequencing (RRBS) analysis. Two hbdTSC lines, hbdTSC#2 and hbdTSC#9, the parental HFFs and hESC line were used as positive and negative controls, respectively. Methylation analysis revealed 28,881 differentially methylated region (DMRs), while 4676 of them were hypomethylated in HFFs and hypermethylated in the two hbdTSC lines and 24205 DMRs were hypermethylated in HFFs and hypomethylated in the two hbdTSC lines. Notably, analyzing the methylation landscape of the four hiTSC clones revealed that while the 4676 DMRs underwent a robust de novo methylation in all four hiTSC clones (FIG. 3A), the 24205 DMRs showed some variations between the different colonies while some regions remained partially methylated (FIG. 3B). Taken together, these results suggest that demethylation is less rigorous in hiTSC reprogramming Importantly, the overall methylation landscape of hiTSC clones clustered closely to hbdTSCs and far from ESC and HFF controls in both cases of de novo methylated and demethylated DMRs.

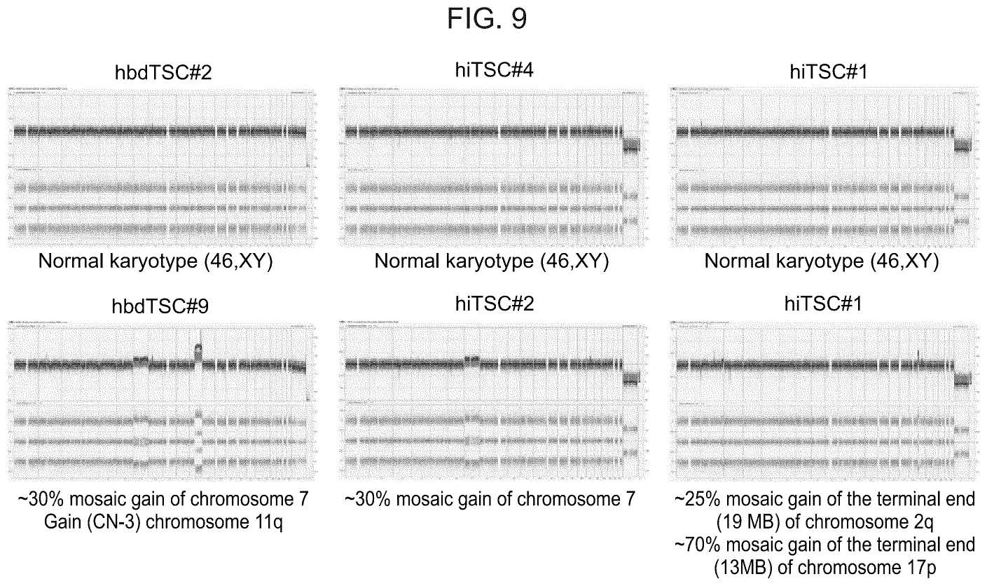

Several gatekeepers were suggested to remain methylated during mouse ESC trans differentiation toward the TSC fate, producing only TS-like cells (Cambuli et al., 2014)). One of these gatekeepers is Elf5. Thus, the present inventors tested whether the ELF5 locus underwent hypomethylation in hiTSCs. Analysis of the RRBS data showed two DMRs in the ELF5 locus while the proximal one (10 kb from the TSS, marked by a square) showed a similar pattern of hypomethylation both in hbdTSCs and in all hiTSCs (FIG. 3C). Similarly, the only DMR that was found in the pluripotency-specific locus NANOG (marked by a square) was completely hypomethylated in ESCs, and to a lesser extent in HFFs, but similarly methylated in both hbdTSCs and hiTSCs (FIG. 3D). Interestingly, another DMR that is located to the adjacent locus, NANOGNB showed a comparable hypomethylation patterns in all hbdTSCs and hiTSCs in contrast to the methylation pattern of HFFs and ESCs (FIG. 3D). Taken together, these data suggest that DNA methylation is largely rewired to the hTSC state in the stable hiTSCs Next, the inventors tested whether the reprogramming process or prolong culture period of hiTSCs is prone to genomic aberrations. To this end, two hbdTSC lines (hbdTSC#2 and hbdTSC#9) and four hiTSC clones were subjected to a sensitive karyotyping measurements using Affymetrix CytoScan 750K array. Thorough analysis revealed that 50% of all colonies from both origin (i.e. hbdTSCs or hiTSCs) harbor an intact karyotype. The other 50% of the colonies exhibited few aberrations in a small fraction of the cells (FIG. 9). These results indicate that hiTSC colonies with intact karyotyping can be isolated and grow in culture and that the reprogramming process does not facilitate genomic instability.

### Example 3

### The Generated hiTSCs Differentiate In-Vitro to all Trophoblast Cell Types

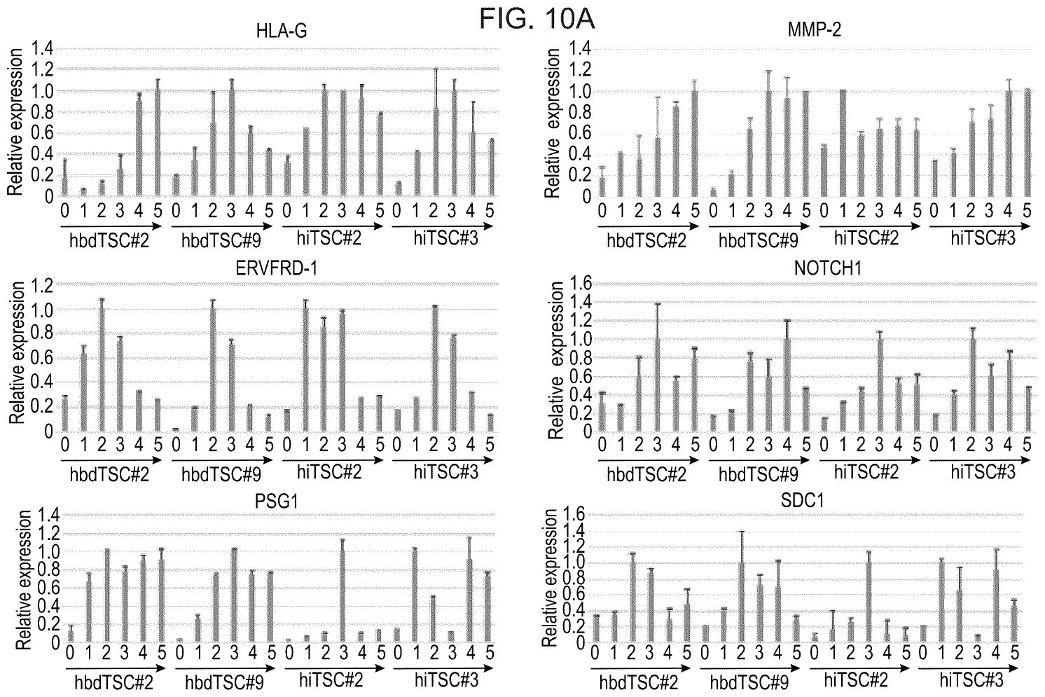

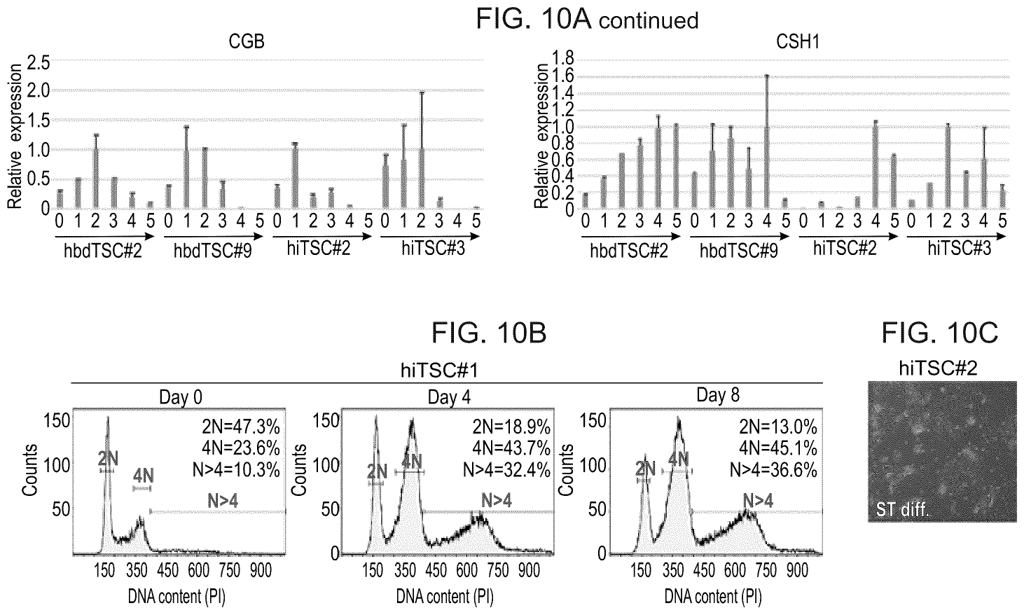

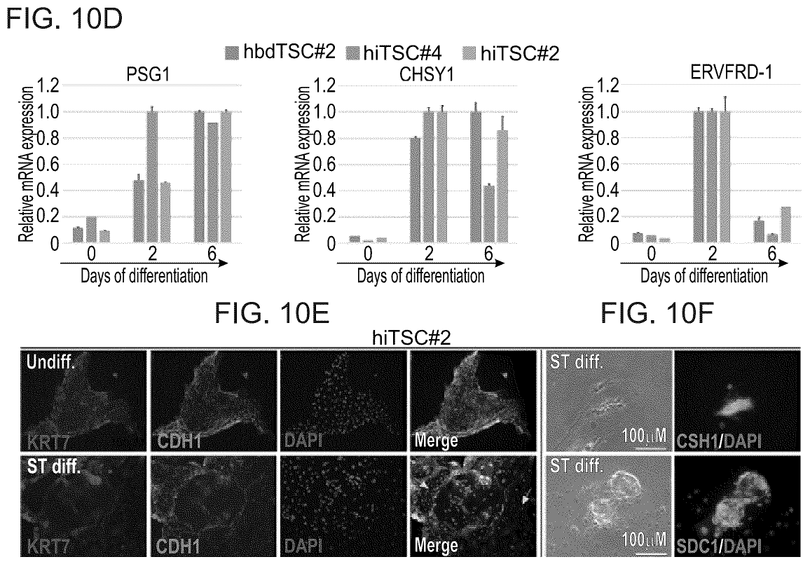

Similarly, to native placental stem cells, hbdTSCs can differentiate into multinucleated syncytium trophoblasts (STs) and extravillous trophoblasts (EVTs) (Okae et al., 2018). Thus, the inventors tested whether hiTSCs hold a similar potential to differentiate into STs and EVTs. Initially, the hTSC culturing medium was replaced into DMEM+10% FBS and the cells were left to differentiate spontaneously. Similar to the mouse, removing the stemness signals from the cells was sufficient to induce spontaneous differentiation into STs and EVTs as assessed by qPCR for EVT (e.g. HLA-G, MMP2 and NOTCH1) and ST-specific markers (e.g. ERVFRD-1, PSG1, SDC1, CGB and CSH1) and PI staining followed by flow cytometry for the present of multinucleated cells (FIGS. 4A and 10A-B).

However, DMEM+10% FBS medium is not optimal for growing trophoblast cells and they tend to die and peel off the plate after several days in DMEM+10% FBS medium. Thus, direct differentiation into STs and EVTs using Okae et al. protocol (Okae et al., 2018) was conducted (FIGS. 4B and 10C). Initially, hbdTSCs and hiTSCs were differentiated into STs, while collecting samples after 2 and 6 days of differentiation. qPCR analysis for ST markers such as ERVFRD-1, CSH1, PSG1 and GCM1 showed a robust induction of ST markers that was equivalent to that of hbdTSCs (FIGS. 4C and 10D). Of note, ERVFRD-1, which orchestrates the fusion event early on in the process, was upregulated after 2 days of differentiation but returned to normal levels once the cells completed the fusion at day 6 Immunostaining for the pan trophoblast, KRT7, the epithelial marker, CDH1 and DAPI showed clear formation of large multinucleated cells after 6 days of differentiation in both hbdTSCs and hiTSCs. As expected, while the undifferentiated hiTSCs stained positive for CDH1, the multinucleated STs were negative to it. SDC1-positive three-dimensional ST structures were observed as well in both cell types (FIGS. 4D-E and 10E-F). These data indicate that hiTSCs hold the capability to differentiate into STs similarly to hbdTSCs.

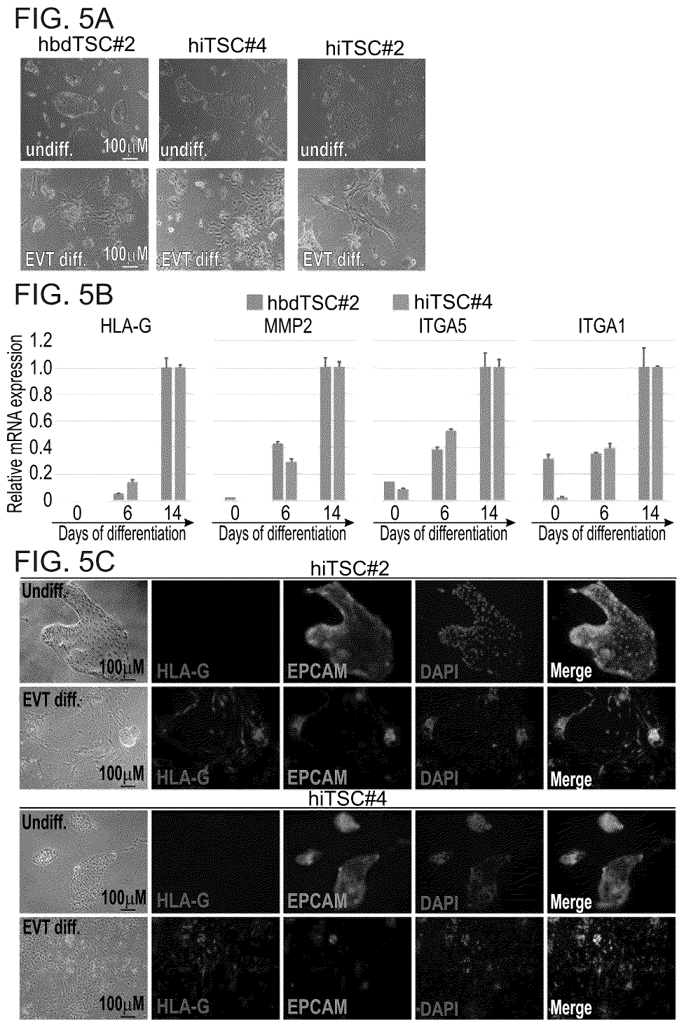

Next, the differentiation of hbdTSCs and hiTSCs into EVTs (Okae et al., 2018) was directed. Following seeding and cell attachment to the plate, cells aggregates were formed in the plates (FIG. 5A). Following six days of differentiation, spindle shape-like cells started to migrate out of the aggregates. qPCR analysis for key EVT genes (e.g. HLA-G and MMP2) (FIG. 5B) and immunostaining (FIG. 5C) validated the identity of the cells as EVTs. Taken together, these results suggest that hiTSCs hold the potential to differentiate into the various cell types of the placenta similarly to hbdTSCs.

### Example 4

### The Generated hiTSCs Proliferate and Differentiate In-Vivo

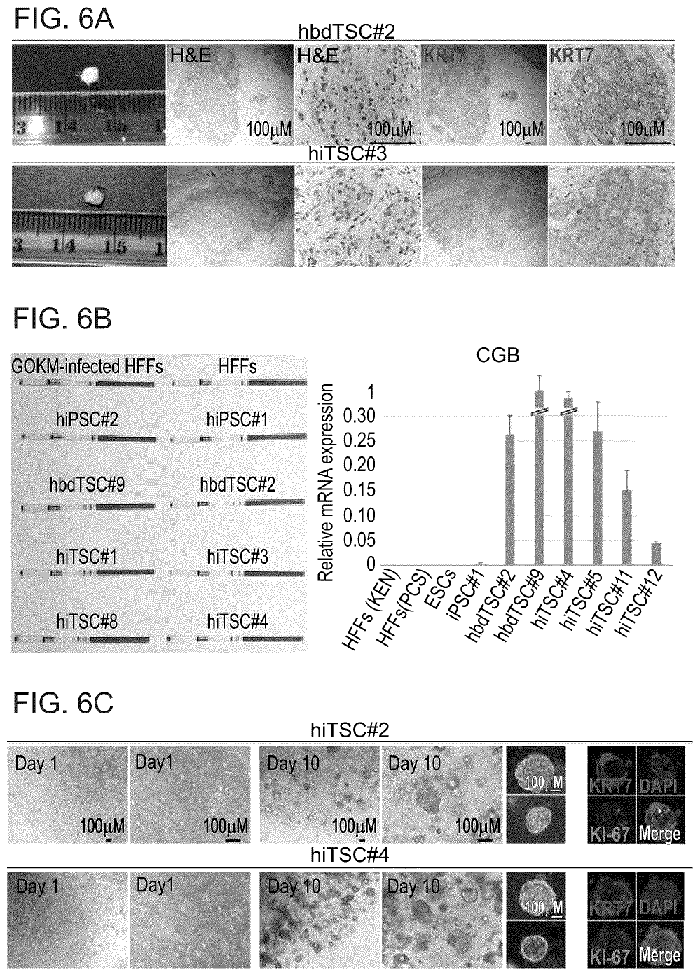

When hbdTSCs are injected subcutaneously into non-obese diabetic (NOD)-severe combined immunodeficiency (SCID) mice the cells form KRT7-positive trophoblastic lesions but with little blood vessel formation (Okae et al., 2018). To test whether hiTSCs are capable of forming KRT7-positive trophoblastic lesions, approximately 4×10⁶ cells from two hiTSC clones (hiTSC#1 and hiTSC#3) and one hbdTSC control line (hbdTSC#2) were subcutaneously injected into NOD/SCID mice. Following 9 days, lesions of approximately 5 mm in size had formed and were extracted (FIG. 6A, FIG. 11) Immunohistochemical staining showed KRT7-positive areas of cells with trophoblast morphology, similarly to previously published findings (Okae et al., 2018). hCG secretion was validated, using an over-the counter pregnancy test, in culture media (FIG. 6B).

### Example 5

### The Generated hiTSCs form Trophoblast Organoids

Recently, two trophoblast organoid systems have been developed and described (Haider et al., 2018; Turco et al., 2018). These studies demonstrated the capability of first trimester villus CTB cells to form 3 dimensional structures that contains both proliferating stem cells and differentiated cells. In depth examination of the two systems revealed that many characteristics of the early developmental program of the human placenta is present also in these organoids. Thus, the present inventors tested whether hiTSCs hold the same potential and from trophoblastic organoids. bdTSC#2 and hiTSC#4 were trypsinized and seeded inside of a drop of matrigel and were left to grow for 10 days. As shown for villus CTBs, both hbdTSCs and hiTSCs were capable of forming 3 dimensional structures within few days of culture Immunostaining for key genes revealed that the organoids consist of both proliferating stem cells and differentiated cells (FIG. 6C). Taken together, these data demonstrate that the hiTSCs can form functional organoids that are similar to their hbdTSC and villus CTBs counterparts.

### Example 6

### Rejuvenating Human Cells by Ectopic Expression of GATA3, OCT4, KLF4/KLF5 and c-MYC

This example provides a non-limiting example of a protocol for rejuvenation. Accordingly, GATA3, OCT4, at least one of KLF4 and KLF5 and optionally c-MYC are introduced as mRNA molecules into CD34+ cells that have been obtained from a human subject, such as a patient suffering from Myelodysplastic syndromes (MDS). Following, the cells are cultured for 1-3 weeks and their epigenetic age and function is examined. Subsequently, the rejuvenated cells are transplanted back to the same patient.

### Example 7

### Generation of hiTSCs from Fibroblasts by Ectopic Expression of GATA3, OCT4, KLF4, KLF5 AND c-MYC

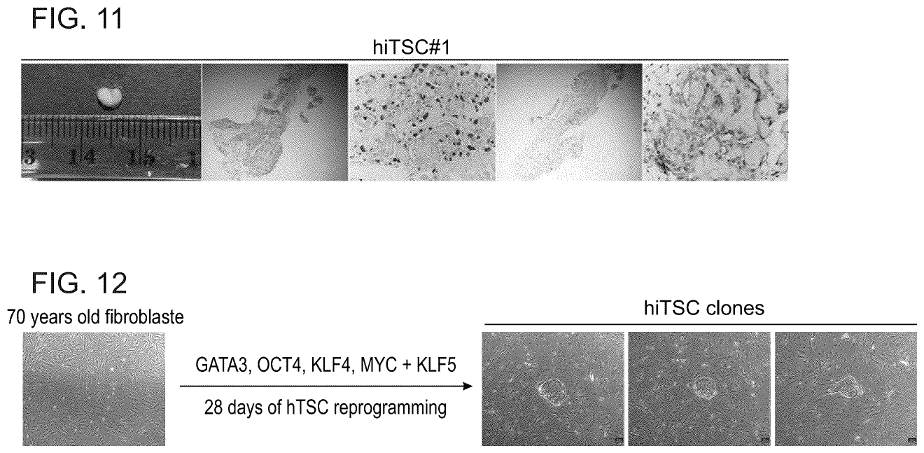

The gene KLF5 was also identified as a strong booster for the reprogramming process to hiTSCs e.g. when combined with GATA3, OCT4, KLF4 and c-MYC. Interestingly, not only that KLF5 facilitated the reprogramming towards hTSCs, it allowed the reprogramming elderly fibroblasts (obtained from PromoCell, Cat no: C-12302) into hiTSCs (FIG. 12).

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

It is the intent of the applicant(s) that all publications, patents and patent applications referred to in this specification are to be incorporated in their entirety by reference into the specification, as if each individual publication, patent or patent application was specifically and individually noted when referenced that it is to be incorporated herein by reference. In addition, citation or identification of any reference in this application shall not be construed as an admission that such reference is available as prior art to the present invention. To the extent that section headings are used, they should not be construed as necessarily limiting. In addition, any priority document(s) of this application is/are hereby incorporated herein by reference in its/their entirety.

### REFERENCES

### Other References are Cited Throughout the Application

Apostolou, E., and Hochedlinger, K. (2013). Chromatin dynamics during cellular reprogramming Nature 502, 462-471.
Benchetrit, H., Herman, S., van Wietmarschen, N., Wu, T., Makedonski, K., Maoz, N., Yom Tov, N., Stave, D., Lasry, R., Zayat, V., et al. (2015). Extensive Nuclear Reprogramming Underlies Lineage Conversion into Functional Trophoblast Stem-like Cells. Cell stem cell 17, 543-556.
Benchetrit, H., Jaber, M., Zayat, V., Sebban, S., Pushett, A., Makedonski, K., Zakheim, Z., Radwan, A., Maoz, N., Lasry, R., et al. (2019). Direct Induction of the Three Pre-implantation Blastocyst Cell Types from Fibroblasts. Cell stem cell 24, 983-994 e987.
Blakeley, P., Fogarty, N. M., Del Valle, I., Wamaitha, S. E., Hu, T. X., Elder, K., Snell, P., Christie, L., Robson, P., and Niakan, K. K. (2015). Defining the three cell lineages of the human blastocyst by single-cell RNA-seq. Development 142, 3613.
Buganim, Y., Faddah, D. A., and Jaenisch, R. (2013). Mechanisms and models of somatic cell reprogramming Nat Rev Genet 14, 427-439.
Cambuli, F., Murray, A., Dean, W., Dudzinska, D., Krueger, F., Andrews, S., Senner, C. E., Cook, S. J., and Hemberger, M. (2014). Epigenetic memory of the first cell fate decision prevents complete ES cell reprogramming into trophoblast. Nature communications 5, 5538.
Deng, Q., Ramskold, D., Reinius, B., and Sandberg, R. (2014). Single-cell RNA-seq reveals dynamic, random monoallelic gene expression in mammalian cells. Science 343, 193-196.
Fogarty, N. M. E., McCarthy, A., Snijders, K. E., Powell, B. E., Kubikova, N., Blakeley, P., Lea, R., Elder, K., Wamaitha, S. E., Kim, D., et al. (2017). Genome editing reveals a role for OCT4 in human embryogenesis. Nature 550, 67-73.
Ghulmiyyah, L., and Sibai, B. (2012). Maternal mortality from preeclampsia/eclampsia. Seminars in perinatology 36, 56-59.
Haider, S., Meinhardt, G., Saleh, L., Kunihs, V., Gamperl, M., Kaindl, U., Ellinger, A., Burkard, T. R., Fiala, C., Pollheimer, J., et al. (2018). Self-Renewing Trophoblast Organoids Recapitulate the Developmental Program of the Early Human Placenta. Stem Cell Reports 11, 537-551.
Harmon, Q. E., Huang, L., Umbach, D. M., Klungsoyr, K., Engel, S. M., Magnus, P., Skjaerven, R., Zhang, J., and Wilcox, A. J. (2015). Risk of fetal death with preeclampsia. Obstetrics and gynecology 125, 628-635.
Krendl, C., Shaposhnikov, D., Rishko, V., Ori, C., Ziegenhain, C., Sass, S., Simon, L., Muller, N. S., Straub, T., Brooks, K. E., et al. (2017). GATA2/3-TFAP2A/C transcription factor network couples human pluripotent stem cell differentiation to trophectoderm with repression of pluripotency. Proceedings of the National Academy of Sciences of the United States of America 114, E9579-E9588.
Latos, P. A., Goncalves, A., Oxley, D., Mohammed, H., Turro, E., and Hemberger, M. (2015). Fgf and Esrrb integrate epigenetic and transcriptional networks that regulate self-renewal of trophoblast stem cells. Nature communications 6, 7776.
Li, Y., Moretto-Zita, M., Soncin, F., Wakeland, A., Wolfe, L., Leon-Garcia, S., Pandian, R., Pizzo, D., Cui, L., Nazor, K., et al. (2013). BMP4-directed trophoblast differentiation of human embryonic stem cells is mediated through a DeltaNp63+cytotrophoblast stem cell state. Development 140, 3965-3976.
Lutomski, J. E., Morrison, J. J., Greene, R. A., and Lydon-Rochelle, M. T. (2011). Maternal morbidity during hospitalization for delivery. Obstetrics and gynecology 117, 596-602.
Okae, H., Toh, H., Sato, T., Hiura, H., Takahashi, S., Shirane, K., Kabayama, Y., Suyama, M., Sasaki, H., and Arima, T. (2018). Derivation of Human Trophoblast Stem Cells. Cell stem cell 22, 50-63 e56.
Petropoulos, S., Edsgard, D., Reinius, B., Deng, Q., Panula, S. P., Codeluppi, S., Reyes, A. P., Linnarsson, S., Sandberg, R., and Lanner, F. (2016). Single-Cell RNA-Seq Reveals Lineage and X Chromosome Dynamics in Human Preimplantation Embryos. Cell 167, 285.
Sebban, S., and Buganim, Y. (2016). Nuclear Reprogramming by Defined Factors: Quantity Versus Quality. Trends Cell Biol 26, 65-75.
Tanaka, S., Kunath, T., Hadjantonakis, A. K., Nagy, A., and Rossant, J. (1998). Promotion of trophoblast stem cell proliferation by FGF4. Science 282, 2072-2075.
Turco, M. Y., Gardner, L., Kay, R. G., Hamilton, R. S., Prater, M., Hollinshead, M. S., McWhinnie, A., Esposito, L., Fernando, R., Skelton, H., et al. (2018). Trophoblast organoids as a model for maternal-fetal interactions during human placentation. Nature 564, 263-267.
van den Berg, D. L., Zhang, W., Yates, A., Engelen, E., Takacs, K., Berstarosti, K., Demmers, J., Chambers, I., and Poot, R. A. (2008). Estrogen-related receptor beta interacts with Oct4 to positively regulate Nanog gene expression. Molecular and cellular biology 28, 5986-5995.
Wallis, A. B., Saftlas, A. F., Hsia, J., and Atrash, H. K. (2008). Secular trends in the rates of preeclampsia, eclampsia, and gestational hypertension, United States, 1987-2004. American journal of hypertension 21, 521-526.
Xu, J., Du, Y., and Deng, H. (2015). Direct lineage reprogramming strategies, mechanisms, and applications. Cell stem cell 16, 119-134.
Zhang, X., Zhang, J., Wang, T., Esteban, M. A., and Pei, D. (2008). Esrrb activates Oct4 transcription and sustains self-renewal and pluripotency in embryonic stem cells. The Journal of biological chemistry 283, 35825-35833.

What is claimed is:

1. A method of generating an induced trophoblast stem cell (iTSC) from a human cell, the method comprising expressing within the cell exogenous GATA3 and OCT4 transcription factors, under conditions which allow generation of an iTSC from said cell, thereby generating the iTSC from the cell.

2. A method of rejuvenating and/or de-differentiating a human cell, the method comprising expressing within the cell exogenous GATA3 and OCT4 transcription factors, under conditions which allow rejuvenation and/or de-differentiation of said cell, thereby generating a rejuvenated cell and/or a de-differentiated cell.

3. The method of claim 1, wherein said expressing comprises transiently expressing.

4. The method of claim 1, further comprising expressing within said cell an exogenous c-MYC transcription factor; and/or an exogenous KLF transcription factor.

5. The method of claim 1, further comprising expressing within said cell an exogenous KLF4 transcription factor.

6. The method of claim 1, wherein said conditions are such that expressing is for at least 14 days following introducing said exogenous transcription factor into said cell; and/or
wherein said conditions are such that expressing is for no more than 30 days following introducing said exogenous transcription factor into said cell.

7. The method of claim 2, wherein said conditions are such that expressing is for at least 1 day following introducing said exogenous transcription factor into said cell; and/or wherein said conditions are such that expressing is for less than 25 days following introducing said exogenous transcription factor into said cell.

8. The method of claim 1, wherein said iTSC does not express said exogenous transcription factor as determined by at least one of PCR, western blot and/or flow cytometry.

9. The method of claim 2, wherein said rejuvenated cell and/or de-differentiated cell does not express said exogenous transcription factor as determined by at least one of PCR, western blot and/or flow cytometry.

10. The method of claim 1, wherein said expressing comprises introducing into said cell a polynucleotide encoding said transcription factor.

11. The method of claim 10, wherein said polynucleotide is a RNA.

12. A nucleic acid construct or system comprising at least one polynucleotide comprising a nucleic acid sequence encoding GATA3 and OCT4 transcription factors.

13. The nucleic acid construct or system of claim 12, wherein said at least one polynucleotide further comprises a nucleic acid sequence encoding a c-MYC transcription factor and/or a KLF transcription factor.

14. The nucleic acid construct or system of claim 12, wherein said at least one polynucleotide further comprises a nucleic acid sequence encoding a KLF4 transcription factor.

15. The nucleic acid construct or system of claim 12, wherein said at least one polynucleotide is a RNA.

16. A protein preparation comprising GATA3 and OCT4 transcription factors polypeptides to a level of purity of at least 20%.

17. The protein preparation of claim 16, further comprising a c-MYC transcription factor polypeptide; and/or a KLF transcription factor polypeptide.

18. The protein preparation of claim 16, further comprising KLF4 transcription factor polypeptide.

19. An isolated human cell expressing exogenous GATA3 and OCT4 transcription factors.

20. The isolated cell of claim 19, further expressing an exogenous c-MYC transcription factor; and/or exogenous KLF transcription factor.

21. The isolated cell of claim 19, further expressing an exogenous KLF4 transcription factor.

22. An isolated induced trophoblast stem cell (iTSC) obtainable according to the method of claim 1.

23. An isolated rejuvenated and/or de-differentiated cell obtainable according to the method of claim 1.

24. An isolated population of cells, wherein at least 80% of said cells are the iTSCs of claim 22.

25. An isolated population of cells, wherein at least 80% of said cells are the rejuvenated and/or de-differentiated cells of claim 23.

26. A method of augmenting a placenta, a developing embryo or a synthetic embryo comprising introducing into a placenta, a developing embryo or a synthetic embryo the iTSC of claim 22.

27. A method of generating an aggregate or organoid comprising trophoblasts, the method comprising introducing into a scaffold or a matrix the iTSC of claim 22.

28. A method of treating and/or preventing a disorder associated with development and/or activity of trophoblasts in a subject in need thereof, the method comprising administering to the subject a therapeutically effective amount of the iTSC of claim 22, thereby treating and/or preventing the disorder associated with development and/or activity of trophoblasts in the subject.

29. A method of treating and/or preventing a disease associated with aging in a subject in need thereof, the method comprising administering to the subject a therapeutically effective amount of the cell of claim 23, thereby treating and/or preventing the disease in the subject.

30. A method of performing a cosmetic care in a subject in need thereof, the method comprising applying to the skin of the subject a therapeutically effective amount of the protein preparation of claim 16, thereby performing the cosmetic care.


SEQUENCE LISTING (93 sequences)

SEQ ID NO: 1 - protein, 444 aa, Homo sapiens
MEVTADQPRWVSHHHPAVLNGQHPDTHHPGLSHSYMDAAQYPLPEEVDVLFNIDGQGNHVPPYYGNSVRATVQRYPPTHHGSQVCRPPLLHGSLPWLDGGKALGSHHTASPWNLSPFSKTSIHHGSPGPLSVYPPASSSSLSGGHASPHLFTFPPTPPKDVSPDPSLSTPGSAGSARQDEKECLKYQVPLPDSMKLESSHSRGSMTALGGASSSTHHPITTYPPYVPEYSSGLFPPSSLLGGSPTGFGCKSRPKARSSTEGRECVNCGATSTPLWRRDGTGHYLCNACGLYHKMNGQNRPLIKPKRRLSAARRAGTSCANCQTTTTTLWRRNANGDPVCNACGLYYKLHNINRPLTMKKEGIQTRNRKMSSKSKKCKKVHDSLEDFPKNSSFNPAALSRHMSSLSHISPFSHSSHMLTTPTPMHPPSSLSFGPHHPSSMVTAMG

SEQ ID NO: 2 - DNA, 3083 bp, Homo sapiens
  source 1..3083: mol_type = genomic DNA
gaacactgagctgcctggcgccgtcttgatactttcagaaagaatgcattccctgtaaaaaaaaaaaaaaaatactgagagagggagagagagagagaagaagagagagagacggagggagagcgagacagagcgagcaacgcaatctgaccgagcaggtcgtacgccgccgcctcctcctcctctctgctcttcgctacccaggtgacccgaggagggactccgcctccgagcggctgaggaccccggtgcagaggagcctggctcgcagaattgcagagtcgtcgcccctttttacaacctggtcccgttttattctgccgtacccagtttttggatttttgtcttccccttcttctctttgctaaacgacccctccaagataatttttaaaaaaccttctcctttgctcacctttgcttcccagccttcccatccccccaccgaaagcaaatcattcaacgacccccgaccctccgacggcaggagccccccgacctcccaggcggaccgccctccctccccgcgcgcgggttccgggcccggcgagagggcgcgagcacagccgaggccatggaggtgacggcggaccagccgcgctgggtgagccaccaccaccccgccgtgctcaacgggcagcacccggacacgcaccacccgggcctcagccactcctacatggacgcggcgcagtacccgctgccggaggaggtggatgtgctttttaacatcgacggtcaaggcaaccacgtcccgccctactacggaaactcggtcagggccacggtgcagaggtaccctccgacccaccacgggagccaggtgtgccgcccgcctctgcttcatggatccctaccctggctggacggcggcaaagccctgggcagccaccacaccgcctccccctggaatctcagccccttctccaagacgtccatccaccacggctccccggggcccctctccgtctaccccccggcctcgtcctcctccttgtcggggggccacgccagcccgcacctcttcaccttcccgcccaccccgccgaaggacgtctccccggacccatcgctgtccaccccaggctcggccggctcggcccggcaggacgagaaagagtgcctcaagtaccaggtgcccctgcccgacagcatgaagctggagtcgtcccactcccgtggcagcatgaccgccctgggtggagcctcctcgtcgacccaccaccccatcaccacctacccgccctacgtgcccgagtacagctccggactcttcccccccagcagcctgctgggcggctcccccaccggcttcggatgcaagtccaggcccaaggcccggtccagcacagaaggcagggagtgtgtgaactgtggggcaacctcgaccccactgtggcggcgagatggcacgggacactacctgtgcaacgcctgcgggctctatcacaaaatgaacggacagaaccggcccctcattaagcccaagcgaaggctgtctgcagccaggagagcagggacgtcctgtgcgaactgtcagaccaccacaaccacactctggaggaggaatgccaatggggaccctgtctgcaatgcctgtgggctctactacaagcttcacaatattaacagacccctgactatgaagaaggaaggcatccagaccagaaaccgaaaaatgtctagcaaatccaaaaagtgcaaaaaagtgcatgactcactggaggacttccccaagaacagctcgtttaacccggccgccctctccagacacatgtcctccctgagccacatctcgcccttcagccactccagccacatgctgaccacgcccacgccgatgcacccgccatccagcctgtcctttggaccacaccacccctccagcatggtcaccgccatgggttagagccctgctcgatgctcacagggcccccagcgagagtccctgcagtccctttcgacttgcatttttgcaggagcagtatcatgaagcctaaacgcgatggatatatgtttttgaaggcagaaagcaaaattatgtttgccactttgcaaaggagctcactgtggtgtctgtgttccaaccactgaatctggaccccatctgtgaataagccattctgactcatatcccctatttaacagggtctctagtgctgtgaaaaaaaaaatgctgaacattgcatataacttatattgtaagaaatactgtacaatgactttattgcatctgggtagctgtaaggcatgaaggatgccaagaagtttaaggaatatgggagaaatagtgtggaaattaagaagaaactaggtctgatattcaaatggacaaactgccagttttgtttcctttcactggccacagttgtttgatgcattaaaagaaaataaaaaaaagaaaaaagagaaaagaaaaaaaaagaaaaaagttgtaggcgaatcatttgttcaaagctgttggcctctgcaaaggaaataccagttctgggcaatcagtgttaccgttcaccagttgccgttgagggtttcagagagcctttttctaggcctacatgctttgtgaacaagtccctgtaattgttgtttgtatgtataattcaaagcaccaaaataagaaaagatgtagatttatttcatcatattatacagaccgaactgttgtataaatttatttactgctagtcttaagaactgctttctttcgtttgtttgtttcaatattttccttctctctcaatttttggttgaataaactagattacattcagttggcctaaggtggttgtgctcggagggtttcttgtttcttttccattttgtttttggatgatatttattaaatagcttctaagagtccggcggcatctgtcttgtccctattcctgcagcctgtgctgagggtagcagtgtatgagctaccagcgtgcatgtcagcgaccctggcccgacaggccacgtcctgcaatcggcccggctgcctcttcgccctgtcgtgttctgtgttagtgatcactgcctttaatacagtctgttggaataatattataagcataataataaagtgaaaatattttaaaacta

SEQ ID NO: 3 - protein, 190 aa, Homo sapiens
MGVLFGKVFSQTTICRFEALQLSFKNMCKLRPLLQKWVEEADNNENLQEICKAETLVQARKRKRTSIENRVRGNLENLFLQCPKPTLQQISHIAQQLGLEKDVVRVWFCNRRQKGKRSSSDYAQREDFEAAGSPFSGGPVSFPLAPGPHFGTPGYGSPHFTALYSSVPFPEGEAFPPVSVTTLGSPMHSN

SEQ ID NO: 4 - protein, 164 aa, Homo sapiens
MCKLRPLLQKWVEEADNNENLQEICKAETLVQARKRKRTSIENRVRGNLENLFLQCPKPTLQQISHIAQQLGLEKDVVRVWFCNRRQKGKRSSSDYAQREDFEAAGSPFSGGPVSFPLAPGPHFGTPGYGSPHFTALYSSVPFPEGEAFPPVSVTTLGSPMHSN

SEQ ID NO: 5 - protein, 265 aa, Homo sapiens
MHFYRLFLGATRRFLNPEWKGEIDNWCVYVLTSLLPFKIQSQDIKALQKELEQFAKLLKQKRITLGYTQADVGLTLGVLFGKVFSQTTICRFEALQLSFKNMCKLRPLLQKWVEEADNNENLQEICKAETLVQARKRKRTSIENRVRGNLENLFLQCPKPTLQQISHIAQQLGLEKDVVRVWFCNRRQKGKRSSSDYAQREDFEAAGSPFSGGPVSFPLAPGPHFGTPGYGSPHFTALYSSVPFPEGEAFPPVSVTTLGSPMHSN

SEQ ID NO: 6 - protein, 360 aa, Homo sapiens
MAGHLASDFAFSPPPGGGGDGPGGPEPGWVDPRTWLSFQGPPGGPGIGPGVGPGSEVWGIPPCPPPYEFCGGMAYCGPQVGVGLVPQGGLETSQPEGEAGVGVESNSDGASPEPCTVTPGAVKLEKEKLEQNPEESQDIKALQKELEQFAKLLKQKRITLGYTQADVGLTLGVLFGKVFSQTTICRFEALQLSFKNMCKLRPLLQKWVEEADNNENLQEICKAETLVQARKRKRTSIENRVRGNLENLFLQCPKPTLQQISHIAQQLGLEKDVVRVWFCNRRQKGKRSSSDYAQREDFEAAGSPFSGGPVSFPLAPGPHFGTPGYGSPHFTALYSSVPFPEGEAFPPVSVTTLGSPMHSN

SEQ ID NO: 7 - protein, 190 aa, Homo sapiens; identical to SEQ ID NO: 3

SEQ ID NO: 8 - DNA, 2075 bp, Homo sapiens
  source 1..2075: mol_type = genomic DNA
ggaaaaaaggaaagtgcacttggaagagatccaagtgggcaacttgaagaacaagtgccaaatagcacttctgtcatgctggatgtcagggctctttgtccactttgtatagccgctggcttatagaaggtgctcgataaatctcttgaatttaaaaatcaattaggatgcctctatagtgaaaaagatacagtaaagatgagggataatcaatttaaaaaatgagtaagtacacacaaagcactttatccattcttatgacacctgttacttttttgctgtgtttgtgtgtatgcatgccatgttatagtttgtgggaccctcaaagcaagctggggagagtatatactgaatttagcttctgagacatgatgctcttcctttttaattaacccagaacttagcagcttatctatttctctaatctcaaaacatccttaaactgggggtgatacttgagtgagagaattttgcaggtattaaatgaactatcttcttttttttttttctttgagacagagtcttgctctgtcacccaggctggagtgcagtggcgtgatctcagctcactgcaacctccgcctcccgggttcaagtgattctcctgcctcagcctcctgagtagctgggattacaggtgcgtgccaccgtgcccagctaatttttgtgtttttagtagagacggggtttcaccatgttggccatgctggtcttgaactcctgacctcgtgatctgcccacctcggcctcccaaagtgctggaattataggcgtgagccaccgcgcccagcaaagaacttctaaccttcataacctgacaggtgttctcgaggccagggtctctctttctgtcctttcacgatgctctgcatcccttggatgtgccagtttctgggggaagagtagtcctttgttacatgcatgagtcagtgaacagggaatgggtgaatgacatttgtgggtaggttatttctagaagttaggtgggcagcttggaaggcagaggcacttctacagactattccttggggccacacgtaggttcttgaatcccgaatggaaaggggagattgataactggtgtgtttatgttcttacaagtcttctgccttttaaaatccagtcccaggacatcaaagctctgcagaaagaactcgagcaatttgccaagctcctgaagcagaagaggatcaccctgggatatacacaggccgatgtggggctcaccctgggggttctatttgggaaggtattcagccaaacgaccatctgccgctttgaggctctgcagcttagcttcaagaacatgtgtaagctgcggcccttgctgcagaagtgggtggaggaagctgacaacaatgaaaatcttcaggagatatgcaaagcagaaaccctcgtgcaggcccgaaagagaaagcgaaccagtatcgagaaccgagtgagaggcaacctggagaatttgttcctgcagtgcccgaaacccacactgcagcagatcagccacatcgcccagcagcttgggctcgagaaggatgtggtccgagtgtggttctgtaaccggcgccagaagggcaagcgatcaagcagcgactatgcacaacgagaggattttgaggctgctgggtctcctttctcagggggaccagtgtcctttcctctggccccagggccccattttggtaccccaggctatgggagccctcacttcactgcactgtactcctcggtccctttccctgagggggaagcctttccccctgtctccgtcaccactctgggctctcccatgcattcaaactgaggtgcctgcccttctaggaatgggggacagggggaggggaggagctagggaaagaaaacctggagtttgtgccagggtttttgggattaagttcttcattcactaaggaaggaattgggaacacaaagggtgggggcaggggagtttggggcaactggttggagggaaggtgaagttcaatgatgctcttgattttaatcccacatcatgtatcacttttttcttaaataaagaagcctgggacacagtagatagacacacttaaaaaaaaaaa

SEQ ID NO: 9 - DNA, 1589 bp, Homo sapiens
  source 1..1589: mol_type = genomic DNA
ggaaaaaaggaaagtgcacttggaagagatccaagtgggcaacttgaagaacaagtgccaaatagcacttctgtcatgctggatgtcagggctctttgtccactttgtatagccgctggcttatagaaggtgctcgataaatctcttgaatttaaaaatcaattaggatgcctctatagtgaaaaagatacagtaaagatgagggataatcaatttaaaaaatgagtaagtacacacaaagcactttatccattcttatgacacctgttacttttttgctgtgtttgtgtgtatgcatgccatgttatagtttgtgggaccctcaaagcaagctggggagagtatatactgaatttagcttctgagacatgatgctcttcctttttaattaacccagaacttagcagcttatctatttctctaatctcaaaacatccttaaactgggggtgatacttgagtgagagaattttgcaggtattaaatgaactatcttcttttttttttttctttgagacagagtcttgctctgtcacccaggctggagtgcagtggcgtgatctcagctcactgcaacctccgcctcccgggttcaagtgattctcctgcctcagcctcctgagtagctgggattacagtcccaggacatcaaagctctgcagaaagaactcgagcaatttgccaagctcctgaagcagaagaggatcaccctgggatatacacaggccgatgtggggctcaccctgggggttctatttgggaaggtattcagccaaacgaccatctgccgctttgaggctctgcagcttagcttcaagaacatgtgtaagctgcggcccttgctgcagaagtgggtggaggaagctgacaacaatgaaaatcttcaggagatatgcaaagcagaaaccctcgtgcaggcccgaaagagaaagcgaaccagtatcgagaaccgagtgagaggcaacctggagaatttgttcctgcagtgcccgaaacccacactgcagcagatcagccacatcgcccagcagcttgggctcgagaaggatgtggtccgagtgtggttctgtaaccggcgccagaagggcaagcgatcaagcagcgactatgcacaacgagaggattttgaggctgctgggtctcctttctcagggggaccagtgtcctttcctctggccccagggccccattttggtaccccaggctatgggagccctcacttcactgcactgtactcctcggtccctttccctgagggggaagcctttccccctgtctccgtcaccactctgggctctcccatgcattcaaactgaggtgcctgcccttctaggaatgggggacagggggaggggaggagctagggaaagaaaacctggagtttgtgccagggtttttgggattaagttcttcattcactaaggaaggaattgggaacacaaagggtgggggcaggggagtttggggcaactggttggagggaaggtgaagttcaatgatgctcttgattttaatcccacatcatgtatcacttttttcttaaataaagaagcctgggacacagtagatagacacacttaaaaaaaaaaa

SEQ ID NO: 10 - DNA, 2300 bp, Homo sapiens
  source 1..2300: mol_type = genomic DNA
ggaaaaaaggaaagtgcacttggaagagatccaagtgggcaacttgaagaacaagtgccaaatagcacttctgtcatgctggatgtcagggctctttgtccactttgtatagccgctggcttatagaaggtgctcgataaatctcttgaatttaaaaatcaattaggatgcctctatagtgaaaaagatacagtaaagatgagggataatcaatttaaaaaatgagtaagtacacacaaagcactttatccattcttatgacacctgttacttttttgctgtgtttgtgtgtatgcatgccatgttatagtttgtgggaccctcaaagcaagctggggagagtatatactgaatttagcttctgagacatgatgctcttcctttttaattaacccagaacttagcagcttatctatttctctaatctcaaaacatccttaaactgggggtgatacttgagtgagagaattttgcaggtattaaatgaactatcttcttttttttttttctttgagacagagtcttgctctgtcacccaggctggagtgcagtggcgtgatctcagctcactgcaacctccgcctcccgggttcaagtgattctcctgcctcagcctcctgagtagctgggattacaggtgcgtgccaccgtgcccagctaatttttgtgtttttagtagagacggggtttcaccatgttggccatgctggtcttgaactcctgacctcgtgatctgcccacctcggcctcccaaagtgctggaattataggcgtgagccaccgcgcccagcaaagaacttctaaccttcataacctgacaggtgttctcgaggccagggtctctctttctgtcctttcacgatgctctgcatcccttggatgtgccagtttctgggggaagagtagtcctttgttacatgcatgagtcagtgaacagggaatgggtgaatgacatttgtgggtaggttatttctagaagttaggtgggcagcttggaaggcagaggcacttctacagactattccttggggccacacgtaggttcttgaatcccgaatggaaaggggagattgataactggtgtgtttatgttcttacaagtcttctgccttttaaaatccagtcccaggacatcaaagctctgcagaaagaactcgagcaatttgccaagctcctgaagcagaagaggatcaccctgggatatacacaggccgatgtggggctcaccctgggggttctatttggtgggttcccctctgcagattctgaccgcatctcccctctaaggagtatccctgaacctagtggggaggggcaggggcagactaccctcacccatgaagaggagtagggagagggagaagatgctttgagctccctctgggaagaggtggtaagcttggatctcagggtcacaagggccctgcgtgctccctcactttgcttctcttttgactggcctcccccagggaaggtattcagccaaacgaccatctgccgctttgaggctctgcagcttagcttcaagaacatgtgtaagctgcggcccttgctgcagaagtgggtggaggaagctgacaacaatgaaaatcttcaggagatatgcaaagcagaaaccctcgtgcaggcccgaaagagaaagcgaaccagtatcgagaaccgagtgagaggcaacctggagaatttgttcctgcagtgcccgaaacccacactgcagcagatcagccacatcgcccagcagcttgggctcgagaaggatgtggtccgagtgtggttctgtaaccggcgccagaagggcaagcgatcaagcagcgactatgcacaacgagaggattttgaggctgctgggtctcctttctcagggggaccagtgtcctttcctctggccccagggccccattttggtaccccaggctatgggagccctcacttcactgcactgtactcctcggtccctttccctgagggggaagcctttccccctgtctccgtcaccactctgggctctcccatgcattcaaactgaggtgcctgcccttctaggaatgggggacagggggaggggaggagctagggaaagaaaacctggagtttgtgccagggtttttgggattaagttcttcattcactaaggaaggaattgggaacacaaagggtgggggcaggggagtttggggcaactggttggagggaaggtgaagttcaatgatgctcttgattttaatcccacatcatgtatcacttttttcttaaataaagaagcctgggacacagtagatagacacacttaaaaaaaaaaa

SEQ ID NO: 11 - DNA, 2075 bp, Homo sapiens
  source 1..2075: mol_type = genomic DNA
ggaaaaaaggaaagtgcacttggaagagatccaagtgggcaacttgaagaacaagtgccaaatagcacttctgtcatgctggatgtcagggctctttgtccactttgtatagccgctggcttatagaaggtgctcgataaatctcttgaatttaaaaatcaattaggatgcctctatagtgaaaaagatacagtaaagatgagggataatcaatttaaaaaatgagtaagtacacacaaagcactttatccattcttatgacacctgttacttttttgctgtgtttgtgtgtatgcatgccatgttatagtttgtgggaccctcaaagcaagctggggagagtatatactgaatttagcttctgagacatgatgctcttcctttttaattaacccagaacttagcagcttatctatttctctaatctcaaaacatccttaaactgggggtgatacttgagtgagagaattttgcaggtattaaatgaactatcttcttttttttttttctttgagacagagtcttgctctgtcacccaggctggagtgcagtggcgtgatctcagctcactgcaacctccgcctcccgggttcaagtgattctcctgcctcagcctcctgagtagctgggattacaggtgcgtgccaccgtgcccagctaatttttgtgtttttagtagagacggggtttcaccatgttggccatgctggtcttgaactcctgacctcgtgatctgcccacctcggcctcccaaagtgctggaattataggcgtgagccaccgcgcccagcaaagaacttctaaccttcataacctgacaggtgttctcgaggccagggtctctctttctgtcctttcacgatgctctgcatcccttggatgtgccagtttctgggggaagagtagtcctttgttacatgcatgagtcagtgaacagggaatgggtgaatgacatttgtgggtaggttatttctagaagttaggtgggcagcttggaaggcagatgcacttctacagactattccttggggccacacgtaggttcttgaatcccgaatggaaaggggagattgataactggtgtgtttatgttcttacaagtcttctgccttttaaaatccagtcccaggacatcaaagctctgcagaaagaactcgagcaatttgccaagctcctgaagcagaagaggatcaccctgggatatacacaggccgatgtggggctcaccctgggggttctatttgggaaggtattcagccaaacgaccatctgccgctttgaggctctgcagcttagcttcaagaacatgtgtaagctgcggcccttgctgcagaagtgggtggaggaagctgacaacaatgaaaatcttcaggagatatgcaaagcagaaaccctcgtgcaggcccgaaagagaaagcgaaccagtatcgagaaccgagtgagaggcaacctggagaatttgttcctgcagtgcccgaaacccacactgcagcagatcagccacatcgcccagcagcttgggctcgagaaggatgtggtccgagtgtggttctgtaaccggcgccagaagggcaagcgatcaagcagcgactatgcacaacgagaggattttgaggctgctgggtctcctttctcagggggaccagtgtcctttcctctggccccagggccccattttggtaccccaggctatgggagccctcacttcactgcactgtactcctcggtccctttccctgagggggaagcctttccccctgtctccgtcaccactctgggctctcccatgcattcaaactgaggtgcctgcccttctaggaatgggggacagggggaggggaggagctagggaaagaaaacctggagtttgtgccagggtttttgggattaagttcttcattcactaaggaaggaattgggaacacaaagggtgggggcaggggagtttggggcaactggttggagggaaggtgaagttcaatgatgctcttgattttaatcccacatcatgtatcacttttttcttaaataaagaagcctgggacacagtagatagacacacttaaaaaaaaaaa

SEQ ID NO: 12 - DNA, 1409 bp, Homo sapiens
  source 1..1409: mol_type = genomic DNA
gagtagtcccttcgcaagccctcatttcaccaggcccccggcttggggcgccttccttccccatggcgggacacctggcttcggatttcgccttctcgccccctccaggtggtggaggtgatgggccaggggggccggagccgggctgggttgatcctcggacctggctaagcttccaaggccctcctggagggccaggaatcgggccgggggttgggccaggctctgaggtgtgggggattcccccatgccccccgccgtatgagttctgtggggggatggcgtactgtgggccccaggttggagtggggctagtgccccaaggcggcttggagacctctcagcctgagggcgaagcaggagtcggggtggagagcaactccgatggggcctccccggagccctgcaccgtcacccctggtgccgtgaagctggagaaggagaagctggagcaaaacccggaggagtcccaggacatcaaagctctgcagaaagaactcgagcaatttgccaagctcctgaagcagaagaggatcaccctgggatatacacaggccgatgtggggctcaccctgggggttctatttgggaaggtattcagccaaacgaccatctgccgctttgaggctctgcagcttagcttcaagaacatgtgtaagctgcggcccttgctgcagaagtgggtggaggaagctgacaacaatgaaaatcttcaggagatatgcaaagcagaaaccctcgtgcaggcccgaaagagaaagcgaaccagtatcgagaaccgagtgagaggcaacctggagaatttgttcctgcagtgcccgaaacccacactgcagcagatcagccacatcgcccagcagcttgggctcgagaaggatgtggtccgagtgtggttctgtaaccggcgccagaagggcaagcgatcaagcagcgactatgcacaacgagaggattttgaggctgctgggtctcctttctcagggggaccagtgtcctttcctctggccccagggccccattttggtaccccaggctatgggagccctcacttcactgcactgtactcctcggtccctttccctgagggggaagcctttccccctgtctccgtcaccactctgggctctcccatgcattcaaactgaggtgcctgcccttctaggaatgggggacagggggaggggaggagctagggaaagaaaacctggagtttgtgccagggtttttgggattaagttcttcattcactaaggaaggaattgggaacacaaagggtgggggcaggggagtttggggcaactggttggagggaaggtgaagttcaatgatgctcttgattttaatcccacatcatgtatcacttttttcttaaataaagaagcctgggacacagtagatagacacactta

SEQ ID NO: 13 - protein, 479 aa, Homo sapiens
MRQPPGESDMAVSDALLPSFSTFASGPAGREKTLRQAGAPNNRWREELSHMKRLPPVLPGRPYDLAAATVATDLESGGAGAACGGSNLAPLPRRETEEFNDLLDLDFILSNSLTHPPESVAATVSSSASASSSSSPSSSGPASAPSTCSFTYPIRAGNDPGVAPGGTGGGLLYGRESAPPPTAPFNLADINDVSPSGGFVAELLRPELDPVYIPPQQPQPPGGGLMGKFVLKASLSAPGSEYGSPSVISVSKGSPDGSHPVVVAPYNGGPPRTCPKIKQEAVSSCTHLGAGPPLSNGHRPAAHDFPLGRQLPSRTTPTLGLEEVLSSRDCHPALPLPPGFHPHPGPNYPSFLPDQMQPQVPPLHYQELMPPGSCMPEEPKPKRGRRSWPRKRTATHTCDYAGCGKTYTKSSHLKAHLRTHTGEKPYHCDWDGCGWKFARSDELTRHYRKHTGHRPFQCQKCDRAFSRSDHLALHMKRHF

SEQ ID NO: 14 - DNA, 2936 bp, Homo sapiens
  source 1..2936: mol_type = genomic DNA
ggcagtttcccgaccagagagaacgaacgtgtctgcgggcgcgcggggagcagaggcggtggcgggcggcggcggcaccgggagccgccgagtgaccctcccccgcccctctggccccccaccctcccacccgcccgtggcccgcgcccatggccgcgcgcgctccacacaactcaccggagtccgcgccttgcgccgccgaccagttcgcagctccgcgccacggcagccagtctcacctggcggcaccgcccgcccaccgccccggccacagcccctgcgcccacggcagcactcgaggcgaccgcgacagtggtgggggacgctgctgagtggaagagagcgcagcccggccaccggacctacttactcgccttgctgattgtctatttttgcgtttacaacttttctaagaacttttgtatacaaaggaactttttaaaaaagacgcttccaagttatatttaatccaaagaagaaggatctcggccaatttggggttttgggttttggcttcgtttcttctcttcgttgactttggggttcaggtgccccagctgcttcgggctgccgaggaccttctgggcccccacattaatgaggcagccacctggcgagtctgacatggctgtcagcgacgcgctgctcccatctttctccacgttcgcgtctggcccggcgggaagggagaagacactgcgtcaagcaggtgccccgaataaccgctggcgggaggagctctcccacatgaagcgacttcccccagtgcttcccggccgcccctatgacctggcggcggcgaccgtggccacagacctggagagcggcggagccggtgcggcttgcggcggtagcaacctggcgcccctacctcggagagagaccgaggagttcaacgatctcctggacctggactttattctctccaattcgctgacccatcctccggagtcagtggccgccaccgtgtcctcgtcagcgtcagcctcctcttcgtcgtcgccgtcgagcagcggccctgccagcgcgccctccacctgcagcttcacctatccgatccgggccgggaacgacccgggcgtggcgccgggcggcacgggcggaggcctcctctatggcagggagtccgctccccctccgacggctcccttcaacctggcggacatcaacgacgtgagcccctcgggcggcttcgtggccgagctcctgcggccagaattggacccggtgtacattccgccgcagcagccgcagccgccaggtggcgggctgatgggcaagttcgtgctgaaggcgtcgctgagcgcccctggcagcgagtacggcagcccgtcggtcatcagcgtcagcaaaggcagccctgacggcagccacccggtggtggtggcgccctacaacggcgggccgccgcgcacgtgccccaagatcaagcaggaggcggtctcttcgtgcacccacttgggcgctggaccccctctcagcaatggccaccggccggctgcacacgacttccccctggggcggcagctccccagcaggactaccccgaccctgggtcttgaggaagtgctgagcagcagggactgtcaccctgccctgccgcttcctcccggcttccatccccacccggggcccaattacccatccttcctgcccgatcagatgcagccgcaagtcccgccgctccattaccaagagctcatgccacccggttcctgcatgccagaggagcccaagccaaagaggggaagacgatcgtggccccggaaaaggaccgccacccacacttgtgattacgcgggctgcggcaaaacctacacaaagagttcccatctcaaggcacacctgcgaacccacacaggtgagaaaccttaccactgtgactgggacggctgtggatggaaattcgcccgctcagatgaactgaccaggcactaccgtaaacacacggggcaccgcccgttccagtgccaaaaatgcgaccgagcattttccaggtcggaccacctcgccttacacatgaagaggcatttttaaatcccagacagtggatatgacccacactgccagaagagaattcagtattttttacttttcacactgtcttcccgatgagggaaggagcccagccagaaagcactacaatcatggtcaagttcccaactgagtcatcttgtgagtggataatcaggaaaaatgaggaatccaaaagacaaaaatcaaagaacagatggggtctgtgactggatcttctatcattccaattctaaatccgacttgaatattcctggacttacaaaatgccaagggggtgactggaagttgtggatatcagggtataaattatatccgtgagttgggggagggaagaccagaattcccttgaattgtgtattgatgcaatataagcataaaagatcaccttgtattctctttaccttctaaaagccattattatgatgttagaagaagaggaagaaattcaggtacagaaaacatgtttaaatagcctaaatgatggtgcttggtgagtcttggttctaaaggtaccaaacaaggaagccaaagttttcaaactgctgcatactttgacaaggaaaatctatatttgtcttccgatcaacatttatgacctaagtcaggtaatatacctggtttacttctttagcatttttatgcagacagtctgttatgcactgtggtttcagatgtgcaataatttgtacaatggtttattcccaagtatgccttaagcagaacaaatgtgtttttctatatagttccttgccttaataaatatgtaatataaatttaagcaaacgtctattttgtatatttgtaaactacaaagtaaaatgaacattttgtggagtttgtattttgcatactcaaggtgagaattaagttttaaataaacctataatattttatctgaa

SEQ ID NO: 15 - protein, 454 aa, Homo sapiens
MDFFRVVENQQPPATMPLNVSFTNRNYDLDYDSVQPYFYCDEEENFYQQQQQSELQPPAPSEDIWKKFELLPTPPLSPSRRSGLCSPSYVAVTPFSLRGDNDGGGGSFSTADQLEMVTELLGGDMVNQSFICDPDDETFIKNIIIQDCMWSGFSAAAKLVSEKLASYQAARKDSGSPNPARGHSVCSTSSLYLQDLSAAASECIDPSVVFPYPLNDSSSPKSCASQDSSAFSPSSDSLLSSTESSPQGSPEPLVLHEETPPTTSSDSEEEQEDEEEIDVVSVEKRQAPGKRSESGSPSAGGHSKPPHSPLVLKRCHVSTHQHNYAAPPSTRKDYPAAKRVKLDSVRVLRQISNNRKCTSPRSSDTEENVKRRTHNVLERQRRNELKRSFFALRDQIPELENNEKAPKVVILKKATAYILSVQAEEQKLISEEDLLRKRREQLKHKLEQLRNSCA

SEQ ID NO: 16 - DNA, 3721 bp, Homo sapiens
  source 1..3721: mol_type = genomic DNA
aactcgctgtagtaattccagcgagaggcagagggagcgagcgggcggccggctagggtggaagagccgggcgagcagagctgcgctgcgggcgtcctgggaagggagatccggagcgaatagggggcttcgcctctggcccagccctcccgctgatcccccagccagcggtccgcaacccttgccgcatccacgaaactttgcccatagcagcgggcgggcactttgcactggaacttacaacacccgagcaaggacgcgactctcccgacgcggggaggctattctgcccatttggggacacttccccgccgctgccaggacccgcttctctgaaaggctctccttgcagctgcttagacgctggatttttttcgggtagtggaaaaccagcagcctcccgcgacgatgcccctcaacgttagcttcaccaacaggaactatgacctcgactacgactcggtgcagccgtatttctactgcgacgaggaggagaacttctaccagcagcagcagcagagcgagctgcagcccccggcgcccagcgaggatatctggaagaaattcgagctgctgcccaccccgcccctgtcccctagccgccgctccgggctctgctcgccctcctacgttgcggtcacacccttctcccttcggggagacaacgacggcggtggcgggagcttctccacggccgaccagctggagatggtgaccgagctgctgggaggagacatggtgaaccagagtttcatctgcgacccggacgacgagaccttcatcaaaaacatcatcatccaggactgtatgtggagcggcttctcggccgccgccaagctcgtctcagagaagctggcctcctaccaggctgcgcgcaaagacagcggcagcccgaaccccgcccgcggccacagcgtctgctccacctccagcttgtacctgcaggatctgagcgccgccgcctcagagtgcatcgacccctcggtggtcttcccctaccctctcaacgacagcagctcgcccaagtcctgcgcctcgcaagactccagcgccttctctccgtcctcggattctctgctctcctcgacggagtcctccccgcagggcagccccgagcccctggtgctccatgaggagacaccgcccaccaccagcagcgactctgaggaggaacaagaagatgaggaagaaatcgatgttgtttctgtggaaaagaggcaggctcctggcaaaaggtcagagtctggatcaccttctgctggaggccacagcaaacctcctcacagcccactggtcctcaagaggtgccacgtctccacacatcagcacaactacgcagcgcctccctccactcggaaggactatcctgctgccaagagggtcaagttggacagtgtcagagtcctgagacagatcagcaacaaccgaaaatgcaccagccccaggtcctcggacaccgaggagaatgtcaagaggcgaacacacaacgtcttggagcgccagaggaggaacgagctaaaacggagcttttttgccctgcgtgaccagatcccggagttggaaaacaatgaaaaggcccccaaggtagttatccttaaaaaagccacagcatacatcctgtccgtccaagcagaggagcaaaagctcatttctgaagaggacttgttgcggaaacgacgagaacagttgaaacacaaacttgaacagctacggaactcttgtgcgtaaggaaaagtaaggaaaacgattccttctaacagaaatgtcctgagcaatcacctatgaacttgtttcaaatgcatgatcaaatgcaacctcacaaccttggctgagtcttgagactgaaagatttagccataatgtaaactgcctcaaattggactttgggcataaaagaacttttttatgcttaccatcttttttttttctttaacagatttgtatttaagaattgtttttaaaaaattttaagatttacacaatgtttctctgtaaatattgccattaaatgtaaataactttaataaaacgtttatagcagttacacagaatttcaatcctagtatatagtacctagtattataggtactataaaccctaattttttttatttaagtacattttgctttttaaagttgatttttttctattgtttttagaaaaaataaaataactggcaaatatatcattgagccaaatcttaagttgtgaatgttttgtttcgtttcttccccctcccaaccaccaccatccctgtttgttttcatcaattgccccttcagagggtggtcttaagaaaggcaagagttttcctctgttgaaatgggtctgggggccttaaggtctttaagttcttggaggttctaagatgcttcctggagactatgataacagccagagttgacagttagaaggaatggcagaaggcaggtgagaaggtgagaggtaggcaaaggagatacaagaggtcaaaggtagcagttaagtacacaaagaggcataaggactggggagttgggaggaaggtgaggaagaaactcctgttactttagttaaccagtgccagtcccctgctcactccaaacccaggaattctgcccagttgatggggacacggtgggaaccagcttctgctgccttcacaaccaggcgccagtcctgtccatgggttatctcgcaaaccccagaggatctctgggaggaatgctactattaaccctatttcacaaacaaggaaatagaagagctcaaagaggttatgtaacttatctgtagccacgcagataatacaaagcagcaatctggacccattctgttcaaaacacttaacccttcgctatcatgccttggttcatctgggtctaatgtgctgagatcaagaaggtttaggacctaatggacagactcaagtcataacaatgctaagctctatttgtgtcccaagcactcctaagcattttatccctaactctacatcaaccccatgaaggagatactgttgatttccccatattagaagtagagagggaagctgaggcacacaaagactcatccacatgcccaagattcactgatagggaaaagtggaagcgagatttgaacccaggctgtttactcctaacctgtccaagccacctctcagacgacggtaggaatcagctggctgcttgtgagtacaggagttacagtccagtgggttatgttttttaagtctcaacatctaagcctggtcaggcatcagttcccctttttttgtgatttattttgtttttattttgttgttcattgtttaatttttccttttacaatgagaaggtcaccatcttgactcctaccttagccatttgttgaatcagactcatgacggctcctgggaagaagccagttcagatcataaaataaaacatatttattctttgtcatgggagtcattattttagaaactacaaactctccttgcttccatccttttttacatactcatgacacatgctcatcctgagtccttgaaaaggtatttttgaacatgtgtattaattataagcctctgaaaacctatggcccaaaccagaaatgatgttgattatataggtaaatgaaggatgctattgctgttctaattacctcattgtctcagtctcaaagtaggtcttcagctccctgtactttgggattttaatctaccaccacccataaatcaataaataattactttctttga

SEQ ID NO: 17 - DNA, 20 bp, synthetic construct
  misc_feature 1..20: note = Single strand DNA oligonucleotide
  source 1..20: mol_type = other DNA
tggtatcgtggaaggactca

SEQ ID NO: 18 - DNA, 20 bp, synthetic construct
  misc_feature 1..20: note = Single strand DNA oligonucleotide
  source 1..20: mol_type = other DNA
ttcagctcagggatgacctt

SEQ ID NO: 19 - DNA, 19 bp, synthetic construct
  misc_feature 1..19: note = Single strand DNA oligonucleotide
  source 1..19: mol_type = other DNA
agcctgtcctttggaccac

SEQ ID NO: 20 - DNA, 20 bp, synthetic construct
  misc_feature 1..20: note = Single strand DNA oligonucleotide
  source 1..20: mol_type = other DNA
aaccctggagaccagagtcc

SEQ ID NO: 21 - DNA, 20 bp, synthetic construct
  misc_feature 1..20: note = Single strand DNA oligonucleotide
  source 1..20: mol_type = other DNA
gaaagcatctctggctcacc

SEQ ID NO: 22 - DNA, 20 bp, synthetic construct
  misc_feature 1..20: note = Single strand DNA oligonucleotide
  source 1..20: mol_type = other DNA
ctgtctccgtcaccactctg

SEQ ID NO: 23 - DNA, 18 bp, synthetic construct
  misc_feature 1..18: note = Single strand DNA oligonucleotide
  source 1..18: mol_type = other DNA
gcacactgcccctctcac

SEQ ID NO: 24 - DNA, 20 bp, synthetic construct
  misc_feature 1..20: note = Single strand DNA oligonucleotide
  source 1..20: mol_type = other DNA
gaccacctcgccttacacat

SEQ ID NO: 25 - DNA, 20 bp, synthetic construct
  misc_feature 1..20: note = Single strand DNA oligonucleotide
  source 1..20: mol_type = other DNA
agcatacatcctgtccgtcc

SEQ ID NO: 26 - DNA, 22 bp, synthetic construct
  misc_feature 1..22: note = Single strand DNA oligonucleotide
  source 1..22: mol_type = other DNA
agaataccagtcaatctttcac

SEQ ID NO: 27 - DNA, 22 bp, synthetic construct
  misc_feature 1..22: note = Single strand DNA oligonucleotide
  source 1..22: mol_type = other DNA
cctcaacgaccactttgtcaag

SEQ ID NO: 28 - DNA, 22 bp, synthetic construct
  misc_feature 1..22: note = Single strand DNA oligonucleotide
  source 1..22: mol_type = other DNA
tcttcctcttgtgctcttgctg

SEQ ID NO: 29 - DNA, 20 bp, synthetic construct
  misc_feature 1..20: note = Single strand DNA oligonucleotide
  source 1..20: mol_type = other DNA
tcattaagcccaagcgaagg

SEQ ID NO: 30 - DNA, 19 bp, synthetic construct
  misc_feature 1..19: note = Single strand DNA oligonucleotide
  source 1..19: mol_type = other DNA
gtccccattggcattcctc

SEQ ID NO: 31 - DNA, 20 bp, synthetic construct
  misc_feature 1..20: note = Single strand DNA oligonucleotide
  source 1..20: mol_type = other DNA
gagaaggagaagctggagca

SEQ ID NO: 32 - DNA, 20 bp, synthetic construct
  misc_feature 1..20: note = Single strand DNA oligonucleotide
  source 1..20: mol_type = other DNA
cttctgcttcaggagcttgg

SEQ ID NO: 33 - DNA, 20 bp, synthetic construct
  misc_feature 1..20: note = Single strand DNA oligonucleotide
  source 1..20: mol_type = other DNA
agggagaagacactgcgtca

SEQ ID NO: 34 - DNA, 20 bp, synthetic construct
  misc_feature 1..20: note = Single strand DNA oligonucleotide
  source 1..20: mol_type = other DNA
agtcgcttcatgtgggagag

SEQ ID NO: 35 - DNA, 20 bp, synthetic construct
  misc_feature 1..20: note = Single strand DNA oligonucleotide
  source 1..20: mol_type = other DNA
agcgactctgaggaggaaca

SEQ ID NO: 36 - DNA, 20 bp, synthetic construct
  misc_feature 1..20: note = Single strand DNA oligonucleotide
  source 1..20: mol_type = other DNA
ctctgaccttttgccaggag

SEQ ID NO: 37 - DNA, 22 bp, synthetic construct
  misc_feature 1..22: note = Single strand DNA oligonucleotide
  source 1..22: mol_type = other DNA
acgacccctccaagataatttt

SEQ ID NO: 38 - DNA, 20 bp, synthetic construct
  misc_feature 1..20: note = Single strand DNA oligonucleotide
  source 1..20: mol_type = other DNA
gtcgggggtcgttgaatgat

SEQ ID NO: 39 - DNA, 18 bp, synthetic construct
  misc_feature 1..18: note = Single strand DNA oligonucleotide
  source 1..18: mol_type = other DNA
ggttgaatcttccggccg

SEQ ID NO: 40 - DNA, 21 bp, synthetic construct
  misc_feature 1..21: note = Single strand DNA oligonucleotide
  source 1..21: mol_type = other DNA
tctgccactggtttactagga

SEQ ID NO: 41 - DNA, 23 bp, synthetic construct
  misc_feature 1..23: note = Single strand DNA oligonucleotide
  source 1..23: mol_type = other DNA
ggaccacctggtattctgtattt

SEQ ID NO: 42 - DNA, 21 bp, synthetic construct
  misc_feature 1..21: note = Single strand DNA oligonucleotide
  source 1..21: mol_type = other DNA
ctgggcaacaaaggactatga

SEQ ID NO: 43 - DNA, 20 bp, synthetic construct
  misc_feature 1..20: note = Single strand DNA oligonucleotide
  source 1..20: mol_type = other DNA
gaaccgaccactcatcaagc

SEQ ID NO: 44 - DNA, 20 bp, synthetic construct
  misc_feature 1..20: note = Single strand DNA oligonucleotide
  source 1..20: mol_type = other DNA
ttcttcatggtcagtggcct

SEQ ID NO: 45 - DNA, 22 bp, synthetic construct
  misc_feature 1..22: note = Single strand DNA oligonucleotide
  source 1..22: mol_type = other DNA
cagcatcctatcacctcctggt

SEQ ID NO: 46 - DNA, 22 bp, synthetic construct
  misc_feature 1..22: note = Single strand DNA oligonucleotide
  source 1..22: mol_type = other DNA
ctggaacatctccatccttggt

SEQ ID NO: 47 - DNA, 19 bp, synthetic construct
  misc_feature 1..19: note = Single strand DNA oligonucleotide
  source 1..19: mol_type = other DNA
aagaaccagcgtgccaagt

SEQ ID NO: 48 - DNA, 18 bp, synthetic construct
  misc_feature 1..18: note = Single strand DNA oligonucleotide
  source 1..18: mol_type = other DNA
tccagctcctcctgcttg

SEQ ID NO: 49 - DNA, 22 bp, synthetic construct
  misc_feature 1..22: note = Single strand DNA oligonucleotide
  source 1..22: mol_type = other DNA
agaaacgaagatccccagatga

SEQ ID NO: 50 - DNA, 22 bp, synthetic construct
  misc_feature 1..22: note = Single strand DNA oligonucleotide
  source 1..22: mol_type = other DNA
ctgttgctgttgcctgtacgtt

SEQ ID NO: 51 - DNA, 20 bp, synthetic construct
  misc_feature 1..20: note = Single strand DNA oligonucleotide
  source 1..20: mol_type = other DNA
gctcccactccatgaggtat

SEQ ID NO: 52 - DNA, 20 bp, synthetic construct
  misc_feature 1..20: note = Single strand DNA oligonucleotide
  source 1..20: mol_type = other DNA
agtctgtgactgggccttca

SEQ ID NO: 53 - DNA, 20 bp, synthetic construct
  misc_feature 1..20: note = Single strand DNA oligonucleotide
  source 1..20: mol_type = other DNA
agccaacaacattgacacca

SEQ ID NO: 54 - DNA, 20 bp, synthetic construct
  misc_feature 1..20: note = Single strand DNA oligonucleotide
  source 1..20: mol_type = other DNA
tttgaaggactacggctgct

SEQ ID NO: 55 - DNA, 20 bp, synthetic construct
  misc_feature 1..20: note = Single strand DNA oligonucleotide
  source 1..20: mol_type = other DNA
ttgaatggtcaatgcgagtg

SEQ ID NO: 56 - DNA, 20 bp, synthetic construct
  misc_feature 1..20: note = Single strand DNA oligonucleotide
  source 1..20: mol_type = other DNA
cgcagagggttgtattggtt

SEQ ID NO: 57 - DNA, 19 bp, synthetic construct
  misc_feature 1..19: note = Single strand DNA oligonucleotide
  source 1..19: mol_type = other DNA
actgggcagatcctcaagc

SEQ ID NO: 58 - DNA, 20 bp, synthetic construct
  misc_feature 1..20: note = Single strand DNA oligonucleotide
  source 1..20: mol_type = other DNA
gtcatggttgtgcgagtttg

SEQ ID NO: 59 - DNA, 20 bp, synthetic construct
  misc_feature 1..20: note = Single strand DNA oligonucleotide
  source 1..20: mol_type = other DNA
ctaacccaccggcacagtat

SEQ ID NO: 60 - DNA, 20 bp, synthetic construct
  misc_feature 1..20: note = Single strand DNA oligonucleotide
  source 1..20: mol_type = other DNA
tcgactgtcatggatttgga

SEQ ID NO: 61 - DNA, 24 bp, synthetic construct
  misc_feature 1..24: note = Single strand DNA oligonucleotide
  source 1..24: mol_type = other DNA
ttgggaagaggagacacggaacac

SEQ ID NO: 62 - DNA, 24 bp, synthetic construct
  misc_feature 1..24: note = Single strand DNA oligonucleotide
  source 1..24: mol_type = other DNA
ctcctttgttcagccacattggcc

SEQ ID NO: 63 - DNA, 22 bp, synthetic construct
  misc_feature 1..22: note = Single strand DNA oligonucleotide
  source 1..22: mol_type = other DNA
tggcacccatttacacctacac

SEQ ID NO: 64 - DNA, 22 bp, synthetic construct
  misc_feature 1..22: note = Single strand DNA oligonucleotide
  source 1..22: mol_type = other DNA
atgtcaggagaggccccataga

SEQ ID NO: 65 - DNA, 20 bp, synthetic construct
  misc_feature 1..20: note = Single strand DNA oligonucleotide
  source 1..20: mol_type = other DNA
ctgctgcaccttgagtcaga

SEQ ID NO: 66 - DNA, 20 bp, synthetic construct
  misc_feature 1..20: note = Single strand DNA oligonucleotide
  source 1..20: mol_type = other DNA
atgttcagcagggcctcata

SEQ ID NO: 67 - DNA, 20 bp, synthetic construct
  misc_feature 1..20: note = Single strand DNA oligonucleotide
  source 1..20: mol_type = other DNA
ctcgacacccgattcaaagt

SEQ ID NO: 68 - DNA, 20 bp, synthetic construct
  misc_feature 1..20: note = Single strand DNA oligonucleotide
  source 1..20: mol_type = other DNA
ggcgtagaccaagaaatgga

SEQ ID NO: 69 - DNA, 20 bp, synthetic construct
  misc_feature 1..20: note = Single strand DNA oligonucleotide
  source 1..20: mol_type = other DNA
acaaatggacctctcctcca

SEQ ID NO: 70 - DNA, 21 bp, synthetic construct
  misc_feature 1..21: note = Single strand DNA oligonucleotide
  source 1..21: mol_type = other DNA
atggcaatgcacatcacaata

SEQ ID NO: 71 - DNA, 21 bp, synthetic construct
  misc_feature 1..21: note = Single strand DNA oligonucleotide
  source 1..21: mol_type = other DNA
gcagctcaggaagaatgtgtc

SEQ ID NO: 72 - DNA, 21 bp, synthetic construct
  misc_feature 1..21: note = Single strand DNA oligonucleotide
  source 1..21: mol_type = other DNA
tgaagtacactggcattgacg

SEQ ID NO: 73 - DNA, 20 bp, synthetic construct
  misc_feature 1..20: note = Single strand DNA oligonucleotide
  source 1..20: mol_type = other DNA
ccagaacgtcacagtgctca

SEQ ID NO: 74 - DNA, 19 bp, synthetic construct
  misc_feature 1..19: note = Single strand DNA oligonucleotide
  source 1..19: mol_type = other DNA
aggtgttctgagccagcag

SEQ ID NO: 75 - DNA, 20 bp, synthetic construct
  misc_feature 1..20: note = Single strand DNA oligonucleotide
  source 1..20: mol_type = other DNA
ttttcccattctggctccta

SEQ ID NO: 76 - DNA, 20 bp, synthetic construct
  misc_feature 1..20: note = Single strand DNA oligonucleotide
  source 1..20: mol_type = other DNA
tggtgatgctgaaagagacg

SEQ ID NO: 77 - DNA, 23 bp, synthetic construct
  misc_feature 1..23: note = Single strand DNA oligonucleotide
  source 1..23: mol_type = other DNA
ccgacactcctacaagatttaga

SEQ ID NO: 78 - DNA, 23 bp, synthetic construct
  misc_feature 1..23: note = Single strand DNA oligonucleotide
  source 1..23: mol_type = other DNA
caaagatttattgaagcagaacc

SEQ ID NO: 79 - DNA, 20 bp, synthetic construct
  misc_feature 1..20: note = Single strand DNA oligonucleotide
  source 1..20: mol_type = other DNA
gtgacgaagcacagagcaaa

SEQ ID NO: 80 - DNA, 20 bp, synthetic construct
  misc_feature 1..20: note = Single strand DNA oligonucleotide
  source 1..20: mol_type = other DNA
tggtgatgatgccatgttct

SEQ ID NO: 81 - protein, 366 aa, Homo sapiens
MEKYLTPQLPPVPIIPEHKKYRRDSASVVDQFFTDTEGLPYSINMNVFLPDITHLRTGLYKSQRPCVTHIKTEPVAIFSHQSETTAPPPAPTQALPEFTSIFSSHQTAAPEVNNIFIKQELPTPDLHLSVPTQQGHLYQLLNTPDLDMPSSTNQTAAMDTLNVSMSAAMAGLNTHTSAVPQTAVKQFQGMPPCTYTMPSQFLPQQATYFPPSPPSSEPGSPDRQAEMLQNLTPPPSYAATIASKLAIHNPNLPTTLPVNSQNIQPVRYNRRSNPDLEKRRIHYCDYPGCTKVYTKSSHLKAHLRTHTGEKPYKCTWEGCDWRFARSDELTRHYRKHTGAKPFQCGVCNRSFSRSDHLALHMKRHQN

SEQ ID NO: 82 - protein, 457 aa, Homo sapiens
MATRVLSMSARLGPVPQPPAPQDEPVFAQLKPVLGAANPARDAALFPGEELKHAHHRPQAQPAPAQAPQPAQPPATGPRLPPEDLVQTRCEMEKYLTPQLPPVPIIPEHKKYRRDSASVVDQFFTDTEGLPYSINMNVFLPDITHLRTGLYKSQRPCVTHIKTEPVAIFSHQSETTAPPPAPTQALPEFTSIFSSHQTAAPEVNNIFIKQELPTPDLHLSVPTQQGHLYQLLNTPDLDMPSSTNQTAAMDTLNVSMSAAMAGLNTHTSAVPQTAVKQFQGMPPCTYTMPSQFLPQQATYFPPSPPSSEPGSPDRQAEMLQNLTPPPSYAATIASKLAIHNPNLPTTLPVNSQNIQPVRYNRRSNPDLEKRRIHYCDYPGCTKVYTKSSHLKAHLRTHTGEKPYKCTWEGCDWRFARSDELTRHYRKHTGAKPFQCGVCNRSFSRSDHLALHMKRHQN

SEQ ID NO: 83 - DNA, 3349 bp, Homo sapiens
  source 1..3349: mol_type = genomic DNA
agtcgcggggcaggtacgtgcgctcgcggttctctcgcggaggtcggcggtggcgggagcgggctccggagagcctgagagcacggtggggcggggcgggagaaagtggccgcccggaggacgttggcgtttacgtgtggaagagcggaagagttttgcttttcgtgcgcgccttcgaaaactgcctgccgctgtctgaggagtccacccgaaacctcccctcctccgccggcagccccgcgctgagctcgccgacccaagccagcgtgggcgaggtgggaagtgcgcccgacccgcgcctggagctgcgcccccgagtgcccatggctacaagggtgctgagcatgagcgcccgcctgggacccgtgccccagccgccggcgccgcaggacgagccggtgttcgcgcagctcaagccggtgctgggcgccgcgaatccggcccgcgacgcggcgctcttccccggcgaggagctgaagcacgcgcaccaccgcccgcaggcgcagcccgcgcccgcgcaggccccgcagccggcccagccgcccgccaccggcccgcggctgcctccagaggacctggtccagacaagatgtgaaatggagaagtatctgacacctcagcttcctccagttcctataattccagagcataaaaagtatagacgagacagtgcctcagtcgtagaccagttcttcactgacactgaagggttaccttacagtatcaacatgaacgtcttcctccctgacatcactcacctgagaactggcctctacaaatcccagagaccgtgcgtaacacacatcaagacagaacctgttgccattttcagccaccagagtgaaacgactgcccctcctccggccccgacccaggccctccctgagttcaccagtatattcagctcacaccagaccgcagctccagaggtgaacaatattttcatcaaacaagaacttcctacaccagatcttcatctttctgtccctacccagcagggccacctgtaccagctactgaatacaccggatctagatatgcccagttctacaaatcagacagcagcaatggacactcttaatgtttctatgtcagctgccatggcaggccttaacacacacacctctgctgttccgcagactgcagtgaaacaattccagggcatgcccccttgcacatacacaatgccaagtcagtttcttccacaacaggccacttactttcccccgtcaccaccaagctcagagcctggaagtccagatagacaagcagagatgctccagaatttaaccccacctccatcctatgctgctacaattgcttctaaactggcaattcacaatccaaatttacccaccaccctgccagttaactcacaaaacatccaacctgtcagatacaatagaaggagtaaccccgatttggagaaacgacgcatccactactgcgattaccctggttgcacaaaagtttataccaagtcttctcatttaaaagctcacctgaggactcacactggtgaaaagccatacaagtgtacctgggaaggctgcgactggaggttcgcgcgatcggatgagctgacccgccactaccggaagcacacaggcgccaagcccttccagtgcggggtgtgcaaccgcagcttctcgcgctctgaccacctggccctgcatatgaagaggcaccagaactgagcactgcccgtgtgacccgttccaggtcccctgggctccctcaaatgacagacctaactattcctgtgtaaaaacaacaaaaacaaacaaaagcaagaaaaccacaactaaaactggaaatgtatattttgtatatttgagaaaacagggaatacattgtattaataccaaagtgtttggtcattttaagaatctggaatgcttgctgtaatgtatatggctttactcaagcagatctcatctcatgacaggcagccacgtctcaacatgggtaaggggtgggggtggaggggagtgtgtgcagcgtttttacctaggcaccatcatttaatgtgacagtgttcagtaaacaaatcagttggcaggcaccagaagaagaatggattgtatgtcaagattttacttggcattgagtagtttttttcaatagtaggtaattccttagagatacagtatacctggcaattcacaaatagccattgaacaaatgtgtgggtttttaaaaattatatacatatatgagttgcctatatttgctattcaaaattttgtaaatatgcaaatcagctttataggtttattacaagttttttaggattcttttggggaagagtcataattcttttgaaaataaccatgaatacacttacagttaggatttgtggtaaggtacctctcaacattaccaaaatcatttctttagagggaaggaataatcattcaaatgaactttaaaaaagcaaatttcatgcactgattaaaataggattattttaaatacaaaaggcattttatatgaattataaactgaagagcttaaagatagttacaaaatacaaaagttcaacctcttacaataagctaaacgcaatgtcatttttaaaaagaaggacttagggtgtcgttttcacatatgacaatgttgcatttatgatgcagtttcaagtaccaaaacgttgaattgatgatgcagttttcatatatcgagatgttcgctcgtgcagtactgttggttaaatgacaatttatgtggattttgcatgtaatacacagtgagacacagtaattttatctaaattacagtgcagtttagttaatctattaatactgactcagtgtctgcctttaaatataaatgatatgttgaaaacttaaggaagcaaatgctacatatatgcaatataaaatagtaatgtgatgctgatgctgttaaccaaagggcagaataaataagcaaaatgccaaaaggggtcttaattgaaatgaaaatttaattttgtttttaaaatattgtttatctttatttattttgtggtaatatagtaagtttttttagaagacaattttcataacttgataaattatagttttgtttgttagaaaagttgctcttaaaagatgtaaatagatgacaaacgatgtaaataattttgtaagaggcttcaaaatgtttatacgtggaaacacacctacatgaaaagcagaaatcggttgctgttttgcttctttttccctcttatttttgtattgtggtcatttcctatgcaaataatggagcaaacagctgtatagttgtagaattttttgagagaatgagatgtttatatattaacgacaattttttttttggaaaataaaaagtgcctaaaaga

SEQ ID NO: 84 - DNA, 2948 bp, Homo sapiens
  source 1..2948: mol_type = genomic DNA
actttagctcagacctttcttttaaccttgcctatcatgtttcgagtcagaatttaaatactgtgcagtttaagctacaatacgcttggcctataacttggttccaggcatttatatttatgtcacttttgtctacttattatactaacaaggtggaaaaagcaatcccagtctctccaaaagacaagatgtgaaatggagaagtatctgacacctcagcttcctccagttcctataattccagagcataaaaagtatagacgagacagtgcctcagtcgtagaccagttcttcactgacactgaagggttaccttacagtatcaacatgaacgtcttcctccctgacatcactcacctgagaactggcctctacaaatcccagagaccgtgcgtaacacacatcaagacagaacctgttgccattttcagccaccagagtgaaacgactgcccctcctccggccccgacccaggccctccctgagttcaccagtatattcagctcacaccagaccgcagctccagaggtgaacaatattttcatcaaacaagaacttcctacaccagatcttcatctttctgtccctacccagcagggccacctgtaccagctactgaatacaccggatctagatatgcccagttctacaaatcagacagcagcaatggacactcttaatgtttctatgtcagctgccatggcaggccttaacacacacacctctgctgttccgcagactgcagtgaaacaattccagggcatgcccccttgcacatacacaatgccaagtcagtttcttccacaacaggccacttactttcccccgtcaccaccaagctcagagcctggaagtccagatagacaagcagagatgctccagaatttaaccccacctccatcctatgctgctacaattgcttctaaactggcaattcacaatccaaatttacccaccaccctgccagttaactcacaaaacatccaacctgtcagatacaatagaaggagtaaccccgatttggagaaacgacgcatccactactgcgattaccctggttgcacaaaagtttataccaagtcttctcatttaaaagctcacctgaggactcacactggtgaaaagccatacaagtgtacctgggaaggctgcgactggaggttcgcgcgatcggatgagctgacccgccactaccggaagcacacaggcgccaagcccttccagtgcggggtgtgcaaccgcagcttctcgcgctctgaccacctggccctgcatatgaagaggcaccagaactgagcactgcccgtgtgacccgttccaggtcccctgggctccctcaaatgacagacctaactattcctgtgtaaaaacaacaaaaacaaacaaaagcaagaaaaccacaactaaaactggaaatgtatattttgtatatttgagaaaacagggaatacattgtattaataccaaagtgtttggtcattttaagaatctggaatgcttgctgtaatgtatatggctttactcaagcagatctcatctcatgacaggcagccacgtctcaacatgggtaaggggtgggggtggaggggagtgtgtgcagcgtttttacctaggcaccatcatttaatgtgacagtgttcagtaaacaaatcagttggcaggcaccagaagaagaatggattgtatgtcaagattttacttggcattgagtagtttttttcaatagtaggtaattccttagagatacagtatacctggcaattcacaaatagccattgaacaaatgtgtgggtttttaaaaattatatacatatatgagttgcctatatttgctattcaaaattttgtaaatatgcaaatcagctttataggtttattacaagttttttaggattcttttggggaagagtcataattcttttgaaaataaccatgaatacacttacagttaggatttgtggtaaggtacctctcaacattaccaaaatcatttctttagagggaaggaataatcattcaaatgaactttaaaaaagcaaatttcatgcactgattaaaataggattattttaaatacaaaaggcattttatatgaattataaactgaagagcttaaagatagttacaaaatacaaaagttcaacctcttacaataagctaaacgcaatgtcatttttaaaaagaaggacttagggtgtcgttttcacatatgacaatgttgcatttatgatgcagtttcaagtaccaaaacgttgaattgatgatgcagttttcatatatcgagatgttcgctcgtgcagtactgttggttaaatgacaatttatgtggattttgcatgtaatacacagtgagacacagtaattttatctaaattacagtgcagtttagttaatctattaatactgactcagtgtctgcctttaaatataaatgatatgttgaaaacttaaggaagcaaatgctacatatatgcaatataaaatagtaatgtgatgctgatgctgttaaccaaagggcagaataaataagcaaaatgccaaaaggggtcttaattgaaatgaaaatttaattttgtttttaaaatattgtttatctttatttattttgtggtaatatagtaagtttttttagaagacaattttcataacttgataaattatagttttgtttgttagaaaagttgctcttaaaagatgtaaatagatgacaaacgatgtaaataattttgtaagaggcttcaaaatgtttatacgtggaaacacacctacatgaaaagcagaaatcggttgctgttttgcttctttttccctcttatttttgtattgtggtcatttcctatgcaaataatggagcaaacagctgtatagttgtagaattttttgagagaatgagatgtttatatattaacgacaattttttttttggaaaataaaaagtgcctaaaaga

SEQ ID NO: 85 - protein, 241 aa, Homo sapiens
MDVLPMCSIFQELQIVHETGYFSALPSLEEYWQQTCLELERYLQSEPCYVSASEIKFDSQEDLWTKIILAREKKEESELKISSSPPEDTLISPSFCYNLETNSLNSDVSSESSDSSEELSPTAKFTSDPIGEVLVSSGKLSSSVTSTPPSSPELSREPSQLWGCVPGELPSPGKVRSGTSGKPGEKPYRCSWEGCEWRFARSDELTRHFRKHTGAKPFKCSHCDRCFSRSDHLALHMKRHL

SEQ ID NO: 86 - protein, 237 aa, Homo sapiens
MDVLPMCSIFQELQIVHETGYFSALPSLEEYWQQTCLELERYLQSEPCYVSASEIKFDSQEDLWTKIILAREKKEESELKISSSPPEDTLISPSFCYNLETNSLNSDVSSESSDSSEELSPTAKFTSDPIGEVLVSSGKLSSSVTSTPPSSPELSREPSQLWGCVPGELPSPGKVRSGTSGKPGDKGNGDASPDGRRRVHRCHFNGCRKVYTKSSHLKAHQRTHTGVFPGLTTWPCT

SEQ ID NO: 87 - protein, 283 aa, Homo sapiens
MDVLPMCSIFQELQIVHETGYFSALPSLEEYWQQTCLELERYLQSEPCYVSASEIKFDSQEDLWTKIILAREKKEESELKISSSPPEDTLISPSFCYNLETNSLNSDVSSESSDSSEELSPTAKFTSDPIGEVLVSSGKLSSSVTSTPPSSPELSREPSQLWGCVPGELPSPGKVRSGTSGKPGDKGNGDASPDGRRRVHRCHFNGCRKVYTKSSHLKAHQRTHTGEKPYRCSWEGCEWRFARSDELTRHFRKHTGAKPFKCSHCDRCFSRSDHLALHMKRHL

SEQ ID NO: 88 - DNA, 1708 bp, Homo sapiens
  source 1..1708: mol_type = genomic DNA
gggggaggctggcagcggagctttgaatagggaagttttgcaggggttacgtttgcagtcagtccggtgtttgcaaatattgtgtgggctccgcgcgctgcgggctgcgggagggtccggccgggcgtctctgcgagcctggagtttgcatgaaactttcacctgcgctccggggagactttcggctccggctcccaccgcgcgcctcgccgccctcgcgaccgcgggctccgtccaacccggcccgacatggacgtgctccccatgtgcagcatcttccaggagctccagatcgtgcacgagaccggctacttctcggcgctgccgtctctggaggagtactggcaacagacctgcctagagctggaacgttacctccagagcgagccctgctatgtttcagcctcagaaatcaaatttgacagccaggaagatctgtggaccaaaatcattctggctcgggagaaaaaggaggaatccgaactgaagatatcttccagtcctccagaggacactctcatcagcccgagcttttgttacaacttagagaccaacagcctgaactcagatgtcagcagcgaatcctctgacagctccgaggaactttctcccacggccaagtttacctccgaccccattggcgaagttttggtcagctcgggaaaattgagctcctctgtcacctccacgcctccatcttctccggaactgagcagggaaccttctcaactgtggggttgcgtgcccggggagctgccctcgccagggaaggtgcgcagcgggacttcggggaagccaggtgacaagggaaatggcgatgcctcccccgacggcaggaggagggtgcaccggtgccactttaacggctgcaggaaagtttacaccaaaagctcccacttgaaagcacaccagcggacgcacacaggagaaaagccttacagatgctcatgggaagggtgtgagtggcgttttgcaagaagtgatgagttaaccaggcacttccgaaagcacaccggggccaagccttttaaatgctcccactgtgacaggtgtttttccaggtctgaccacctggccctgcacatgaagaggcacctctgagggagcagagaggtggatcctgtaggctaaaaggcttccaggctgagagccggccgtggaaggagggatgcgtgttccagccaaagcatgccgttctgcaccctacccagttgcctccagggcctctccttggaaggtcttttgagggctaaaaaggtcctgtaagaagcggcatagcacccgtggtgcatgcctgtcctgggagctggagaggatgtcagcgagcctgacattgccctccctgaatgcatcaaatactcttctccaaggactgacaaaaacaaccccccggctgtgggcacagtagggaaccatgccggtttgatcttccattgctcaagccagggaatgcattgcagaggcacctgcacaacgacaaacttgagtgtttccctgccacttctgtgcctccacagcctgctccagttccgcagctagagctgggcctaccttccccagccagccctgccacacacctggctgaggcatgtctgggaggggtggaggaggcagagggactccaggccagagggctgttctcacaacctcagcccactctcagtcaaggagggcagaaaataaaagatgacatcactgccaaaaaaaa

SEQ ID NO: 89 - DNA, 4464 bp, Homo sapiens
  source 1..4464: mol_type = genomic DNA
gcagtcagtccggtgtttgcaaatattgtgtgggctccgcgcgctgcgggctgcgggagggtccggccgggcgtctctgcgagcctggagtttgcatgaaactttcacctgcgctccggggagactttcggctccggctcccaccgcgcgcctcgccgccctcgcgaccgcgggctccgtccaacccggcccgacatggacgtgctccccatgtgcagcatcttccaggagctccagatcgtgcacgagaccggctacttctcggcgctgccgtctctggaggagtactggcaacagacctgcctagagctggaacgttacctccagagcgagccctgctatgtttcagcctcagaaatcaaatttgacagccaggaagatctgtggaccaaaatcattctggctcgggagaaaaaggaggaatccgaactgaagatatcttccagtcctccagaggacactctcatcagcccgagcttttgttacaacttagagaccaacagcctgaactcagatgtcagcagcgaatcctctgacagctccgaggaactttctcccacggccaagtttacctccgaccccattggcgaagttttggtcagctcgggaaaattgagctcctctgtcacctccacgcctccatcttctccggaactgagcagggaaccttctcaactgtggggttgcgtgcccggggagctgccctcgccagggaaggtgcgcagcgggacttcggggaagccaggagaaaagccttacagatgctcatgggaagggtgtgagtggcgttttgcaagaagtgatgagttaaccaggcacttccgaaagcacaccggggccaagccttttaaatgctcccactgtgacaggtgtttttccaggtctgaccacctggccctgcacatgaagaggcacctctgagggagcagagaggtggatcctgtaggctaaaaggcttccaggctgagagccggccgtggaaggagggatgcgtgttccagccaaagcatgccgttctgcaccctacccagttgcctccagggcctctccttggaaggtcttttgagggctaaaaaggtcctgtaagaagcggcatagcacccgtggtgcatggtatgtgggtgaccctggactcgccactggtacccgcccttccgagcggcgcctaagcctttgccgtgagcatgcacactgagaatgctaatggttgggttgattgtatgttgaggatctattactgaccgtatgatgaggccaactttttttccttgtggttagcaagactgcaagagatggaaaaaaagtagtttgaatgttttgtgtgtaaggagtataccatgagatgagatgaccaccaatcatttccttggggggagggggtgtctgcaccttagaaaaaaaaagaaaaatcaaaaaaacaaaaaaacaaaaacaaaaaaagaaggaaaatcttggagggtgggcgtgggaactcaggaccccagagtggcgagtggtgtggggagggagagcctctctcccccttttctgtgtgagaggaactcttagtgtctggtgcagctattaaatgtgcaatgtgtcaagtagcttgttttacacgctacaacatagctcatttgtaacccattgtataagctgtgtatttacaaatataacacaacaatttaacttttccttagaatacaaaaagtcatgcatggtctggggaactatatgcttttccatttttaagtcaggactgcaatactgattccagttaatgagcagctaagatccaatctgtctaatacagtgaccccctagccatccgggcctggcaatatacaattttttttcccctccaagtttgtaacactccccttccagaaaggcattgtgcaacacaggattatttttaaatgattctgaatttgaattaactttttggagaattctgtgatgcccttagaagaaattggacacgtattgagtgtcacaaagctggggctgggaattgctggtctaatgtttcattagacttaagaacctaaaatttttctcagttgggtggataaaaccactaacgcttagaaactgttttctcatgcagctatgtttctcttatttatgccttgaggactaatttctggttttctagctgttaatgcactgttgaccttcataatggtgccttacgcaagcgatcccttctgtgggggtctcatacaggggtgtgggcgatgcatgctttattaaggctcttgtttcacctggcagtgtactgtatcaacgtataatacagaaaaaaaatctctttaaggtcctccttcacaaagacatagagtgaaactccctttacatgtcagtatttgttcaacactttaggcaacttgactgtcagtgttaaaatggaaaacaggaaaatggaaaaatctgaccaattctgccaccttgagactttcatatagaccttgcacaacaattgtatagatcacacaccggctgtatttaatatgtaacattttcacacatattaaagatacagaagtattaaaaaacccccaatgttaatgtatttgcttaaaaggcacaagtttcacatatctgtctagctatctgttggtaatacagaaagtatactacttttttaaaaaagtgggcagaattcttgtgtatgtatatttgtgtgtacagtatgtgtatgtgtgtatatatatatattatatatatagataatatataaatattttttttaaggagaaactagaatgtttagctagaaaattccacagcctgtgaagaaatatttcaaaatggccataaaggaggtaaaaatgaaaaccataacctaacttttatagaggctttatctttaatttaacgatgtgcggaggactttcttgcttgaatctgttccgggctgtctgctctgtccatcaaatgggcaggtctggaatggggcaccttcggccgttcagaagtggcctgaacagaatgctggaacccaggctggactcggacacactaaggttttgattttgaatttcagccttattagaagatctaacctaagagtaagctaaccacagggattcttttgtagaacactttttatgcagatgaagctattttttccagcaagtagattcttccagtttttccaaggagtaatttccccgaattggcataccacggcgtggacagctgatatttcacccagctgctggcttgtgggtgtggctctttgctttatatatatatacacacatgtgagtctggctgggctggtattttgtttgatcttcctggaaatgagcagtgactaacgctcacataactggttttttttttatctgggctgatgaatacatttacctaagaaactcatttcgttttacttaagaggggaagtgcagttttcttttggcagttcagaatccaagcacttgatttgctgggtttggaaaactccttttttggccttctatgtgcttagccataacaattccattaagcaagaaggtaagcaaaagacaaaaaaaaaaaaaggaaaaaaaaaaacttgcactggcttgtctcacttacgaaacatgtcggagctgtttgcctgggtggggctgggtaccgtacctgtcaatgcctgtgattttcataattagcacgtacataaagaagtacattctgttcaggtgataactgagcctcaatcaagcagaaactttttgcttgaaattaaaaaaaaatttctattagtgaaatttcttttttttttttttttggaagcaccctgttatctaaagaatctttgtaagatttttgtaaaattttgttttacaagattttatttgaaattgttttttgcaagattgttatatttctgtatgaatgtattttttattggaataacataaaagaattcttatcagcatcttgagtctggttgttttttttgggggaagggggttgttggaacagattcctcctgcattcatcaccctggcttcctcctgggggcaaatcttcattgagcaaccctgagaacaactcaccgacctcagccccttcctctttccacagcctgtcctgggagctggagaggatgtcagcgagcctgacattgccctccctgaatgcatcaaatactcttctccaaggactgacaaaaacaaccccccggctgtgggcacagtagggaaccatgccggtttgatcttccattgctcaagccagggaatgcattgcagaggcacctgcacaacgacaaacttgagtgtttccctgccacttctgtgcctccacagcctgctccagttccgcagctagagctgggcctaccttccccagccagccctgccacacacctggctgaggcatgtctgggaggggtggaggaggcagagggactccaggccagagggctgttctcacaacctcagcccactctcagtcaaggagggcagaaaataaaagatgacatcactgcca

SEQ ID NO: 90 - DNA, 4466 bp, Homo sapiens
  source 1..4466: mol_type = genomic DNA
gcagtcagtccggtgtttgcaaatattgtgtgggctccgcgcgctgcgggctgcgggagggtccggccgggcgtctctgcgagcctggagtttgcatgaaactttcacctgcgctccggggagactttcggctccggctcccaccgcgcgcctcgccgccctcgcgaccgcgggctccgtccaacccggcccgacatggacgtgctccccatgtgcagcatcttccaggagctccagatcgtgcacgagaccggctacttctcggcgctgccgtctctggaggagtactggcaacagacctgcctagagctggaacgttacctccagagcgagccctgctatgtttcagcctcagaaatcaaatttgacagccaggaagatctgtggaccaaaatcattctggctcgggagaaaaaggaggaatccgaactgaagatatcttccagtcctccagaggacactctcatcagcccgagcttttgttacaacttagagaccaacagcctgaactcagatgtcagcagcgaatcctctgacagctccgaggaactttctcccacggccaagtttacctccgaccccattggcgaagttttggtcagctcgggaaaattgagctcctctgtcacctccacgcctccatcttctccggaactgagcagggaaccttctcaactgtggggttgcgtgcccggggagctgccctcgccagggaaggtgcgcagcgggacttcggggaagccaggtgacaagggaaatggcgatgcctcccccgacggcaggaggagggtgcaccggtgccactttaacggctgcaggaaagtttacaccaaaagctcccacttgaaagcacaccagcggacgcacacaggtgtttttccaggtctgaccacctggccctgcacatgaagaggcacctctgagggagcagagaggtggatcctgtaggctaaaaggcttccaggctgagagccggccgtggaaggagggatgcgtgttccagccaaagcatgccgttctgcaccctacccagttgcctccagggcctctccttggaaggtcttttgagggctaaaaaggtcctgtaagaagcggcatagcacccgtggtgcatggtatgtgggtgaccctggactcgccactggtacccgcccttccgagcggcgcctaagcctttgccgtgagcatgcacactgagaatgctaatggttgggttgattgtatgttgaggatctattactgaccgtatgatgaggccaactttttttccttgtggttagcaagactgcaagagatggaaaaaaagtagtttgaatgttttgtgtgtaaggagtataccatgagatgagatgaccaccaatcatttccttggggggagggggtgtctgcaccttagaaaaaaaaagaaaaatcaaaaaaacaaaaaaacaaaaacaaaaaaagaaggaaaatcttggagggtgggcgtgggaactcaggaccccagagtggcgagtggtgtggggagggagagcctctctcccccttttctgtgtgagaggaactcttagtgtctggtgcagctattaaatgtgcaatgtgtcaagtagcttgttttacacgctacaacatagctcatttgtaacccattgtataagctgtgtatttacaaatataacacaacaatttaacttttccttagaatacaaaaagtcatgcatggtctggggaactatatgcttttccatttttaagtcaggactgcaatactgattccagttaatgagcagctaagatccaatctgtctaatacagtgaccccctagccatccgggcctggcaatatacaattttttttcccctccaagtttgtaacactccccttccagaaaggcattgtgcaacacaggattatttttaaatgattctgaatttgaattaactttttggagaattctgtgatgcccttagaagaaattggacacgtattgagtgtcacaaagctggggctgggaattgctggtctaatgtttcattagacttaagaacctaaaatttttctcagttgggtggataaaaccactaacgcttagaaactgttttctcatgcagctatgtttctcttatttatgccttgaggactaatttctggttttctagctgttaatgcactgttgaccttcataatggtgccttacgcaagcgatcccttctgtgggggtctcatacaggggtgtgggcgatgcatgctttattaaggctcttgtttcacctggcagtgtactgtatcaacgtataatacagaaaaaaaatctctttaaggtcctccttcacaaagacatagagtgaaactccctttacatgtcagtatttgttcaacactttaggcaacttgactgtcagtgttaaaatggaaaacaggaaaatggaaaaatctgaccaattctgccaccttgagactttcatatagaccttgcacaacaattgtatagatcacacaccggctgtatttaatatgtaacattttcacacatattaaagatacagaagtattaaaaaacccccaatgttaatgtatttgcttaaaaggcacaagtttcacatatctgtctagctatctgttggtaatacagaaagtatactacttttttaaaaaagtgggcagaattcttgtgtatgtatatttgtgtgtacagtatgtgtatgtgtgtatatatatatattatatatatagataatatataaatattttttttaaggagaaactagaatgtttagctagaaaattccacagcctgtgaagaaatatttcaaaatggccataaaggaggtaaaaatgaaaaccataacctaacttttatagaggctttatctttaatttaacgatgtgcggaggactttcttgcttgaatctgttccgggctgtctgctctgtccatcaaatgggcaggtctggaatggggcaccttcggccgttcagaagtggcctgaacagaatgctggaacccaggctggactcggacacactaaggttttgattttgaatttcagccttattagaagatctaacctaagagtaagctaaccacagggattcttttgtagaacactttttatgcagatgaagctattttttccagcaagtagattcttccagtttttccaaggagtaatttccccgaattggcataccacggcgtggacagctgatatttcacccagctgctggcttgtgggtgtggctctttgctttatatatatatacacacatgtgagtctggctgggctggtattttgtttgatcttcctggaaatgagcagtgactaacgctcacataactggttttttttttatctgggctgatgaatacatttacctaagaaactcatttcgttttacttaagaggggaagtgcagttttcttttggcagttcagaatccaagcacttgatttgctgggtttggaaaactccttttttggccttctatgtgcttagccataacaattccattaagcaagaaggtaagcaaaagacaaaaaaaaaaaaaggaaaaaaaaaaacttgcactggcttgtctcacttacgaaacatgtcggagctgtttgcctgggtggggctgggtaccgtacctgtcaatgcctgtgattttcataattagcacgtacataaagaagtacattctgttcaggtgataactgagcctcaatcaagcagaaactttttgcttgaaattaaaaaaaaatttctattagtgaaatttcttttttttttttttttggaagcaccctgttatctaaagaatctttgtaagatttttgtaaaattttgttttacaagattttatttgaaattgttttttgcaagattgttatatttctgtatgaatgtattttttattggaataacataaaagaattcttatcagcatcttgagtctggttgttttttttgggggaagggggttgttggaacagattcctcctgcattcatcaccctggcttcctcctgggggcaaatcttcattgagcaaccctgagaacaactcaccgacctcagccccttcctctttccacagcctgtcctgggagctggagaggatgtcagcgagcctgacattgccctccctgaatgcatcaaatactcttctccaaggactgacaaaaacaaccccccggctgtgggcacagtagggaaccatgccggtttgatcttccattgctcaagccagggaatgcattgcagaggcacctgcacaacgacaaacttgagtgtttccctgccacttctgtgcctccacagcctgctccagttccgcagctagagctgggcctaccttccccagccagccctgccacacacctggctgaggcatgtctgggaggggtggaggaggcagagggactccaggccagagggctgttctcacaacctcagcccactctcagtcaaggagggcagaaaataaaagatgacatcactgcca

SEQ ID NO: 91 - DNA, 4590 bp, Homo sapiens
  source 1..4590: mol_type = genomic DNA
gcagtcagtccggtgtttgcaaatattgtgtgggctccgcgcgctgcgggctgcgggagggtccggccgggcgtctctgcgagcctggagtttgcatgaaactttcacctgcgctccggggagactttcggctccggctcccaccgcgcgcctcgccgccctcgcgaccgcgggctccgtccaacccggcccgacatggacgtgctccccatgtgcagcatcttccaggagctccagatcgtgcacgagaccggctacttctcggcgctgccgtctctggaggagtactggcaacagacctgcctagagctggaacgttacctccagagcgagccctgctatgtttcagcctcagaaatcaaatttgacagccaggaagatctgtggaccaaaatcattctggctcgggagaaaaaggaggaatccgaactgaagatatcttccagtcctccagaggacactctcatcagcccgagcttttgttacaacttagagaccaacagcctgaactcagatgtcagcagcgaatcctctgacagctccgaggaactttctcccacggccaagtttacctccgaccccattggcgaagttttggtcagctcgggaaaattgagctcctctgtcacctccacgcctccatcttctccggaactgagcagggaaccttctcaactgtggggttgcgtgcccggggagctgccctcgccagggaaggtgcgcagcgggacttcggggaagccaggtgacaagggaaatggcgatgcctcccccgacggcaggaggagggtgcaccggtgccactttaacggctgcaggaaagtttacaccaaaagctcccacttgaaagcacaccagcggacgcacacaggagaaaagccttacagatgctcatgggaagggtgtgagtggcgttttgcaagaagtgatgagttaaccaggcacttccgaaagcacaccggggccaagccttttaaatgctcccactgtgacaggtgtttttccaggtctgaccacctggccctgcacatgaagaggcacctctgagggagcagagaggtggatcctgtaggctaaaaggcttccaggctgagagccggccgtggaaggagggatgcgtgttccagccaaagcatgccgttctgcaccctacccagttgcctccagggcctctccttggaaggtcttttgagggctaaaaaggtcctgtaagaagcggcatagcacccgtggtgcatggtatgtgggtgaccctggactcgccactggtacccgcccttccgagcggcgcctaagcctttgccgtgagcatgcacactgagaatgctaatggttgggttgattgtatgttgaggatctattactgaccgtatgatgaggccaactttttttccttgtggttagcaagactgcaagagatggaaaaaaagtagtttgaatgttttgtgtgtaaggagtataccatgagatgagatgaccaccaatcatttccttggggggagggggtgtctgcaccttagaaaaaaaaagaaaaatcaaaaaaacaaaaaaacaaaaacaaaaaaagaaggaaaatcttggagggtgggcgtgggaactcaggaccccagagtggcgagtggtgtggggagggagagcctctctcccccttttctgtgtgagaggaactcttagtgtctggtgcagctattaaatgtgcaatgtgtcaagtagcttgttttacacgctacaacatagctcatttgtaacccattgtataagctgtgtatttacaaatataacacaacaatttaacttttccttagaatacaaaaagtcatgcatggtctggggaactatatgcttttccatttttaagtcaggactgcaatactgattccagttaatgagcagctaagatccaatctgtctaatacagtgaccccctagccatccgggcctggcaatatacaattttttttcccctccaagtttgtaacactccccttccagaaaggcattgtgcaacacaggattatttttaaatgattctgaatttgaattaactttttggagaattctgtgatgcccttagaagaaattggacacgtattgagtgtcacaaagctggggctgggaattgctggtctaatgtttcattagacttaagaacctaaaatttttctcagttgggtggataaaaccactaacgcttagaaactgttttctcatgcagctatgtttctcttatttatgccttgaggactaatttctggttttctagctgttaatgcactgttgaccttcataatggtgccttacgcaagcgatcccttctgtgggggtctcatacaggggtgtgggcgatgcatgctttattaaggctcttgtttcacctggcagtgtactgtatcaacgtataatacagaaaaaaaatctctttaaggtcctccttcacaaagacatagagtgaaactccctttacatgtcagtatttgttcaacactttaggcaacttgactgtcagtgttaaaatggaaaacaggaaaatggaaaaatctgaccaattctgccaccttgagactttcatatagaccttgcacaacaattgtatagatcacacaccggctgtatttaatatgtaacattttcacacatattaaagatacagaagtattaaaaaacccccaatgttaatgtatttgcttaaaaggcacaagtttcacatatctgtctagctatctgttggtaatacagaaagtatactacttttttaaaaaagtgggcagaattcttgtgtatgtatatttgtgtgtacagtatgtgtatgtgtgtatatatatatattatatatatagataatatataaatattttttttaaggagaaactagaatgtttagctagaaaattccacagcctgtgaagaaatatttcaaaatggccataaaggaggtaaaaatgaaaaccataacctaacttttatagaggctttatctttaatttaacgatgtgcggaggactttcttgcttgaatctgttccgggctgtctgctctgtccatcaaatgggcaggtctggaatggggcaccttcggccgttcagaagtggcctgaacagaatgctggaacccaggctggactcggacacactaaggttttgattttgaatttcagccttattagaagatctaacctaagagtaagctaaccacagggattcttttgtagaacactttttatgcagatgaagctattttttccagcaagtagattcttccagtttttccaaggagtaatttccccgaattggcataccacggcgtggacagctgatatttcacccagctgctggcttgtgggtgtggctctttgctttatatatatatacacacatgtgagtctggctgggctggtattttgtttgatcttcctggaaatgagcagtgactaacgctcacataactggttttttttttatctgggctgatgaatacatttacctaagaaactcatttcgttttacttaagaggggaagtgcagttttcttttggcagttcagaatccaagcacttgatttgctgggtttggaaaactccttttttggccttctatgtgcttagccataacaattccattaagcaagaaggtaagcaaaagacaaaaaaaaaaaaaggaaaaaaaaaaacttgcactggcttgtctcacttacgaaacatgtcggagctgtttgcctgggtggggctgggtaccgtacctgtcaatgcctgtgattttcataattagcacgtacataaagaagtacattctgttcaggtgataactgagcctcaatcaagcagaaactttttgcttgaaattaaaaaaaaatttctattagtgaaatttcttttttttttttttttggaagcaccctgttatctaaagaatctttgtaagatttttgtaaaattttgttttacaagattttatttgaaattgttttttgcaagattgttatatttctgtatgaatgtattttttattggaataacataaaagaattcttatcagcatcttgagtctggttgttttttttgggggaagggggttgttggaacagattcctcctgcattcatcaccctggcttcctcctgggggcaaatcttcattgagcaaccctgagaacaactcaccgacctcagccccttcctctttccacagcctgtcctgggagctggagaggatgtcagcgagcctgacattgccctccctgaatgcatcaaatactcttctccaaggactgacaaaaacaaccccccggctgtgggcacagtagggaaccatgccggtttgatcttccattgctcaagccagggaatgcattgcagaggcacctgcacaacgacaaacttgagtgtttccctgccacttctgtgcctccacagcctgctccagttccgcagctagagctgggcctaccttccccagccagccctgccacacacctggctgaggcatgtctgggaggggtggaggaggcagagggactccaggccagagggctgttctcacaacctcagcccactctcagtcaaggagggcagaaaataaaagatgacatcactgcca

SEQ ID NO: 92 - protein, 416 aa, Homo sapiens
MVDHLLPVDENFSSPKCPVGYLGDRLVGRRAYHMLPSPVSEDDSDASSPCSCSSPDSQALCSCYGGGLGTESQDSILDFLLSQATLGSGGGSGSSIGASSGPVAWGPWRRAAAPVKGEHFCLPEFPLGDPDDVPRPFQPTLEEIEEFLEENMEPGVKEVPEGNSKDLDACSQLSAGPHKSHLHPGSSGRERCSPPPGGASAGGAQGPGGGPTPDGPIPVLLQIQPVPVKQESGTGPASPGQAPENVKVAQLLVNIQGQTFALVPQVVPSSNLNLPSKFVRIAPVPIAAKPVGSGPLGPGPAGLLMGQKFPKNPAAELIKMHKCTFPGCSKMYTKSSHLKAHLRRHTGEKPFACTWPGCGWRFSRSDELSRHRRSHSGVKPYQCPVCEKKFARSDHLSKHIKVHRFPRSSRSVRSVN

SEQ ID NO: 93 - DNA, 2540 bp, Homo sapiens
  source 1..2540: mol_type = genomic DNA
agtgctccgccgcagcgacccgcgggccggcgggcgatcgagccagcgcaggacccgcggctcggcccccggccgccgccggaccgagagtctagccgccgcccccagcccagcccgcccggccgcaggaccgccggggcctggccgccggtccggcgtgcgccaagttcagccgccaccggcacggccaggccagcatggtggaccacttacttccagtggacgagaacttctcgtcgccaaaatgcccagttgggtatctgggtgataggctggttggccggcgggcatatcacatgctgccctcacccgtctctgaagatgacagcgatgcctccagcccctgctcctgttccagtcccgactctcaagccctctgctcctgctatggtggaggcctgggcaccgagagccaggacagcatcttggacttcctattgtcccaggccacgctgggcagtggcgggggcagcggcagtagcattggggccagcagtggccccgtggcctgggggccctggcgaagggcagcggcccctgtgaagggggagcatttctgcttgcccgagtttcctttgggtgatcctgatgacgtcccacggcccttccagcctaccctggaggagattgaagagtttctggaggagaacatggagcctggagtcaaggaggtccctgagggcaacagcaaggacttggatgcctgcagccagctctcagctgggccacacaagagccacctccatcctgggtccagcgggagagagcgctgttcccctccaccaggtggtgccagtgcaggaggtgcccagggcccaggtgggggccccacgcctgatggccccatcccagtgttgctgcagatccagcccgtgcctgtgaagcaggaatcgggcacagggcctgcctcccctgggcaagccccagagaatgtcaaggttgcccagctcctggtcaacatccaggggcagaccttcgcactcgtgccccaggtggtaccctcctccaacttgaacctgccctccaagtttgtgcgcattgcccctgtgcccattgccgccaagcctgttggatcgggacccctggggcctggccctgccggtctcctcatgggccagaagttccccaagaacccagccgcagaactcatcaaaatgcacaaatgtactttccctggctgcagcaagatgtacaccaaaagcagccacctcaaggcccacctgcgccggcacacgggtgagaagcccttcgcctgcacctggccaggctgcggctggaggttctcgcgctctgacgagctgtcgcggcacaggcgctcgcactcaggtgtgaagccgtaccagtgtcctgtgtgcgagaagaagttcgcgcggagcgaccacctctccaagcacatcaaggtgcaccgcttcccgcggagcagccgctccgtgcgctccgtgaactgaaagcgccctgaaccccagcctgtccgtcaccccggatccccaccccatccccatttttttaagcaataatttatttgcctcctccagagggacatggcaatgttaccagcccaccttctgaagcctgggaggtgtgaacccagggcccgccaaccgctgcctttctcgggagtacttagagcctcgaacccgcgtccctgggggctgggccccaggcgcacggggctggaggcaggccttcgtgccttcgtgccttcgtgccttcccgcggtggccaggcctctgctgcagccgctggttgcaggcagagttttggggacctggcccttctcccactgggctcccccatcctgggccaaggccagaactttagtgctaggggaagatgaaatgtgcagttttgaaatgttgggtttccagagagagtcatgctggaggagaaggaagtaggccagaagtccagggctgcactgtggtgtgagggtggctttgtctaagatgcctgctcagcatgatcaccagagggtgtgggcaggtccctggagccgggggggggggggggggggggggggggcaggaccgggccgctgggccctcatgtgggagagaggtgaaaagcgtcccccactagggggctggcagtgcatgtgcttgagttaaatgtgcagggcagacagagccagaagggcctgtacccaggggctcgtcccctcctccggtttcccagacaaatccagacaccagcctttagggtggccttgggaggagagggccaggctgtcctgggtgtgagagaactagatagagcctcccaaccctgatttagaaatgcattccttattttgtctagaaattaataaatgaactagcttgttttgacaggtttatttcacatcctatgaatgtatgtaaataaactgtacataggtccatccacataaaatatcttttaataacatatcaacatttgtgtaaatttgaaatttaaaaaaatctatgaagctggtgtacatatgttacaattacgtatattttctttggtccttcataaaaatatatttactttgccaataaaaagaaaaagaactcaca